(19) United States
(12) Patent Application Publication
(10) Pub. No.: US 2026/0126434 A1
(43) Pub. Date: May 7, 2026

(54) METHODS AND KITS FOR MEASURING THREE OR MORE TAU EPITOPES

(71) Applicant: MESO SCALE TECHNOLOGIES, LLC., Rockville, MD (US)

(72) Inventors: John H. KENTEN, Arlington, VA (US); George SIGAL, Rockville, MD (US); Galina NIKOLENKO, Damascus, MD (US); Ryan CONNELLY, Gaithersburg, MD (US)

(21) Appl. No.: 19/238,017

(22) Filed: Jun. 13, 2025

Related U.S. Application Data
(60) Provisional application No. 63/660,171, filed on Jun. 14, 2024.
(60) Provisional application No. 63/812,415, filed on May 27, 2025.

(51) Int. Cl. G01N 33/543; G01N 33/68

(52) U.S. Cl. CPC G01N 33/543; G01N 33/6896; G01N 2440/14; G01N 2470/06; G01N 2800/2821; G01N 2800/52

(57) ABSTRACT

The disclosure relates to methods and kits for detecting, quantifying, or both, phosphorylated tau (p-tau) in a biological sample. The disclosure further provides methods for detecting brain-associated tau polypeptide, a brain injury or a neurodegenerative disease in a subject, determining the eligibility of individuals for participation in clinical trials for brain injury and/or neurodegenerative disease treatments, distinguishing between individuals with brain injury and/or neurodegenerative disease and without brain injury and/or neurodegenerative disease, and monitoring response to treatment of the same.

### FIELD OF THE INVENTION

The disclosure relates to methods and kits for detecting, quantifying, or both, phosphorylated tau (p-tau) in a biological sample. The disclosure further provides methods for detecting brain-associated tau polypeptide, a brain injury or a neurodegenerative disease in a subject, determining the eligibility of individuals for participation in clinical trials for brain injury and/or neurodegenerative disease treatments, distinguishing between individuals with and without a brain injury and/or neurodegenerative disease, and monitoring response to treatment of the same.

### REFERENCE TO ELECTRONIC SEQUENCE LISTING

The application contains a Sequence Listing which has been submitted electronically in .XML format and is hereby incorporated by reference in its entirety. Said .XML copy, created on Jun. 10, 2025, is named “0076-0090WO1.xml” and is 15,464 bytes in size. The sequence listing contained in this .XML file is part of the specification and is hereby incorporated by reference herein in its entirety.

### BACKGROUND

Neurodegenerative diseases are conditions where brain cells progressively lose their function and die, leading to a variety of symptoms and impairments. Some examples include Alzheimer's disease (AD), Parkinson's disease, and Amyotrophic Lateral Sclerosis (ALS). A traumatic brain injury (TBI) can predispose individuals to neurodegenerative diseases such as Chronic Traumatic Encephalopathy (CTE). Tau phosphorylation, a process where phosphate groups attach to the tau protein, is a key mechanism implicated in both TBI and neurodegenerative diseases. In a normal or non-disease state, tau phosphorylation helps regulate tau's interactions with microtubules. However, excessive phosphorylation (multi-phosphorylation) leads to tau detaching from microtubules and aggregating into neurofibrillary tangles (NFT). TBI can trigger this process by disrupting cellular homeostasis and interfering with tau phosphorylation, thereby increasing the risk of developing later-life neurodegenerative diseases.

AD is a progressive neurodegenerative disorder that causes dementia in approximately 10% of individuals older than 65 years. One of AD's typical brain lesions is NFTs that are thought to consist of phosphorylated forms of the microtubule associated protein tau that is assembled into paired helical filaments. Tau expression is high in non-myelinated cortical axons, especially in the regions of the brain that are involved in memory consolidation such as the limbic cortex including the hippocampus. As noted above, multi-phosphorylation of tau is thought to cause the protein to detach from the microtubules, thereby destabilizing microtubules and compromising axonal transport. While tau phosphorylation promotes axonal and synaptic plasticity in the developing brain (Lovestone et al., “The phosphorylation of tau: a critical stage in neurodevelopment and neurodegenerative processes,” Neurosciences 78(2):309-324 (1997)), it is pathological in the adult brain and specifically related to a group of disorders referred to as tauopathies, which includes AD and some forms of frontotemporal dementia (FTD) (Ballatore et al., “Tau-mediated neurodegeneration in Alzheimer's disease and related disorders,” Nature Reviews Neuroscience 8(9):663-672 (2007)).

Understanding the mechanisms of tau phosphorylation and multi-phosphorylation in TBI and neurodegenerative diseases is important for developing therapies that can target and reverse the detrimental effects of tau aggregation.

### SUMMARY OF THE INVENTION

In some aspects, the techniques described herein relate to a method of detecting, quantifying, or both, tau in a biological sample, wherein the tau comprises a plurality of detectable epitopes, the method comprising: (a) contacting the biological sample with a plurality of binding reagents, wherein the first binding reagent binds to a first epitope, the second binding reagent binds to a second epitope, and the third binding reagent binds to a third epitope; (b) forming a complex comprising the plurality of binding reagents and the tau; and (c) detecting the complex, thereby detecting the tau; or (d) measuring an amount of the complex, thereby quantifying an amount of the tau.

In some aspects, the techniques described herein relate to a method of detecting, quantifying, or both, phosphorylated tau (p-tau) in a biological sample, wherein the p-tau is phosphorylated at two or more sites, comprising: (a) contacting the biological sample with: (i) a capture reagent that a phosphorylated tau site, or a non-phosphorylated tau site; (ii) a first detection reagent that binds a phosphorylated tau site, or a non-phosphorylated tau site; and (iii) a second detection reagent that binds a phosphorylated tau site, or a non-phosphorylated tau site; (b) forming a complex comprising the capture reagent, the p-tau, the first detection reagent and the second detection reagent; and (c) detecting the complex, thereby detecting the p-tau; or (d) measuring an amount of the complex, thereby quantifying an amount of the p-tau, wherein at least two of the capture reagent, first detection reagent, and second detection reagent binds a phosphorylated tau site.

In some aspects, the techniques described herein relate to a method of detecting brain injury or brain injury severity and the brain injury is concussive injury, subconcussive injury, acute concussive injury, impact head injury, acceleration or deceleration head trauma, closed-skull neurotrauma, traumatic brain injury, stroke, seizure, status epilepticus, chronic traumatic encephalopathy (CTE), or a combination thereof.

In some aspects, the techniques described herein relate to a method of detecting a neurodegenerative disease and the neurodegenerative disease is Alzheimer's disease, amyotrophic lateral sclerosis, Friedreich ataxia, Huntington's disease, Lewy body disease, Parkinson's disease, spinal muscular atrophy, Creutzfeldt-Jakob disease, Neuronal Synuclein Disease (NSD), motor neuron disease, or any combination thereof. In some aspects, the neurodegenerative disease is Alzheimer's disease.

In some aspects, the techniques described herein relate to a method of determining eligibility of a subject to participate in a clinical trial of a therapeutic drug for preventing or delaying Alzheimer's disease, comprising: (a) obtaining a measurement of phosphorylated tau levels of the subject; and (b) determining the eligibility of the subject for the clinical trial based on the measurement of phosphorylated tau levels; wherein the subject has been diagnosed with dementia or mild cognitive impairment, or wherein the subject has subjective cognitive complaints, or wherein the subject does not have any cognitive impairment.

In some aspects, the techniques described herein relate to a method of conducting a clinical trial of a therapeutic drug or intervention for Alzheimer's disease, comprising: (a) obtaining a measurement of phosphorylated tau levels of a subject; (b) determining eligibility of the subject for the clinical trial based on the measurement of phosphorylated tau levels; and (c) administering the therapeutic drug to the subject.

In some aspects, the techniques described herein relate to a method of distinguishing a subject afflicted with Alzheimer's disease from an individual afflicted with non-Alzheimer's dementia, comprising: (a) obtaining a measurement of phosphorylated tau levels of the subject and (b) identifying, based on the measurement of phosphorylated tau levels, the subject as (i) afflicted with Alzheimer's disease or (ii) afflicted with non-Alzheimer's dementia.

In some aspects, the techniques described herein relate to a method of treating Alzheimer's disease in a subject in need thereof, comprising: (a) obtaining a measurement of phosphorylated tau levels of the subject, wherein the measurement is obtained prior to administration of a treatment for Alzheimer's disease, (b) determining, based on the measurement of phosphorylated tau levels, that the subject is afflicted with Alzheimer's disease, and (c) administering a treatment regimen for Alzheimer's disease to the subject.

In some aspects, the techniques described herein relate to a method of monitoring response to treatment for Alzheimer's disease in a subject, comprising: (a) obtaining a first measurement of phosphorylated tau levels of the subject, wherein the first measurement is obtained prior to administration of a treatment regimen for Alzheimer's disease, (b) obtaining a second measurement of phosphorylated tau levels of the subject at one or more time points after administration of the treatment regimen for Alzheimer's disease has been initiated, (c) determining, based on the first and second measurements of phosphorylated tau levels, that the subject is responding positively to the Alzheimer's treatment regimen, and (d) continuing to administer the treatment regimen for Alzheimer's disease to the subject.

In some aspects, the techniques described herein relate to a kit for detecting p-tau in a biological sample, wherein the p-tau is phosphorylated at two or more sites, comprising, in one or more vials, containers, or compartments, three reagents: (a) a capture reagent that binds a phosphorylated tau site, or a non-phosphorylated tau site; (b) a first detection reagent that binds a phosphorylated tau site, or a non-phosphorylated tau site; and (c) a second detection reagent that binds a phosphorylated tau site, or a non-phosphorylated tau site, wherein at least two of the three reagents bind a phosphorylated tau site.

In some aspects, the techniques described herein relate to a system comprising: one or more data processors; and a non-transitory computer readable storage medium containing instructions which, when executed on the one or more data processors, cause the one or more data processors to perform part or all of the methods described herein.

In some aspects, the techniques described herein relate to a computer-program product tangibly embodied in a non-transitory machine-readable storage medium, comprising instructions configured to cause one or more data processors to perform part or all of the methods described herein.

In some aspects, the techniques described herein relate to a kit for detecting, quantifying, or both, tau in a biological sample, where the tau comprises at least one detectable epitope comprising a first site, wherein the kit comprises: (a) a first binding reagent that binds to the phosphorylated state of the first site; and (b) a second binding reagent that binds to the non-phosphorylated state of the first site.

### BRIEF DESCRIPTION OF THE DRAWINGS

The following drawings form part of the present specification and are included to further demonstrate exemplary embodiments of certain aspects of the present disclosure.

FIG. 1 illustrates an exemplary illustration of a two-antibody immunoassay for detecting p-tau, comprising a capture antibody, a connector oligonucleotide (CO), and a detection antibody. In one embodiment, the capture antibody binds pTau217, and the detection antibody binds pTau231 of p-tau. FIG. 1 shows a specific exemplary embodiment in which the detection antibody comprises a nucleic acid probe (NAP) that binds the CO at the 3′ and 5′ terminal regions, thereby allowing the CO to be ligated to form a circular template oligonucleotide. In one embodiment, the nucleic acid probe is subjected to an amplification process to form an extended sequence that hybridizes to an anchoring oligonucleotide sequence on the surface. In one embodiment, the capture antibody is bound to the surface via a linker. The surface can be a bead particle or a multi-well plate. A person of ordinary skill in the art can apply the immunoassay to bind other phosphorylated and non-phosphorylated tau sites using different combinations, for e.g., as presented in Table 1.

FIG. 2 shows an exemplary illustration of a three-antibody immunoassay for detecting p-tau, comprising one capture antibody, first and second detection antibodies, and a connector oligonucleotide (CO). In one embodiment, the capture antibody binds pTau217, the first detection antibody binds pTau231, and the second detection antibody binds a third phosphorylated tau site or any amino acid of SEQ ID NO: 2 or 3. FIG. 2 shows a specific embodiment in which the capture antibody and the first and second detection antibodies comprising first and second nucleic acid probes, respectively (NAP1 and NAP2), are used to bind p-tau, thereby forming a complex on the surface. Each of the NAP1 and NAP2 binds to the CO such that the NAP1 and NAP2 are close in proximity with one another. The binding of NAP1 to the CO at the 3′ and 5′ terminal regions enables ligation of the CO to form a circular template oligonucleotide, and NAP2 is a primer for an amplification process to form an extended sequence that hybridizes to an anchoring oligonucleotide sequence on the surface. In an alternative embodiment, the capture antibody is bound to the surface via a linker. The surface can be a bead particle or a multi-well plate. A person of ordinary skill in the art can apply the immunoassay to bind other phosphorylated and non-phosphorylated tau sites using different combinations, for e.g., as presented in Table 1.

FIG. 3 shows an exemplary illustration of a three-antibody immunoassay for detecting p-tau, comprising one capture antibody, two detection antibodies, and two connector oligonucleotides (CO1 and CO2). The capture antibody binds pTau217, the first detection antibody binds pTau231, and the second detection antibody binds a third phosphorylated tau site or any amino acid of SEQ ID NO: 2 or 3. FIG. 3 shows a specific embodiment in which two detection antibodies are used to bind p-tau, each labeled with a nucleic acid probe (NAP1 and NAP2). Upon formation of the complex, NAP1 and NAP2 are in proximity to one another, and each of NAP1 and NAP2 separately binds to both CO1 and CO2 to allow ligation of CO1 and CO2 to form a circular template oligonucleotide. NAP1 and/or NAP2 are primers for an amplification process to form an extended sequence that hybridizes to an anchoring oligonucleotide sequence on the surface. One or both of NAP1 and NAP2 are capable of being extended, thereby forming first and/or second extended sequences as described herein. The surface can be a bead particle or a multi-well plate. A person of ordinary skill in the art can apply the immunoassay to bind other phosphorylated and non-phosphorylated tau sites using different combinations, for e.g., as presented in Table 1.

FIG. 4 shows an exemplary illustration of an embodiment described in FIG. 3, in which both the NAP1 and the NAP2 are capable of being extended, thereby forming first and second extended oligonucleotides as described herein. The surface can be a bead particle or a multi-well plate.

FIG. 5A shows ECL signal from the dual selective pTau181+pTau217 2-Ab immunoassay against unphosphorylated Tau441 (Total), multi-phosphorylated Tau441 (HyperPhos), multi-phosphorylated Tau441 with T181A mutation (T181A), multi-phosphorylated Tau441 with T217A mutation (T217A), or multi-phosphorylated Tau441 where all threonines are substituted with alanines except at position 217 (217T-Only).

FIG. 5B shows ECL signal from mono-selective 2-Ab immunoassays targeting pTau181, or pTau217, or a dual-selective 2-Ab immunoassay targeting both pTau181 and pTau217, using a multi-phosphorylated Tau441 calibrator.

FIG. 6 shows the data for calibration curve ECL values, Hill Slopes, LLODs and fold improvements in sensitivity for 3-Ab immunoassays detecting the following analytes: pT181, pT217, pT231, and Tau (total, Brain).

FIG. 7 shows calibration curve plots for the data in FIG. 6 for each of the analytes, illustrating the improved assay performance for the 3-Ab immunoassays (black line) over 2-Ab immunoassays (grey line).

FIG. 8A shows comparisons for the pT181, pT217, and pT181+pT217 assays performed on normal and AD-positive plasma and serum samples.

FIG. 8B shows comparisons for pT231-containing assays (pT231, pT181+pT231, pT217+pT231, and pT181+pT217+pT231) performed on normal and AD-positive plasma and serum samples.

### DETAILED DESCRIPTION OF THE INVENTION

Unless otherwise defined herein, scientific and technical terms used in the present disclosure shall have the meanings that are commonly understood by one of ordinary skill in the art.

Further, unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.” “And/or” where used herein is to be taken as specific disclosure of each of the specified features of components with or without the other. Thus, the term “and/or” as used in a phrase such as “A and/or B” herein is intended to include “A and B,” “A or B,” “A” (alone), and “B” (alone).

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” means that the specific terms recited are representative examples and embodiments of the disclosure that are not intended to be limited to the specific examples referenced or cited unless explicitly stated otherwise.

As used herein, “about” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or, “about” can mean rounded to the nearest significant digit.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

As used herein, “between” is a range inclusive of the ends of the range. For example, a number between x and y explicitly includes the numbers x and y and any numbers that fall within x and y.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)2, Fab, Fab′, and F(ab′)₂, F(ab1)₂, Fv, dAb, and Fd fragments, diabodies, and antibody-related polypeptides, so long as they exhibit the desired biological activity or function. The term “antibody or antigen-binding fragment thereof” encompasses full-length antibodies as well as truncations, variants, analogs, and derivatives thereof, e.g., naturally occurring antibodies or immunoglobulins, or engineered antibodies or fragments that bind antigens in a manner similar to antibodies. In embodiments, the antibody or antigen-binding fragment thereof includes one or more amino acid variations (e.g., point mutations) as compared to a naturally occurring counterpart, e.g., to increase stability and/or affinity. In embodiments, the binding reagent of the present disclosure includes an antibody, and the target substance is an antibody or antigen-binding fragment thereof.

In embodiments, measurement of biomarker values and levels before and after a particular event, e.g., cellular or environmental event, are used to gain information regarding an individual's response to the event. For example, samples or model organisms are subjected to stress- or disease-inducing conditions, or a treatment or prevention regimen, and a particular biomarker is then detected and quantitated in order to determine its changes in response to the condition or regimen. The opposite, i.e., measuring biomarker values and levels to determine whether an organism has been subjected to stress- or disease-inducing condition, tends to be much more complicated, as changes in the levels of a single biomarker typically cannot be definitively associated with a particular condition. However, in some cases, a single biomarker can be strongly correlated with a condition. In such cases, a highly sensitive and specific detection method for the single biomarker is important for assessing the condition and/or providing an appropriate treatment, e.g. Alzheimer's disease (AD).

As used herein, an individual with AD refers to a person who has been diagnosed with AD using a test known to one of skill in the art, for example, clinical presentation, a biomarker test, and/or a brain scan.

Reference throughout the present disclosure to “tau” or “Tau” without specifying its phosphorylation state, encompasses tau protein, or a fragment or variant thereof. Total tau (“t-tau”) encompasses phosphorylated tau (“p-tau”), non-phosphorylated tau, a fragment, or variant thereof (e.g., as recited in SEQ ID NOs: 1-9). In embodiments, total tau comprises tau epitopes and/or sites that are never phosphorylated, as defined below, and that are shared among most or all tau splice variants. In embodiments, total tau comprises tau epitopes and/or sites that are never phosphorylated, as defined below, and that are shared among most or all of SEQ ID NO: 1-9. In embodiments, p-tau encompasses tau comprising at least one phosphorylated amino acid residue or site. The term “p-tau” therefore encompasses most, if not all, isoforms of tau in humans. The term “isoform” refers to any of two or more functionally similar proteins that have similar, but not identical amino acid sequences. In embodiments, protein isoforms are produced from alternative splice variants.

As used herein, the term “fragment” of a protein (e.g., tau, p-tau, or t-tau) refers to an amino acid sequence of a protein that is shorter than the naturally-occurring sequence, N- and/or C-terminally deleted or any part of the protein deleted in comparison to the naturally occurring protein.

As used herein, the term “variant” of a protein (e.g., tau, p-tau, or t-tau) refers to a protein that shares certain structural and functional identities with another protein upon comparison by a method known in the art. For example, a variant of a protein can include a substitution, insertion, deletion, frame shift or rearrangement in another protein.

In embodiments, a variant of tau or derivative comprises a tau variant having at least about 70% identity to the full-length, mature tau protein or a fragment of tau. In embodiments, the tau or tau derivative includes one or more mutations, for example, conservative amino acid substitutions.

Naturally occurring variants include “allelic variants,” and refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level and are included in the present disclosure.

Alternative splicing, or alternative RNA splicing, or differential splicing, is an alternative process during gene expression that allows a single gene to code for multiple proteins. In this process, particular exons of a gene may be included within or excluded from the final, processed messenger RNA (mRNA) produced from that gene. This means the exons are joined in different combinations, leading to different (alternative) mRNA strands. Consequently, the proteins translated from alternatively spliced mRNAs usually contain differences in their amino acid sequence.

Alternatively, non-naturally occurring variants can be produced by mutagenesis techniques or by direct synthesis. Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Moreover, ample evidence demonstrates that variants often retain a biological activity similar to that of the naturally occurring protein.

In embodiments, variants or derivatives include, e.g., modified polypeptides. In embodiments, variants or derivatives of, e.g., polypeptides, polynucleotides, lipids, glycoproteins, are the result of chemical modification and/or endogenous modification. In embodiments, variants or derivatives are the result of in vivo modification. In embodiments, variants or derivatives are the result of in vitro modification. Modifications present in variants and derivatives include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination.

The term “epitope” or “detectable epitope” refers to a specific, localized region on the surface of tau or p-tau that is recognized by a binding reagent, e.g., capture reagent or detection reagent.

### INTRODUCTION

The methods of the disclosure provide several advantages. For instance, the methods herein have improved sensitivity, specificity, and accuracy as compared to methods described in the art. In embodiments, the disclosure provides a simple, convenient, sensitive, and specific assay with a wide dynamic range for detecting and/or quantifying tau. In embodiments, the method comprises detecting p-tau. In embodiments, the method comprises quantifying p-tau. Tau

Naturally-occurring tau is a phosphoprotein with 79-85 potential Ser and Thr phosphorylation sites on the longest tau isoform. The term “isoform” relates to any of two or more functionally similar proteins that have similar, but not identical amino acid sequences. In embodiments, the tau of the present disclosure comprises any one of the Central Nervous System (CNS) tau isoforms. In embodiments, the tau of the present disclosure comprises the 0N3R, 0N4R, 1N3R, 1N4R, 2N3R, 2N4R isoform or any combination thereof. In embodiments, the tau of the present disclosure comprises the amino acid sequence of any one of SEQ ID NOs: 1 to 9. In embodiments, the tau of the present disclosure comprises the 2N4R isoform (SEQ ID NO: 1), also referred to herein as Tau441. In embodiments, the present disclosure comprises canonical tau or “Big Tau” as defined in Fisher (“Big Tau: What We Know, and We Need to Know,” eNeuro 10(5) (2023)) and Gonzalez-Ortiz et al., (“Brain-derived tau: a novel blood-based biomarker for Alzheimer's disease-type neurodegeneration,” Brain 146:1152-1165 (2023)). Big tau is associated with the peripheral nervous system (PNS). In embodiments, the tau of the present disclosure comprises an exon 4a insert. In embodiments, the tau of the present disclosure does not comprise an exon 4a insert.

Aggregation of p-tau is indicative of neurological disorders such as Alzheimer's Disease (AD), with multi-phosphorylated, or multi-phosphorylated, tau signifying advancement of these diseases. While accumulation of multi-phosphorylated tau in cerebrospinal fluid (CSF) is an excellent early indicator of these disorders, the invasive nature of CSF collection makes serum or plasma analysis preferable despite the diminished levels of brain-derived, or brain-associated, tau compared to CSF.

In embodiments, the present disclosure provides methods for detecting and/or quantifying the amount of p-tau in a biological sample. In embodiments, the present disclosure provides methods for detecting and/or quantifying the amount of total tau (t-tau) in a biological sample. In embodiments, the term “site” refers to a single tau amino acid position or residue. In embodiments, the term “site” corresponds to an amino acid position or residue of SEQ ID NO: 1, or any isoform of tau disclosed herein or known in the art. In embodiments, the term “site” corresponds to an amino acid position or residue of any one of SEQ ID NO: 1 to 9. In embodiments, the term “site” refers to any single amino acid position of SEQ ID NO: 1 or fragment or variant thereof. In embodiments, the term “site” refers to any single amino acid position of SEQ ID NO: 2. In embodiments, the term “site” refers to any single amino acid position of SEQ ID NO: 3. In embodiments, the term “site” corresponds to amino acid position T175, T181, T205, T212, S214, T217, cis or trans T231, S293, S396, L243, or any combination thereof, of SEQ ID NO: 1.

In embodiments, the terms “tau” and “p-tau” are used interchangeably unless the context indicates otherwise to one of ordinary skill in the art. In embodiments, the terms “tau” or “p-tau” refers to an amino acid sequence at least 80%, 85%, 86%, 87%, 88%, 89%, 90%, 91%, 92%, 93%, 94%, 95%, 96%, 97%, 98%, 99% or 100% identity to the amino acid sequence of SEQ ID NO: 1. In embodiments, the terms “tau” or “p-tau” comprises an amino acid sequence at least 80%, 85%, 86%, 87%, 88%, 89%, 90%, 91%, 92%, 93%, 94%, 95%, 96%, 97%, 98%, 99% or 100% identity to the amino acid sequence of SEQ ID NO: 2. In embodiments, the terms “tau” or “p-tau” comprises an amino acid sequence at least 80%, 85%, 86%, 87%, 88%, 89%, 90%, 91%, 92%, 93%, 94%, 95%, 96%, 97%, 98%, 99% or 100% identity to the amino acid sequence of SEQ ID NO: 3. In embodiments, the terms “tau” or “p-tau” refers to an amino acid sequence at least 80%, 85%, 86%, 87%, 88%, 89%, 90%, 91%, 92%, 93%, 94%, 95%, 96%, 97%, 98%, 99% or 100% identity to the amino acid sequence of SEQ ID NO: 4. In embodiments, the terms “tau” or “p-tau” refers to an amino acid sequence at least 80%, 85%, 86%, 87%, 88%, 89%, 90%, 91%, 92%, 93%, 94%, 95%, 96%, 97%, 98%, 99% or 100% identity to the amino acid sequence of SEQ ID NO: 5. In embodiments, the terms “tau” or “p-tau” refers to an amino acid sequence at least 80%, 85%, 86%, 87%, 88%, 89%, 90%, 91%, 92%, 93%, 94%, 95%, 96%, 97%, 98%, 99% or 100% identity to the amino acid sequence of SEQ ID NO: 6. In embodiments, the terms “tau” or “p-tau” refers to an amino acid sequence at least 80%, 85%, 86%, 87%, 88%, 89%, 90%, 91%, 92%, 93%, 94%, 95%, 96%, 97%, 98%, 99% or 100% identity to the amino acid sequence of SEQ ID NO: 7. In embodiments, the terms “tau” or “p-tau” refers to an amino acid sequence at least 80%, 85%, 86%, 87%, 88%, 89%, 90%, 91%, 92%, 93%, 94%, 95%, 96%, 97%, 98%, 99% or 100% identity to the amino acid sequence of SEQ ID NO: 8. In embodiments, the terms “tau” or “p-tau” refers to an amino acid sequence at least 80%, 85%, 86%, 87%, 88%, 89%, 90%, 91%, 92%, 93%, 94%, 95%, 96%, 97%, 98%, 99% or 100% identity to the amino acid sequence of SEQ ID NO: 9.

For any given isoform of tau or p-tau, the expression “capable of being phosphorylated” as used herein refers to a tau or p-tau site that comprises any one of the serine (S), threonine (T), and tyrosine (Y) tau sites capable of being phosphorylated, i.e., can be present in either a phosphorylated state or a non-phosphorylated state. In embodiments, up to eighty-five of any one of the serine (S), threonine (T), and tyrosine (Y) tau sites are capable of being phosphorylated. For any given isoform of tau or p-tau, the expression “commonly phosphorylated” refers to sites that are phosphorylated in a non-disease state. For any given isoform of tau or p-tau, the expression “rarely phosphorylated” refers to sites that are phosphorylated in a disease state. For any given isoform of tau or p-tau, the expression “never phosphorylated” refers to sites that are not phosphorylated in any disease or non-disease state. In embodiments, the “never phosphorylated” site comprises any one of the other tau sites that are not one of the eighty-five serine (S), threonine (T), and tyrosine (Y) tau sites capable of being phosphorylated, as discussed herein.

“Identity” as known in the art is the relationship between two or more polypeptide sequences or two or more polynucleotide sequences, as determined by comparing the sequences. In the art, identity also means the degree of sequence relatedness between polypeptide or polynucleotide sequences, as the case can be, as determined by the match between strings of such sequences. While there exist a number of methods to measure identity between two polypeptide or two polynucleotide sequences, methods commonly employed to determine identity are codified in computer programs. Preferred computer programs to determine identity between two sequences include, but are not limited to, GCG program package (Devereux, et al., Nucleic Acids Research, 12, 387 (1984), BLASTP, BLASTN/NBLAST, FASTA (Atschul et al., J. Molec. Biol. 215, 403 (1990)), CLUSTAL, XBLAST/BLASTx, PSI-Blast, ALIGN, ADVANCE, and ADAM.

Mutations, including conservation and tolerated substitutions, insertions, and deletions, can be introduced into the sequences provided using any appropriate method including, but not limited to, those based on polymerase chain reaction (PCR), restriction enzyme-based cloning, or ligation independent cloning (LIC) procedures. These methods are detailed in many of the standard molecular biology texts.

In embodiments, any one of the amino acid residues or sites of tau or p-tau referenced herein correspond to SEQ ID NO: 1, or a fragment or variant thereof. In embodiments, any one of the amino acid residues or sites of tau or p-tau referenced herein correspond to SEQ ID NO: 2, or a fragment or variant thereof. In embodiments, any one of the amino acid residues or sites of tau or p-tau referenced herein correspond to SEQ ID NO: 3, or a fragment or variant thereof. In embodiments, any one of the amino acid residues or sites of tau or p-tau referenced herein correspond to SEQ ID NO: 4, or a fragment or variant thereof. In embodiments, any one of the amino acid residues or sites of tau or p-tau referenced herein correspond to SEQ ID NO: 5, or a fragment or variant thereof. In embodiments, any one of the amino acid residues or sites of tau or p-tau referenced herein correspond to SEQ ID NO: 6, or a fragment or variant thereof. In embodiments, any one of the amino acid residues or sites of tau or p-tau referenced herein correspond to SEQ ID NO: 7, or a fragment or variant thereof. In embodiments, any one of the amino acid residues or sites of tau or p-tau referenced herein correspond to SEQ ID NO: 8, or a fragment or variant thereof. In embodiments, any one of the amino acid residues or sites of tau or p-tau referenced herein correspond to SEQ ID NO: 9, or a fragment or variant thereof. In embodiments, the tau or p-tau comprises the following amino acid positions or sites: T175, T181, T205, T212, S214, T217, cis or trans T231, S293, S396, or any combination thereof of SEQ ID NO: 1. In embodiments, the tau or p-tau comprises L243 of SEQ ID NO: 1. In embodiments, the tau or p-tau comprises a brain-associated polypeptide sequence. In embodiments, the tau or p-tau comprises SEQ ID NO: 2. In embodiments, the tau comprises SEQ ID NO: 3.

In embodiments, the tau or p-tau is phosphorylated at about three to about eighty-five sites. In embodiments, the p-tau is phosphorylated at about ten to about sixty sites. In embodiments, the p-tau is phosphorylated at about twenty to about forty sites. In embodiments, the p-tau is phosphorylated at about three sites. In embodiments, the p-tau is phosphorylated at about ten sites. In embodiments, the p-tau is phosphorylated at about twenty-nine sites. In embodiments, the p-tau is phosphorylated at about thirty-eight sites. In embodiments, the p-tau is phosphorylated at about forty sites. In embodiments, the p-tau is phosphorylated at about fifty-five sites. In embodiments, the p-tau is phosphorylated at about seventy-nine sites. In embodiments, the p-tau is phosphorylated at about eighty-five sites. In embodiments, the term “multi-phosphorylated tau” refers to tau or p-tau that is present in a disease state.

In embodiments, the p-tau is phosphorylated at amino acid position T175 (pTau175), T181 (pTau181), T205 (pTau205), T212 (pTau212), S214 (pTau214), T217 (pTau217), cis or trans T231 (pTau231), S293 (pTau293), S396 (pTau396), or any combination thereof, wherein the amino acid position corresponds to SEQ ID NO: 1. In embodiments, the p-tau comprises L243 (Tau243). In embodiments, p-tau comprises pTau217, pTau181, pTau231, pTau205, pTau212, or any combination thereof. In embodiments, the p-tau is phosphorylated at any serine or threonine residue of SEQ ID NO: 2. In embodiments, the p-tau is phosphorylated at any serine or threonine residue of SEQ ID NO: 3. In embodiments, the present disclosure further provides methods for detecting and/or quantifying the amount of total tau in a sample, wherein the total tau comprises non-phosphorylated tau and p-tau, and wherein the p-tau is phosphorylated at any of its serine (Ser) or threonine (Thr) residues. In embodiments, the p-tau is phosphorylated at amino acid position T231 and T217 (referred to herein as “pTau231” and “pTau217”).

In embodiments, the disclosure provides a method of detecting and/or quantifying the amount of p-tau in a biological sample, comprising: (a) contacting the biological sample with: (i) a capture reagent that binds a phosphorylated tau site or a non-phosphorylated tau site, wherein the capture reagent is on a surface, and the surface further comprises an anchoring reagent; (ii) a first detection reagent that binds a phosphorylated tau site or a non-phosphorylated tau site, wherein the first detection reagent comprises a first nucleic acid probe; and (iii) a second detection reagent that binds a phosphorylated tau site or a non-phosphorylated tau site, wherein the second detection reagent comprises a second nucleic acid probe; (b) forming a complex on the surface comprising the capture reagent, p-tau, and the first and second detection reagents; (c) using an extension process that requires the first and second nucleic acid probes to be in proximity, extending the first and/or second nucleic acid probe to form an extended sequence comprising an anchoring region that binds to the anchoring reagent; (d) binding the extended sequence to the anchoring reagent; and (e) detecting and/or measuring the amount of the extended sequence bound to the surface, thereby detecting and/or quantifying the amount of p-tau.

In embodiments, the disclosure provides a method of detecting and/or quantifying the amount of p-tau in a biological sample, comprising: (a) contacting the biological sample with: (i) a capture reagent that binds a phosphorylated tau site or a non-phosphorylated tau site, wherein the capture reagent is on a surface, and the surface further comprises an anchoring reagent; and (ii) a detection reagent that binds a phosphorylated tau site or a non-phosphorylated tau site, wherein the detection reagent comprises a nucleic acid probe; (b) forming a complex on the surface comprising the capture reagent, p-tau, and the detection reagent; (c) extending the nucleic acid probe to form an extended sequence comprising an anchoring region that binds to the anchoring reagent; (d) binding the extended sequence to the anchoring reagent; and (e) detecting and/or measuring the amount of extended sequence bound to the surface, thereby detecting and/or quantifying the amount of p-tau.

In embodiments, the disclosure provides a method of detecting and/or quantifying the amount of p-tau in a biological sample, comprising: (a) contacting the biological sample with: (i) a capture reagent that binds a phosphorylated tau site or a non-phosphorylated tau site, wherein the capture reagent is on a surface; and (ii) a detection reagent that binds a phosphorylated tau site or a non-phosphorylated tau site, wherein the detection reagent comprises a detectable label; (b) forming a complex on the surface comprising the capture reagent, p-tau, and the detection reagent; and (c) detecting and/or measuring the amount of detectable label on the surface, thereby detecting and/or quantifying the amount of p-tau.

In embodiments, the disclosure provides a method of detecting and/or quantifying the amount of p-tau in a biological sample, comprising: (a) contacting the biological sample with: (i) a capture reagent that binds a phosphorylated tau site or a non-phosphorylated tau site, wherein the capture reagent is on a surface; (ii) a first detection reagent that binds a phosphorylated tau site or a non-phosphorylated tau site; and (iii) a second detection reagent that binds a phosphorylated tau site or a non-phosphorylated tau site, wherein the first and/or second detection reagent comprises a detectable label; (b) forming a complex on the surface comprising the capture reagent, p-tau, the first detection reagent and the second detection reagent; and (c) detecting and/or measuring the amount of detectable label on the surface, thereby detecting and/or quantifying the amount of p-tau.

In embodiments, the disclosure provides a method of detecting and/or quantifying p-tau in a biological sample, wherein the p-tau is phosphorylated at two or more sites, comprising: (a) contacting the biological sample with: (i) a capture reagent that binds a phosphorylated tau site, or a non-phosphorylated tau site; (ii) a first detection reagent that binds a phosphorylated tau site, or a non-phosphorylated tau site; and (iii) a second detection reagent that binds a phosphorylated tau site, or a non-phosphorylated tau site; (b) forming a complex comprising the capture reagent, the p-tau, the first detection reagent and the second detection reagent; and (c) detecting the complex, thereby detecting the p-tau; or (d) measuring an amount of the complex, thereby quantifying an amount of the p-tau, wherein at least two of the capture reagent, first detection reagent, and second detection reagent binds a phosphorylated tau site.

In embodiments, the capture reagent binds pTau217, the first detection reagent binds pTau231, and the second detection reagent binds a third phosphorylated tau site or a non-phosphorylated tau site of SEQ ID NO: 1, or any site of the brain-associated tau polypeptide of SEQ ID NO: 2 or 3.

In embodiments, the capture reagent binds pTau231, the first detection reagent binds pTau217, and the second detection reagent binds a third phosphorylated tau site or a non-phosphorylated tau site of SEQ ID NO: 1, or any site of the brain-associated tau polypeptide of SEQ ID NO: 2 or 3.

In embodiments, the capture reagent binds pTau217, the first detection reagent binds pTau231, and the second detection reagent binds pTau181 or pTau243 with reference to SEQ ID NO: 1, or any site of the brain-associated tau polypeptide corresponding to SEQ ID NO: 2 or 3.

In embodiments, the present disclosure provides a method of detecting, quantifying, or both, tau in a biological sample, wherein the tau comprises a plurality of detectable epitopes (also referred to as epitopes), the method comprising: (a) contacting the biological sample with a plurality of binding reagents comprising at least three binding reagents, wherein the first binding reagent binds to a first epitope, the second binding reagent binds to a second epitope, and the third binding reagent binds to a third epitope; (b) forming a complex comprising the plurality of binding reagents and the tau; and (c) detecting the complex, thereby detecting the tau; or (d) measuring an amount of the complex, thereby quantifying an amount of the tau. In embodiments, the first epitope comprises a first site capable of being phosphorylated. In embodiments, the second epitope comprises a second site capable of being phosphorylated. In embodiments, the third epitope comprises a third site capable of being phosphorylated. In embodiments, the first epitope comprises a first site commonly phosphorylated. In embodiments, the second epitope comprises a second site commonly phosphorylated. In embodiments, the third epitope comprises a third site commonly phosphorylated. In embodiments, the first epitope comprises a first site rarely or never phosphorylated. In embodiments, the second epitope comprises a second site rarely or never phosphorylated. In embodiments, the third epitope comprises a third site rarely or never phosphorylated.

In embodiments, the first epitope comprises a first site capable of being phosphorylated. In embodiments, the first site comprises any one of the eighty-five serine (S), threonine (T), and tyrosine (Y) tau sites capable of being phosphorylated. In embodiments, the first site is selected from: T175, T181, T205, T212, 5214, T217, cis or trans T231, S293, or S396, of SEQ ID NO: 1. In embodiments, the first binding reagent binds to the phosphorylated state and/or binds to the non-phosphorylated state of the first site. In embodiments, the first binding reagent binds to a commonly phosphorylated site. In embodiments, the first binding reagent binds to a rarely phosphorylated site. In embodiments, the first binding reagent binds to a never phosphorylated site. In embodiments, the first site comprises any one of the other tau sites that are not one of the eighty-five serine (S), threonine (T), and tyrosine (Y) tau sites capable of being phosphorylated.

In embodiments, the second epitope comprises a second site capable of being phosphorylated. In embodiments, the second site comprises any one of the eighty-five serine (S), threonine (T), and tyrosine (Y) tau sites capable of being phosphorylated. In embodiments, the second site is selected from: T175, T181, T205, T212, S214, T217, cis or trans T231, S293, or S396, of SEQ ID NO: 1. In embodiments, the second binding reagent binds to the phosphorylated state and/or binds to the non-phosphorylated state of the second site capable of being phosphorylated. In embodiments, the second binding reagent binds to a commonly phosphorylated site. In embodiments, the second binding reagent binds to a rarely phosphorylated site. In embodiments, the second binding reagent binds to a never phosphorylated site. In embodiments, the second site comprises any one of the other tau sites that are not one of the eighty-five serine (S), threonine (T), and tyrosine (Y) tau sites capable of being phosphorylated.

In embodiments, the third epitope comprises a third site capable of being phosphorylated. In embodiments, the third site comprises any one of the eighty-five serine (S), threonine (T), and tyrosine (Y) tau sites capable of being phosphorylated. In embodiments, the third site is selected from: T175, T181, T205, T212, S214, T217, cis or trans T231, S293, or S396 of SEQ ID NO: 1. In embodiments, the third binding reagent binds to the phosphorylated state and/or binds to the non-phosphorylated state of the third site capable of being phosphorylated. In embodiments, the first binding reagent binds to a commonly phosphorylated site. In embodiments, the first binding reagent binds to a rarely phosphorylated site. In embodiments, the first binding reagent binds to a never phosphorylated site. In embodiments, the third site comprises any one of the other tau sites that are not one of the eighty-five serine (S), threonine (T), and tyrosine (Y) tau sites capable of being phosphorylated.

In embodiments, the level of p-tau is measured by performing an electrochemiluminescence (ECL) assay on a plate reading system, e.g., a MESO SCALE DISCOVERY® plate reading system such as the MESO SECTOR S 600® or MESO QUICKPLEX SQ 120@. In embodiments, the level of p-tau is measured using a MESO SCALE DISCOVERY® ECL assay platform. In embodiments, the level of p-tau is measured using MESO SCALE DISCOVERY® Kit Catalog No. K151AGMS.

In embodiments, the method utilizes one capture reagent and one or more detection reagents that bind at least two phosphorylated tau sites of p-tau in a particular configuration as described herein. In embodiments, the reagents bind p-tau with higher affinity and greater specificity as compared with binding a single phosphorylation site, thereby providing improved sensitivity and specificity. In embodiments, the capture reagent is capable of binding p-tau or non-phosphorylated tau. In embodiments, at least one detection reagent binds p-tau but not non-phosphorylated tau. In embodiments, the method has decreased cross-reactivity with non-phosphorylated tau and improved accuracy for p-tau.

In embodiments, the disclosure provides a simple, convenient, sensitive, and specific assay with a wide dynamic range for detecting and/or quantifying t-tau. In embodiments, the method utilizes a capture reagent and one or more detection reagents that bind at least two phosphorylated tau sites. In embodiments, the method utilizes one or more capture reagents and/or one or more detection reagents that binds to a non-phosphorylated tau site. In embodiments, the reagents bind tau with higher affinity and greater specificity as compared with binding a single phosphorylation site, thereby improving sensitivity and specificity. In embodiments, the capture reagent is capable of binding p-tau or non-phosphorylated tau.

The methods disclosed herein are able to detect different tau sites or epitopes. For example, some tau sites are rarely or never phosphorylated, whereas some tau sites are capable of being phosphorylated and are therefore either in a phosphorylated or a non-phosphorylated state. For sites that are capable of being phosphorylated, the method utilizes specific capture and/or detection reagents that bind to each specific state, i.e., phosphorylated or non-phosphorylated, with minimal or no cross-reactivity. In embodiments, the capture reagent and/or the one or more detection reagents that binds to a phosphorylated site of tau does not bind to the non-phosphorylated state of the same site. In embodiments, the capture reagent and/or the one or more detection reagents that binds to a non-phosphorylated state of a tau site may also bind to the phosphorylated state of the tau site. In embodiments, the capture reagent and/or the one or more detection reagents that binds to a non-phosphorylated state of a tau site does not bind to the phosphorylated state of the tau site. In embodiments, the one or more detection reagents comprises one detection reagent. In embodiments, the one or more detection reagents comprises a first detection reagent and a second detection reagent, and optionally a third or more detection reagents as described herein.

In embodiments, at least one detection reagent binds to a phosphorylated tau site and also binds to the non-phosphorylated tau site. In embodiments, at least one detection reagent binds to phosphorylated tau site but not to the non-phosphorylated tau site. In embodiments, the method has decreased cross-reactivity with non-phosphorylated tau and improved accuracy for p-tau. The present disclosure provides an improved method for detecting or analyzing a single tau molecule and its phosphorylation state as compared to conventional methods in the art, e.g., mass spectrometry (MS). MS for analyzing tau phosphorylation faces challenges including the difficulty of distinguishing between similar phosphorylation sites, the need for high-resolution separation techniques, and the complexity of interpreting results in complex mixtures like brain tissue. An important limitation of MS is that it requires splitting single proteins into peptides, or a whole population of proteins into peptides and therefore does not provide a method of determining the phosphorylation state of a single protein or population of proteins. In embodiments, the methods of the present invention are capable of determining the phosphorylation state of a single tau protein and/or a population of identically phosphorylated tau proteins, e.g., by selecting specific combinations of capture and detection reagents capable of binding to specific tau sites as described herein.

In embodiments, the disclosure provides a three-antibody immunoassay for detecting and/or quantifying p-tau, comprising a capture reagent, a first detection reagent and a second detection reagent, wherein at least two of the capture reagent, the first detection reagent, and the second detection reagent bind to phosphorylated tau sites, and the remaining reagent binds to a non-phosphorylated tau site. In embodiments, each of the capture reagent, the first detection reagent, and the second detection reagent binds to a phosphorylated tau site. In embodiments, the three-antibody immunoassay binds to p-tau according to any one of the combinations shown in Table 1. In embodiments, at least one of the capture reagent, the first detection reagent, and the second detection reagent binds to an amino acid position or site of any one of SEQ ID NO: 1-9. In embodiments, at least one of the capture reagent, the first detection reagent, and the second detection reagent binds to an amino acid position or site of SEQ ID NO: 1. In embodiments, at least one of the capture reagent, the first detection reagent, and the second detection reagent binds an amino acid position or site of SEQ ID NO: 2. In embodiments, at least one of the capture reagent, the first detection reagent, and the second detection reagent binds an amino acid position or site of SEQ ID NO: 3. In embodiments, at least one of the capture reagent, the first detection reagent, and the second detection reagent binds to an amino acid position or site of SEQ ID NO: 4. In embodiments, at least one of the capture reagent, the first detection reagent, and the second detection reagent binds to an amino acid position or site of SEQ ID NO:5. In embodiments, at least one of the capture reagent, the first detection reagent, and the second detection reagent binds to an amino acid position or site of SEQ ID NO: 6. In embodiments, at least one of the capture reagent, the first detection reagent, and the second detection reagent binds to an amino acid position or site of SEQ ID NO: 7. In embodiments, at least one of the capture reagent, the first detection reagent, and the second detection reagent binds to an amino acid position or site of SEQ ID NO: 8. In embodiments, at least one of the capture reagent, the first detection reagent, and the second detection reagent binds to an amino acid position or site of SEQ ID NO: 9.

In embodiments, the disclosure provides a three-antibody immunoassay for detecting and/or quantifying tau comprising: a first binding reagent, a second binding reagent, and a third binding reagent. In embodiments, the three binding reagents bind to tau at any one of the epitopes or sites of any one of SEQ ID NOs: 1-9. In embodiments, the three binding reagents bind to one or more sites capable of being phosphorylated. In embodiments, the three binding reagents bind to one or more sites that are rarely or never phosphorylated. In embodiments, the three binding reagents bind to sites that are commonly phosphorylated.

In embodiments, the three binding reagents bind to tau according to any one of the combinations shown in Table 2. Table 2 refers to the possible combinations of three binding reagents that can bind to the following tau residues or sites: T175, T181, T205, T212, S214, T217, T231, S293, S396, and a brain-associated epitope (“BE-Tau”).

In embodiments, the tau sites in Table 2 may be in a phosphorylated state, a non-phosphorylated state, or any combination thereof. In embodiments of Table 2, the site bound by the first, second, and third binding reagents is non-phosphorylated. In embodiments of Table 2, the site bound by the first and second binding reagents is non-phosphorylated, and the site bound by the third binding reagent is phosphorylated. In embodiments of Table 2, the site bound by the first and third binding reagents is non-phosphorylated, and the site bound by the second binding reagent is phosphorylated. In embodiments of Table 2, the site bound by the second and third binding reagents is non-phosphorylated, and the site bound by the first binding reagent is phosphorylated. In embodiments of Table 2, the site bound by the first and second binding reagents is phosphorylated, and the site bound by the third binding reagent is non-phosphorylated. In embodiments of Table 2, the site bound by the first and third binding reagents is phosphorylated, and the site bound by the second binding reagent is non-phosphorylated. In embodiments of Table 2, the site bound by the second and third binding reagents is phosphorylated, and the site bound by the first binding reagent is non-phosphorylated. In embodiments of Table 2, the site bound by the first, second, and third binding reagents is phosphorylated. In embodiments, the three binding reagents may bind according to any of the tau site combinations in Table 2 as follows:

| 1^{st} Binding Reagent | 2^{nd} Binding Reagent | 3^{rd} Binding Reagent |
|---|---|---|
| Non-phosphorylated site | Non-phosphorylated site | Non-phosphorylated site |
| Non-phosphorylated site | Non-phosphorylated site | Phosphorylated site |
| Non-phosphorylated site | Phosphorylated site | Non-phosphorylated site |
| Phosphorylated site | Non-phosphorylated site | Non-phosphorylated site |
| Phosphorylated site | Phosphorylated site | Non-phosphorylated site |
| Phosphorylated site | Non-phosphorylated site | Phosphorylated site |
| Non-phosphorylated site | Phosphorylated site | Phosphorylated site |
| Phosphorylated site | Phosphorylated site | Phosphorylated site |

In embodiments, one or more of the three binding reagents comprises a capture reagent. In embodiments, one or more of the three binding reagents comprises a capture reagent immobilized to a surface. In embodiments, one or more of the three binding reagents comprises a detection reagent. In embodiments, one or more of the three binding reagents is conjugated to a nucleic acid probe as described herein.

TABLE 1

| Capture | Detection | Capture | Detection | Capture | Detection |
|---|---|---|---|---|---|
| Reagent | Reagents Pair | Reagent | Reagents Pair | Reagent | Reagents Pair |
| pTau175 | pTau181/pTau205 | pTau181 | pTau175/pTau205 | pTau205 | pTau175/pTau181 |
| pTau175 | pTau181/pTau212 | pTau181 | pTau175/pTau212 | pTau205 | pTau175/pTau212 |
| pTau175 | pTau181/pTau214 | pTau181 | pTau175/pTau214 | pTau205 | pTau175/pTau214 |
| pTau175 | pTau181/pTau217 | pTau181 | pTau175/pTau217 | pTau205 | pTau175/pTau217 |
| pTau175 | pTau181/pTau231 | pTau181 | pTau175/pTau231 | pTau205 | pTau175/pTau231 |
| pTau175 | pTau181/pTau293 | pTau181 | pTau175/pTau293 | pTau205 | pTau175/pTau293 |
| pTau175 | pTau181/pTau396 | pTau181 | pTau175/pTau396 | pTau205 | pTau175/pTau396 |
| pTau175 | pTau205/pTau212 | pTau181 | pTau205/pTau212 | pTau205 | pTau181/pTau212 |
| pTau175 | pTau205/pTau214 | pTau181 | pTau205/pTau214 | pTau205 | pTau181/pTau214 |
| pTau175 | pTau205/pTau217 | pTau181 | pTau205/pTau217 | pTau205 | pTau181/pTau217 |
| pTau175 | pTau205/pTau231 | pTau181 | pTau205/pTau231 | pTau205 | pTau181/pTau231 |
| pTau175 | pTau205/pTau293 | pTau181 | pTau205/pTau293 | pTau205 | pTau181/pTau293 |
| pTau175 | pTau205/pTau396 | pTau181 | pTau205/pTau396 | pTau205 | pTau181/pTau396 |
| pTau175 | pTau212/pTau214 | pTau181 | pTau212/pTau214 | pTau205 | pTau212/pTau214 |
| pTau175 | pTau212/pTau217 | pTau181 | pTau212/pTau217 | pTau205 | pTau212/pTau217 |
| pTau175 | pTau212/pTau231 | pTau181 | pTau212/pTau231 | pTau205 | pTau212/pTau231 |
| pTau175 | pTau212/pTau293 | pTau181 | pTau212/pTau293 | pTau205 | pTau212/pTau293 |
| pTau175 | pTau212/pTau396 | pTau181 | pTau212/pTau396 | pTau205 | pTau212/pTau396 |
| pTau175 | pTau214/pTau217 | pTau181 | pTau214/pTau217 | pTau205 | pTau214/pTau217 |
| pTau175 | pTau214/pTau231 | pTau181 | pTau214/pTau231 | pTau205 | pTau214/pTau231 |
| pTau175 | pTau214/pTau293 | pTau181 | pTau214/pTau293 | pTau205 | pTau214/pTau293 |
| pTau175 | pTau214/pTau396 | pTau181 | pTau214/pTau396 | pTau205 | pTau214/pTau396 |
| pTau175 | pTau217/pTau231 | pTau181 | pTau217/pTau231 | pTau205 | pTau217/pTau231 |
| pTau175 | pTau217/pTau293 | pTau181 | pTau217/pTau293 | pTau205 | pTau217/pTau293 |
| pTau175 | pTau217/pTau396 | pTau181 | pTau217/pTau396 | pTau205 | pTau217/pTau396 |
| pTau175 | pTau231/pTau293 | pTau181 | pTau231/pTau293 | pTau205 | pTau231/pTau293 |
| pTau175 | pTau231/pTau396 | pTau181 | pTau231/pTau396 | pTau205 | pTau231/pTau396 |
| pTau175 | pTau293/pTau396 | pTau181 | pTau293/pTau396 | pTau205 | pTau293/pTau396 |
| pTau212 | pTau175/pTau181 | pTau214 | pTau175/pTau181 | pTau217 | pTau175/pTau181 |
| pTau212 | pTau175/pTau205 | pTau214 | pTau175/pTau205 | pTau217 | pTau175/pTau205 |
| pTau212 | pTau175/pTau214 | pTau214 | pTau175/pTau212 | pTau217 | pTau175/pTau212 |
| pTau212 | pTau175/pTau217 | pTau214 | pTau175/pTau217 | pTau217 | pTau175/pTau214 |
| pTau212 | pTau175/pTau231 | pTau214 | pTau175/pTau231 | pTau217 | pTau175/pTau231 |
| pTau212 | pTau175/pTau293 | pTau214 | pTau175/pTau293 | pTau217 | pTau175/pTau293 |
| pTau212 | pTau175/pTau396 | pTau214 | pTau175/pTau396 | pTau217 | pTau175/pTau396 |
| pTau212 | pTau181/pTau205 | pTau214 | pTau181/pTau205 | pTau217 | pTau181/pTau205 |
| pTau212 | pTau181/pTau214 | pTau214 | pTau181/pTau212 | pTau217 | pTau181/pTau212 |
| pTau212 | pTau181/pTau217 | pTau214 | pTau181/pTau217 | pTau217 | pTau181/pTau214 |
| pTau212 | pTau181/pTau231 | pTau214 | pTau181/pTau231 | pTau217 | pTau181/pTau231 |
| pTau212 | pTau181/pTau293 | pTau214 | pTau181/pTau293 | pTau217 | pTau181/pTau293 |
| pTau212 | pTau181/pTau396 | pTau214 | pTau181/pTau396 | pTau217 | pTau181/pTau396 |
| pTau212 | pTau205/pTau214 | pTau214 | pTau205/pTau212 | pTau217 | pTau205/pTau212 |
| pTau212 | pTau205/pTau217 | pTau214 | pTau205/pTau217 | pTau217 | pTau205/pTau214 |
| pTau212 | pTau205/pTau231 | pTau214 | pTau205/pTau231 | pTau217 | pTau205/pTau231 |
| pTau212 | pTau205/pTau293 | pTau214 | pTau205/pTau293 | pTau217 | pTau205/pTau293 |
| pTau212 | pTau205/pTau396 | pTau214 | pTau205/pTau396 | pTau217 | pTau205/pTau396 |
| pTau212 | pTau214/pTau217 | pTau214 | pTau212/pTau217 | pTau217 | pTau212/pTau214 |
| pTau212 | pTau214/pTau231 | pTau214 | pTau212/pTau231 | pTau217 | pTau212/pTau231 |
| pTau212 | pTau214/pTau293 | pTau214 | pTau212/pTau293 | pTau217 | pTau212/pTau293 |
| pTau212 | pTau214/pTau396 | pTau214 | pTau212/pTau396 | pTau217 | pTau212/pTau396 |
| pTau212 | pTau217/pTau231 | pTau214 | pTau217/pTau231 | pTau217 | pTau214/pTau231 |
| pTau212 | pTau217/pTau293 | pTau214 | pTau217/pTau293 | pTau217 | pTau214/pTau293 |
| pTau212 | pTau217/pTau396 | pTau214 | pTau217/pTau396 | pTau217 | pTau214/pTau396 |
| pTau212 | pTau231/pTau293 | pTau214 | pTau231/pTau293 | pTau217 | pTau231/pTau293 |
| pTau212 | pTau231/pTau396 | pTau214 | pTau231/pTau396 | pTau217 | pTau231/pTau396 |
| pTau212 | pTau293/pTau396 | pTau214 | pTau293/pTau396 | pTau217 | pTau293/pTau396 |
| pTau231 | pTau175/pTau181 | pTau293 | pTau175/pTau181 | pTau396 | pTau175/pTau181 |
| pTau231 | pTau175/pTau205 | pTau293 | pTau175/pTau205 | pTau396 | pTau175/pTau205 |
| pTau231 | pTau175/pTau212 | pTau293 | pTau175/pTau212 | pTau396 | pTau175/pTau212 |
| pTau231 | pTau175/pTau214 | pTau293 | pTau175/pTau214 | pTau396 | pTau175/pTau214 |
| pTau231 | pTau175/pTau217 | pTau293 | pTau175/pTau217 | pTau396 | pTau175/pTau217 |
| pTau231 | pTau175/pTau293 | pTau293 | pTau175/pTau231 | pTau396 | pTau175/pTau231 |
| pTau231 | pTau175/pTau396 | pTau293 | pTau175/pTau396 | pTau396 | pTau175/pTau293 |
| pTau231 | pTau181/pTau205 | pTau293 | pTau181/pTau205 | pTau396 | pTau181/pTau205 |
| pTau231 | pTau181/pTau212 | pTau293 | pTau181/pTau212 | pTau396 | pTau181/pTau212 |
| pTau231 | pTau181/pTau214 | pTau293 | pTau181/pTau214 | pTau396 | pTau181/pTau214 |
| pTau231 | pTau181/pTau217 | pTau293 | pTau181/pTau217 | pTau396 | pTau181/pTau217 |
| pTau231 | pTau181/pTau293 | pTau293 | pTau181/pTau231 | pTau396 | pTau181/pTau231 |
| pTau231 | pTau181/pTau396 | pTau293 | pTau181/pTau396 | pTau396 | pTau181/pTau293 |
| pTau231 | pTau205/pTau212 | pTau293 | pTau205/pTau212 | pTau396 | pTau205/pTau212 |
| pTau231 | pTau205/pTau214 | pTau293 | pTau205/pTau214 | pTau396 | pTau205/pTau214 |
| pTau231 | pTau205/pTau217 | pTau293 | pTau205/pTau217 | pTau396 | pTau205/pTau217 |
| pTau231 | pTau205/pTau293 | pTau293 | pTau205/pTau231 | pTau396 | pTau205/pTau231 |
| pTau231 | pTau205/pTau396 | pTau293 | pTau205/pTau396 | pTau396 | pTau205/pTau293 |
| pTau231 | pTau212/pTau214 | pTau293 | pTau212/pTau214 | pTau396 | pTau212/pTau214 |
| pTau231 | pTau212/pTau217 | pTau293 | pTau212/pTau217 | pTau396 | pTau212/pTau217 |
| pTau231 | pTau212/pTau293 | pTau293 | pTau212/pTau231 | pTau396 | pTau212/pTau231 |
| pTau231 | pTau212/pTau396 | pTau293 | pTau212/pTau396 | pTau396 | pTau212/pTau293 |
| pTau231 | pTau214/pTau217 | pTau293 | pTau214/pTau217 | pTau396 | pTau214/pTau217 |
| pTau231 | pTau214/pTau293 | pTau293 | pTau214/pTau231 | pTau396 | pTau214/pTau231 |
| pTau231 | pTau214/pTau396 | pTau293 | pTau214/pTau396 | pTau396 | pTau214/pTau293 |
| pTau231 | pTau217/pTau293 | pTau293 | pTau217/pTau231 | pTau396 | pTau217/pTau231 |
| pTau231 | pTau217/pTau396 | pTau293 | pTau217/pTau396 | pTau396 | pTau217/pTau293 |
| pTau231 | pTau293/pTau396 | pTau293 | pTau231/pTau396 | pTau396 | pTau231/pTau293 |

TABLE 2

| Binding | Binding | Binding | Binding | Binding | Binding | Binding | Binding | Binding |
|---|---|---|---|---|---|---|---|---|
| Reagent | Reagent | Reagent | Reagent | Reagent | Reagent | Reagent | Reagent | Reagent |
| 1 | 2 | 3 | 1 | 2 | 3 | 1 | 2 | 3 |
| T175 | T181 | T212 | T205 | S214 | S293 | T231 | S293 | T212 |
| T175 | T181 | T205 | T205 | S214 | S396 | T231 | S293 | T205 |
| T175 | T181 | S214 | T205 | S214 | BE-Tau | T231 | S293 | S214 |
| T175 | T181 | T217 | T205 | S214 | T175 | T231 | S293 | T217 |
| T175 | T181 | T231 | T205 | S214 | T181 | T231 | S293 | S396 |
| T175 | T181 | S293 | T205 | S214 | T212 | T231 | S293 | BE-Tau |
| T175 | T181 | S396 | T205 | S214 | T217 | T231 | S293 | T175 |
| T175 | T181 | BE-Tau | T205 | S214 | T231 | T231 | S293 | T181 |
| T175 | T212 | T181 | T205 | T217 | S293 | T231 | S396 | T212 |
| T175 | T212 | T205 | T205 | T217 | S396 | T231 | S396 | T205 |
| T175 | T212 | S214 | T205 | T217 | BE-Tau | T231 | S396 | S214 |
| T175 | T212 | T217 | T205 | T217 | T175 | T231 | S396 | T217 |
| T175 | T212 | T231 | T205 | T217 | T181 | T231 | S396 | S293 |
| T175 | T212 | S293 | T205 | T217 | T212 | T231 | S396 | BE-Tau |
| T175 | T212 | S396 | T205 | T217 | S214 | T231 | S396 | T175 |
| T175 | T212 | BE-Tau | T205 | T217 | T231 | T231 | S396 | T181 |
| T175 | T205 | T181 | T205 | T231 | S293 | T231 | BE-Tau | T212 |
| T175 | T205 | T212 | T205 | T231 | S396 | T231 | BE-Tau | T205 |
| T175 | T205 | S214 | T205 | T231 | BE-Tau | T231 | BE-Tau | S214 |
| T175 | T205 | T217 | T205 | T231 | T175 | T231 | BE-Tau | T217 |
| T175 | T205 | T231 | T205 | T231 | T181 | T231 | BE-Tau | S293 |
| T175 | T205 | S293 | T205 | T231 | T212 | T231 | BE-Tau | S396 |
| T175 | T205 | S396 | T205 | T231 | S214 | T231 | BE-Tau | T175 |
| T175 | T205 | BE-Tau | T205 | T231 | T217 | T231 | BE-Tau | T181 |
| T175 | S214 | T181 | T205 | S293 | T231 | S293 | T175 | T212 |
| T175 | S214 | T212 | T205 | S293 | S396 | S293 | T175 | T205 |
| T175 | S214 | T205 | T205 | S293 | BE-Tau | S293 | T175 | S214 |
| T175 | S214 | T217 | T205 | S293 | T175 | S293 | T175 | T217 |
| T175 | S214 | T231 | T205 | S293 | T181 | S293 | T175 | T231 |
| T175 | S214 | S293 | T205 | S293 | T212 | S293 | T175 | S396 |
| T175 | S214 | S396 | T205 | S293 | S214 | S293 | T175 | BE-Tau |
| T175 | S214 | BE-Tau | T205 | S293 | T217 | S293 | T175 | T181 |
| T175 | T217 | T181 | T205 | S396 | T231 | S293 | T181 | T212 |
| T175 | T217 | T212 | T205 | S396 | S293 | S293 | T181 | T205 |
| T175 | T217 | T205 | T205 | S396 | BE-Tau | S293 | T181 | S214 |
| T175 | T217 | S214 | T205 | S396 | T175 | S293 | T181 | T217 |
| T175 | T217 | T231 | T205 | S396 | T181 | S293 | T181 | T231 |
| T175 | T217 | S293 | T205 | S396 | T212 | S293 | T181 | S396 |
| T175 | T217 | S396 | T205 | S396 | S214 | S293 | T181 | BE-Tau |
| T175 | T217 | BE-Tau | T205 | S396 | T217 | S293 | T181 | T175 |
| T175 | T231 | T181 | T205 | BE-Tau | T231 | S293 | T212 | T181 |
| T175 | T231 | T212 | T205 | BE-Tau | S293 | S293 | T212 | T205 |
| T175 | T231 | T205 | T205 | BE-Tau | S396 | S293 | T212 | S214 |
| T175 | T231 | S214 | T205 | BE-Tau | T175 | S293 | T212 | T217 |
| T175 | T231 | T217 | T205 | BE-Tau | T181 | S293 | T212 | T231 |
| T175 | T231 | S293 | T205 | BE-Tau | T212 | S293 | T212 | S396 |
| T175 | T231 | S396 | T205 | BE-Tau | S214 | S293 | T212 | BE-Tau |
| T175 | T231 | BE-Tau | T205 | BE-Tau | T217 | S293 | T212 | T175 |
| T175 | S293 | T181 | S214 | T175 | T231 | S293 | T205 | T181 |
| T175 | S293 | T212 | S214 | T175 | S293 | S293 | T205 | T212 |
| T175 | S293 | T205 | S214 | T175 | S396 | S293 | T205 | S214 |
| T175 | S293 | S214 | S214 | T175 | BE-Tau | S293 | T205 | T217 |
| T175 | S293 | T217 | S214 | T175 | T181 | S293 | T205 | T231 |
| T175 | S293 | T231 | S214 | T175 | T212 | S293 | T205 | S396 |
| T175 | S293 | S396 | S214 | T175 | T205 | S293 | T205 | BE-Tau |
| T175 | S293 | BE-Tau | S214 | T175 | T217 | S293 | T205 | T175 |
| T175 | S396 | T181 | S214 | T181 | T231 | S293 | S214 | T181 |
| T175 | S396 | T212 | S214 | T181 | S293 | S293 | S214 | T212 |
| T175 | S396 | T205 | S214 | T181 | S396 | S293 | S214 | T205 |
| T175 | S396 | S214 | S214 | T181 | BE-Tau | S293 | S214 | T217 |
| T175 | S396 | T217 | S214 | T181 | T175 | S293 | S214 | T231 |
| T175 | S396 | T231 | S214 | T181 | T212 | S293 | S214 | S396 |
| T175 | S396 | S293 | S214 | T181 | T205 | S293 | S214 | BE-Tau |
| T175 | S396 | BE-Tau | S214 | T181 | T217 | S293 | S214 | T175 |
| T175 | BE-Tau | T181 | S214 | T212 | T231 | S293 | T217 | T181 |
| T175 | BE-Tau | T212 | S214 | T212 | S293 | S293 | T217 | T212 |
| T175 | BE-Tau | T205 | S214 | T212 | S396 | S293 | T217 | T205 |
| T175 | BE-Tau | S214 | S214 | T212 | BE-Tau | S293 | T217 | S214 |
| T175 | BE-Tau | T217 | S214 | T212 | T175 | S293 | T217 | T231 |
| T175 | BE-Tau | T231 | S214 | T212 | T181 | S293 | T217 | S396 |
| T175 | BE-Tau | S293 | S214 | T212 | T205 | S293 | T217 | BE-Tau |
| T175 | BE-Tau | S396 | S214 | T212 | T217 | S293 | T217 | T175 |
| T181 | T175 | BE-Tau | S214 | T205 | T231 | S293 | T231 | T181 |
| T181 | T175 | T212 | S214 | T205 | S293 | S293 | T231 | T212 |
| T181 | T175 | T205 | S214 | T205 | S396 | S293 | T231 | T205 |
| T181 | T175 | S214 | S214 | T205 | BE-Tau | S293 | T231 | S214 |
| T181 | T175 | T217 | S214 | T205 | T175 | S293 | T231 | T217 |
| T181 | T175 | T231 | S214 | T205 | T181 | S293 | T231 | S396 |
| T181 | T175 | S293 | S214 | T205 | T212 | S293 | T231 | BE-Tau |
| T181 | T175 | S396 | S214 | T205 | T217 | S293 | T231 | T175 |
| T181 | T212 | BE-Tau | S214 | T217 | T231 | S293 | S396 | T181 |
| T181 | T212 | T175 | S214 | T217 | S293 | S293 | S396 | T212 |
| T181 | T212 | T205 | S214 | T217 | S396 | S293 | S396 | T205 |
| T181 | T212 | S214 | S214 | T217 | BE-Tau | S293 | S396 | S214 |
| T181 | T212 | T217 | S214 | T217 | T175 | S293 | S396 | T217 |
| T181 | T212 | T231 | S214 | T217 | T181 | S293 | S396 | T231 |
| T181 | T212 | S293 | S214 | T217 | T212 | S293 | S396 | BE-Tau |
| T181 | T212 | S396 | S214 | T217 | T205 | S293 | S396 | T175 |
| T181 | T205 | BE-Tau | S214 | T231 | T217 | S293 | BE-Tau | T181 |
| T181 | T205 | T175 | S214 | T231 | S293 | S293 | BE-Tau | T212 |
| T181 | T205 | T212 | S214 | T231 | S396 | S293 | BE-Tau | T205 |
| T181 | T205 | S214 | S214 | T231 | BE-Tau | S293 | BE-Tau | S214 |
| T181 | T205 | T217 | S214 | T231 | T175 | S293 | BE-Tau | T217 |
| T181 | T205 | T231 | S214 | T231 | T181 | S293 | BE-Tau | T231 |
| T181 | T205 | S293 | S214 | T231 | T212 | S293 | BE-Tau | S396 |
| T181 | T205 | S396 | S214 | T231 | T205 | S293 | BE-Tau | T175 |
| T181 | S214 | BE-Tau | S214 | S293 | T217 | S396 | T175 | T181 |
| T181 | S214 | T175 | S214 | S293 | T231 | S396 | T175 | T212 |
| T181 | S214 | T212 | S214 | S293 | S396 | S396 | T175 | T205 |
| T181 | S214 | T205 | S214 | S293 | BE-Tau | S396 | T175 | S214 |
| T181 | S214 | T217 | S214 | S293 | T175 | S396 | T175 | T217 |
| T181 | S214 | T231 | S214 | S293 | T181 | S396 | T175 | T231 |
| T181 | S214 | S293 | S214 | S293 | T212 | S396 | T175 | S293 |
| T181 | S214 | S396 | S214 | S293 | T205 | S396 | T175 | BE-Tau |
| T181 | T217 | BE-Tau | S214 | S396 | T217 | S396 | T181 | T175 |
| T181 | T217 | T175 | S214 | S396 | T231 | S396 | T181 | T212 |
| T181 | T217 | T212 | S214 | S396 | S293 | S396 | T181 | T205 |
| T181 | T217 | T205 | S214 | S396 | BE-Tau | S396 | T181 | S214 |
| T181 | T217 | S214 | S214 | S396 | T175 | S396 | T181 | T217 |
| T181 | T217 | T231 | S214 | S396 | T181 | S396 | T181 | T231 |
| T181 | T217 | S293 | S214 | S396 | T212 | S396 | T181 | S293 |
| T181 | T217 | S396 | S214 | S396 | T205 | S396 | T181 | BE-Tau |
| T181 | T231 | BE-Tau | S214 | BE-Tau | T217 | S396 | T212 | T175 |
| T181 | T231 | T175 | S214 | BE-Tau | T231 | S396 | T212 | T181 |
| T181 | T231 | T212 | S214 | BE-Tau | S293 | S396 | T212 | T205 |
| T181 | T231 | T205 | S214 | BE-Tau | S396 | $396 | T212 | S214 |
| T181 | T231 | S214 | S214 | BE-Tau | T175 | S396 | T212 | T217 |
| T181 | T231 | T217 | S214 | BE-Tau | T181 | S396 | T212 | T231 |
| T181 | T231 | S293 | S214 | BE-Tau | T212 | S396 | T212 | S293 |
| T181 | T231 | S396 | S214 | BE-Tau | T205 | S396 | T212 | BE-Tau |
| T181 | S293 | BE-Tau | T217 | T175 | S214 | S396 | T205 | T175 |
| T181 | S293 | T175 | T217 | T175 | T231 | S396 | T205 | T181 |
| T181 | S293 | T212 | T217 | T175 | S293 | S396 | T205 | T212 |
| T181 | S293 | T205 | T217 | T175 | S396 | S396 | T205 | S214 |
| T181 | S293 | S214 | T217 | T175 | BE-Tau | S396 | T205 | T217 |
| T181 | S293 | T217 | T217 | T175 | T181 | S396 | T205 | T231 |
| T181 | S293 | T231 | T217 | T175 | T212 | S396 | T205 | S293 |
| T181 | S293 | S396 | T217 | T175 | T205 | S396 | T205 | BE-Tau |
| T181 | S396 | BE-Tau | T217 | T181 | S214 | S396 | S214 | T175 |
| T181 | S396 | T175 | T217 | T181 | T231 | S396 | S214 | T181 |
| T181 | S396 | T212 | T217 | T181 | S293 | S396 | S214 | T212 |
| T181 | S396 | T205 | T217 | T181 | S396 | S396 | S214 | T205 |
| T181 | S396 | S214 | T217 | T181 | BE-Tau | S396 | S214 | T217 |
| T181 | S396 | T217 | T217 | T181 | T175 | S396 | S214 | T231 |
| T181 | S396 | T231 | T217 | T181 | T212 | S396 | S214 | S293 |
| T181 | S396 | S293 | T217 | T181 | T205 | S396 | S214 | BE-Tau |
| T181 | BE-Tau | S396 | T217 | T212 | S214 | S396 | T217 | T175 |
| T181 | BE-Tau | T175 | T217 | T212 | T231 | S396 | T217 | T181 |
| T181 | BE-Tau | T212 | T217 | T212 | S293 | S396 | T217 | T212 |
| T181 | BE-Tau | T205 | T217 | T212 | S396 | S396 | T217 | T205 |
| T181 | BE-Tau | S214 | T217 | T212 | BE-Tau | S396 | T217 | S214 |
| T181 | BE-Tau | T217 | T217 | T212 | T175 | S396 | T217 | T231 |
| T181 | BE-Tau | T231 | T217 | T212 | T181 | S396 | T217 | S293 |
| T181 | BE-Tau | S293 | T217 | T212 | T205 | S396 | T217 | BE-Tau |
| T212 | T175 | S396 | T217 | T205 | S214 | S396 | T231 | T175 |
| T212 | T175 | BE-Tau | T217 | T205 | T231 | S396 | T231 | T181 |
| T212 | T175 | T181 | T217 | T205 | S293 | S396 | T231 | T212 |
| T212 | T175 | T205 | T217 | T205 | S396 | S396 | T231 | T205 |
| T212 | T175 | S214 | T217 | T205 | BE-Tau | S396 | T231 | S214 |
| T212 | T175 | T217 | T217 | T205 | T175 | S396 | T231 | T217 |
| T212 | T175 | T231 | T217 | T205 | T181 | S396 | T231 | S293 |
| T212 | T175 | S293 | T217 | T205 | T212 | S396 | T231 | BE-Tau |
| T212 | T181 | S396 | T217 | S214 | T205 | S396 | S293 | T175 |
| T212 | T181 | BE-Tau | T217 | S214 | T231 | S396 | S293 | T181 |
| T212 | T181 | T175 | T217 | S214 | S293 | S396 | S293 | T212 |
| T212 | T181 | T205 | T217 | S214 | S396 | S396 | S293 | T205 |
| T212 | T181 | S214 | T217 | S214 | BE-Tau | S396 | S293 | S214 |
| T212 | T181 | T217 | T217 | S214 | T175 | S396 | S293 | T217 |
| T212 | T181 | T231 | T217 | S214 | T181 | S396 | S293 | T231 |
| T212 | T181 | S293 | T217 | S214 | T212 | S396 | S293 | BE-Tau |
| T212 | T205 | S396 | T217 | T231 | T205 | S396 | BE-Tau | T175 |
| T212 | T205 | BE-Tau | T217 | T231 | S214 | S396 | BE-Tau | T181 |
| T212 | T205 | T175 | T217 | T231 | S293 | S396 | BE-Tau | T212 |
| T212 | T205 | T181 | T217 | T231 | S396 | S396 | BE-Tau | T205 |
| T212 | T205 | S214 | T217 | T231 | BE-Tau | S396 | BE-Tau | S214 |
| T212 | T205 | T217 | T217 | T231 | T175 | S396 | BE-Tau | T217 |
| T212 | T205 | T231 | T217 | T231 | T181 | S396 | BE-Tau | T231 |
| T212 | T205 | S293 | T217 | T231 | T212 | S396 | BE-Tau | S293 |
| T212 | S214 | S396 | T217 | S293 | T205 | BE-Tau | T175 | S396 |
| T212 | S214 | BE-Tau | T217 | S293 | S214 | BE-Tau | T175 | T181 |
| T212 | S214 | T175 | T217 | S293 | T231 | BE-Tau | T175 | T212 |
| T212 | S214 | T181 | T217 | S293 | S396 | BE-Tau | T175 | T205 |
| T212 | S214 | T205 | T217 | S293 | BE-Tau | BE-Tau | T175 | S214 |
| T212 | S214 | T217 | T217 | S293 | T175 | BE-Tau | T175 | T217 |
| T212 | S214 | T231 | T217 | S293 | T181 | BE-Tau | T175 | T231 |
| T212 | S214 | S293 | T217 | S293 | T212 | BE-Tau | T175 | S293 |
| T212 | T217 | S396 | T217 | S396 | T205 | BE-Tau | T181 | S396 |
| T212 | T217 | BE-Tau | T217 | S396 | S214 | BE-Tau | T181 | T175 |
| T212 | T217 | T175 | T217 | S396 | T231 | BE-Tau | T181 | T212 |
| T212 | T217 | T181 | T217 | S396 | S293 | BE-Tau | T181 | T205 |
| T212 | T217 | T205 | T217 | S396 | BE-Tau | BE-Tau | T181 | S214 |
| T212 | T217 | S214 | T217 | S396 | T175 | BE-Tau | T181 | T217 |
| T212 | T217 | T231 | T217 | S396 | T181 | BE-Tau | T181 | T231 |
| T212 | T217 | S293 | T217 | S396 | T212 | BE-Tau | T181 | S293 |
| T212 | T231 | S396 | T217 | BE-Tau | T205 | BE-Tau | T212 | S396 |
| T212 | T231 | BE-Tau | T217 | BE-Tau | S214 | BE-Tau | T212 | T175 |
| T212 | T231 | T175 | T217 | BE-Tau | T231 | BE-Tau | T212 | T181 |
| T212 | T231 | T181 | T217 | BE-Tau | S293 | BE-Tau | T212 | T205 |
| T212 | T231 | T205 | T217 | BE-Tau | S396 | BE-Tau | T212 | S214 |
| T212 | T231 | S214 | T217 | BE-Tau | T175 | BE-Tau | T212 | T217 |
| T212 | T231 | T217 | T217 | BE-Tau | T181 | BE-Tau | T212 | T231 |
| T212 | T231 | S293 | T217 | BE-Tau | T212 | BE-Tau | T212 | S293 |
| T212 | S293 | S396 | T231 | T175 | T205 | BE-Tau | T205 | S396 |
| T212 | S293 | BE-Tau | T231 | T175 | S214 | BE-Tau | T205 | T175 |
| T212 | S293 | T175 | T231 | T175 | T217 | BE-Tau | T205 | T181 |
| T212 | S293 | T181 | T231 | T175 | S293 | BE-Tau | T205 | T212 |
| T212 | S293 | T205 | T231 | T175 | S396 | BE-Tau | T205 | S214 |
| T212 | S293 | S214 | T231 | T175 | BE-Tau | BE-Tau | T205 | T217 |
| T212 | S293 | T217 | T231 | T175 | T181 | BE-Tau | T205 | T231 |
| T212 | S293 | T231 | T231 | T175 | T212 | BE-Tau | T205 | S293 |
| T212 | S396 | S293 | T231 | T181 | T205 | BE-Tau | S214 | S396 |
| T212 | S396 | BE-Tau | T231 | T181 | S214 | BE-Tau | S214 | T175 |
| T212 | S396 | T175 | T231 | T181 | T217 | BE-Tau | S214 | T181 |
| T212 | S396 | T181 | T231 | T181 | S293 | BE-Tau | S214 | T212 |
| T212 | S396 | T205 | T231 | T181 | S396 | BE-Tau | S214 | T205 |
| T212 | S396 | S214 | T231 | T181 | BE-Tau | BE-Tau | S214 | T217 |
| T212 | S396 | T217 | T231 | T181 | T175 | BE-Tau | S214 | T231 |
| T212 | S396 | T231 | T231 | T181 | T212 | BE-Tau | S214 | S293 |
| T212 | BE-Tau | S293 | T231 | T212 | T205 | BE-Tau | T217 | S396 |
| T212 | BE-Tau | S396 | T231 | T212 | S214 | BE-Tau | T217 | T175 |
| T212 | BE-Tau | T175 | T231 | T212 | T217 | BE-Tau | T217 | T181 |
| T212 | BE-Tau | T181 | T231 | T212 | S293 | BE-Tau | T217 | T212 |
| T212 | BE-Tau | T205 | T231 | T212 | S396 | BE-Tau | T217 | T205 |
| T212 | BE-Tau | S214 | T231 | T212 | BE-Tau | BE-Tau | T217 | S214 |
| T212 | BE-Tau | T217 | T231 | T212 | T175 | BE-Tau | T217 | T231 |
| T212 | BE-Tau | T231 | T231 | T212 | T181 | BE-Tau | T217 | S293 |
| T205 | T175 | S293 | T231 | T205 | T212 | BE-Tau | T231 | S396 |
| T205 | T175 | S396 | T231 | T205 | S214 | BE-Tau | T231 | T175 |
| T205 | T175 | BE-Tau | T231 | T205 | T217 | BE-Tau | T231 | T181 |
| T205 | T175 | T181 | T231 | T205 | S293 | BE-Tau | T231 | T212 |
| T205 | T175 | T212 | T231 | T205 | S396 | BE-Tau | T231 | T205 |
| T205 | T175 | S214 | T231 | T205 | BE-Tau | BE-Tau | T231 | S214 |
| T205 | T175 | T217 | T231 | T205 | T175 | BE-Tau | T231 | T217 |
| T205 | T175 | T231 | T231 | T205 | T181 | BE-Tau | T231 | S293 |
| T205 | T181 | S293 | T231 | S214 | T212 | BE-Tau | S293 | S396 |
| T205 | T181 | S396 | T231 | S214 | T205 | BE-Tau | S293 | T175 |
| T205 | T181 | BE-Tau | T231 | S214 | T217 | BE-Tau | S293 | T181 |
| T205 | T181 | T175 | T231 | S214 | S293 | BE-Tau | S293 | T212 |
| T205 | T181 | T212 | T231 | S214 | S396 | BE-Tau | S293 | T205 |
| T205 | T181 | S214 | T231 | S214 | BE-Tau | BE-Tau | S293 | S214 |
| T205 | T181 | T217 | T231 | S214 | T175 | BE-Tau | S293 | T217 |
| T205 | T181 | T231 | T231 | S214 | T181 | BE-Tau | S293 | T231 |
| T205 | T212 | S293 | T231 | T217 | T212 | BE-Tau | S396 | S293 |
| T205 | T212 | S396 | T231 | T217 | T205 | BE-Tau | S396 | T175 |
| T205 | T212 | BE-Tau | T231 | T217 | S214 | BE-Tau | S396 | T181 |
| T205 | T212 | T175 | T231 | T217 | S293 | BE-Tau | S396 | T212 |
| T205 | T212 | T181 | T231 | T217 | S396 | BE-Tau | S396 | T205 |
| T205 | T212 | S214 | T231 | T217 | BE-Tau | BE-Tau | S396 | S214 |
| T205 | T212 | T217 | T231 | T217 | T175 | BE-Tau | S396 | T217 |
| T205 | T212 | T231 | T231 | T217 | T181 | BE-Tau | S396 | T231 |

### Brain-Associated Tau Polypeptide

The terms “brain-associated tau,” “brain-derived tau,” “brain-associated or brain-derived epitope,” or “brain-specific tau,” are understood to have the same meaning and relate to a tau protein or polypeptide sequence found in brain tissue, the Central Nervous System (CNS) and/or cerebrospinal fluid (CSF). In embodiments, the present disclosure provides a method of detecting and/or quantifying brain-associated tau.

The protein structure of tau in the Central Nervous System (CNS) and Peripheral Nervous System (PNS) have fundamental differences in splice variants: while there are six tau isoforms of varying lengths in the adult human brain, the main form of tau in the PNS is distinguishable from these isoforms by the presence of a large peptide insert resulting from the transcription of an extra exon (exon 4a) of the MAPT gene. Since CSF t-tau, but not plasma t-tau, agrees with PET and neuropathological evidence of Alzheimer's disease, it is plausible that the tau forms measured by the blood assay originate, to a large extent, from non-CNS sources. Tau in the adult human brain has six isoforms between 352 and 441 amino acids long. However, tau in peripheral tissues-including the liver, kidney, heart and pancreas—is predominantly of the high molecular weight isoform including the exon 4a insert. A recent study estimated that only a fifth of the signal measured by plasma t-tau is brain-derived while the remainder originates from peripheral sources. As a result, plasma t-tau tends to show promising diagnostic function largely in disorders with acute increases in CNS tau production and release, including traumatic brain injury and acute stroke following cardiac arrest. The disclosure provides a blood-based biomarker assay for detecting neurodegeneration specific to Alzheimer's disease. In embodiments, the present disclosure provides an immunoassay that selectively targets brain-associated tau.

The present disclosure herein provides a method of detecting tau and p-tau, comprising an immunoassay to selectively measure brain-associated tau in blood by binding and targeting tau isoforms originating from the brain, or at least associated and derived from the brain.

In embodiments, the tau or p-tau in the present disclosure comprises a brain-associated tau polypeptide wherein the brain-associated tau polypeptide comprises at least 80%, at least 90%, at least 95%, at least 99%, or 100% sequence identity to SEQ ID NO: 2 (AGHVTQARMVSK) or SEQ ID NO: 3 (HVTQARMV). In embodiments, the tau or p-tau in the present disclosure comprises a brain-associated Tau441 epitope.

In embodiments, the methods herein are capable of detecting brain-associated tau. Brain-associated tau is a distinct type of tau with unique sites or epitopes, and/or unique phosphorylation patterns, as compared to tau derived from other regions. In embodiments, the present disclosure provides a method of detecting, quantifying, or both, p-tau in a biological sample, wherein the p-tau comprises a brain-associated tau polypeptide sequence, comprising: (a) contacting the biological sample with: (i) a capture reagent that binds a phosphorylated tau site, or a non-phosphorylated tau site; (ii) a first detection reagent that binds a phosphorylated tau site, or a non-phosphorylated tau site; and (iii) a second detection reagent that binds a phosphorylated tau site, or a non-phosphorylated tau site; (b) forming a complex comprising the capture reagent, the p-tau, the first detection reagent and the second detection reagent; and (c) detecting the complex, thereby detecting the p-tau; or (d) measuring an amount of the complex, thereby quantifying an amount of the p-tau, wherein at least two of the capture reagent, first detection reagent, and second detection reagent binds a phosphorylated tau site.

In embodiments, the present disclosure provides a method of detecting, quantifying, or both, p-tau in a biological sample, wherein the p-tau comprises a brain-associated tau polypeptide sequence, comprising: (a) contacting the biological sample with: (i) a capture reagent that binds a phosphorylated tau site, or a non-phosphorylated tau site; (ii) a first detection reagent that binds a phosphorylated tau site, or a non-phosphorylated tau site; and (iii) a second detection reagent that binds a phosphorylated tau site, or a non-phosphorylated tau site; (b) forming a complex comprising the capture reagent, the p-tau, the first detection reagent and the second detection reagent; and (c) detecting the complex, thereby detecting the p-tau; or (d) measuring an amount of the complex, thereby quantifying an amount of the p-tau, wherein at least one of the capture reagent, first detection reagent, and second detection reagent binds a brain-associated tau polypeptide site.

### Binding Reagent

As used herein, the term “binding reagent” refers to a substance that specifically binds a specified target substance. A binding reagent is understood to “specifically bind” to a target substance when the binding reagent binds the target substance with sufficient preference such that the target substance is detected and/or measured, e.g., by the methods described herein. The term “specifically bind” and variants thereof, e.g., “specific binding,” does not necessarily imply that there is no other substance to which the binding reagent binds. The portion of a target substance that specifically interacts with the binding reagent may be referred to as a “binding region.” A target substance may contain a single binding region or more than one binding regions.

Non-limiting examples of binding reagents include proteins, nucleic acids, carbohydrates, polysaccharides, glycoproteins, lipids, lipoproteins, toxins, drugs, hormones, steroids, nutrients, metabolites, antigens, epitopes, any derivatives or modifications of the foregoing substances, or any complex including one or more of the foregoing substances, or any combinations thereof. Common specific examples of binding reagents include antibodies, aptamers, receptors, and avidins. The portion of a target substance that specifically interacts with binding reagent may be referred to as a “binding site.” In embodiments, the target substance is tau, and the binding site comprises a phosphorylated or non-phosphorylated site. A target substance may include a single binding site or more than one binding site. Binding sites include polypeptide elements, e.g., amino acid sequences, or non-polypeptide elements, e.g., nucleic acids, chemical groups (e.g., alkyl or acetyl groups on amino acids and/or nucleotides), carbohydrates, or other small molecules and substances. In embodiments, a binding reagent of the present disclosure includes a polypeptide, an antibody or antigen-binding fragment thereof, an antigen, a ligand, a receptor, an oligonucleotide, a hapten, an epitope, a mimotope, or an aptamer. For example, the binding reagent may include an oligonucleotide, and the target substance may include a complementary oligonucleotide.

In embodiments, a binding reagent of the present disclosure includes a polypeptide. As used herein, the terms “polypeptide” refers to a polymer of amino acid monomers linked by amide bonds (i.e., peptide bonds). The term “polypeptide” refers to any chain or chains of amino acids and does not suggest a specific length of the chain. Dipeptides, tripeptides, oligopeptides, “protein,” “amino acid chain,” and any other term used to refer to a chain or chains of amino acids are encompassed by and used interchangeably with the term “polypeptide.” The polypeptides of the present disclosure may include one or more modifications (e.g., post-translational modifications (PTMs)), including without limitation, glycosylation, acetylation, phosphorylation, amidation, derivatization by protecting and/or blocking groups, proteolytic cleavage, or incorporation of non-naturally occurring amino acids. A polypeptide may be derived from a natural biological source or produced by recombinant technology, but is not necessarily translated by a designated nucleic acid. For example, a polypeptide may be produced by partial or total chemical synthesis, or other techniques known to one of skill in the art.

In embodiments, the binding reagent of the present disclosure includes an antibody or an antibody fragment thereof.

In embodiments, the present disclosure provides a method of detecting, quantifying, or both, tau in a biological sample, wherein the tau comprises a plurality of epitopes, the method comprising: (a) contacting the biological sample with a plurality of binding reagents comprising at least three binding reagents, wherein the first binding reagent binds to a first epitope, the second binding reagent binds to a second epitope, and the third binding reagent binds to a third epitope; (b) forming a complex comprising the plurality of binding reagents and the tau; and (c) detecting the complex, thereby detecting the tau; or (d) measuring an amount of the complex, thereby quantifying an amount of the tau.

In embodiments, each binding reagent of the plurality of binding reagents independently comprises an antibody, antigen, ligand, receptor, oligonucleotide, hapten, epitope, mimitope, or aptamer. In embodiments, each binding reagent of the plurality of binding reagents comprises an antibody or antigen-binding fragment thereof.

In embodiments, the first binding reagent binds to a site in the first epitope capable of being phosphorylated. In embodiments, the first binding reagent binds to the phosphorylated site of the first epitope or binds to the non-phosphorylated site of the first epitope. In embodiments, the second binding reagent binds to a site in the second epitope capable of being phosphorylated. In embodiments, the second binding reagent binds to the phosphorylated site of the second epitope or binds to the non-phosphorylated site of the second epitope. In embodiments, the third binding reagent binds to a site in the third epitope capable of being phosphorylated. In embodiments, the third binding reagent binds to the phosphorylated site of the third epitope or binds to the non-phosphorylated site of the third epitope.

In embodiments, the method disclosed herein further comprises a fourth binding reagent. In embodiments, the fourth binding reagent binds to a fourth epitope. In embodiments, the fourth binding reagent binds to a site in the fourth epitope capable of being phosphorylated. In embodiments, the fourth binding reagent binds to the phosphorylated site of the fourth epitope or binds to the non-phosphorylated site of the fourth epitope.

In embodiments, the fourth binding reagent binds to a same epitope as one of the first, second, or third binding reagents, but with a different phosphorylation state. In embodiments, the first binding reagent binds to a first epitope comprising a phosphorylated first site, and the fourth binding reagent binds to the non-phosphorylated first site. In embodiments, the second binding reagent binds to a second epitope comprising a second phosphorylated site and the fourth binding reagent binds to the non-phosphorylated second site. In embodiments, the third binding reagent binds to a third epitope comprising a phosphorylated third site, and the fourth binding reagent binds to the non-phosphorylated third site.

In embodiments, the first binding reagent binds to any one of the eighty-five serine (S), threonine (T), and tyrosine (Y) tau sites capable of being phosphorylated. In embodiments, the first binding reagent binds to any one of the other tau sites that are not one of the eighty-five serine (S), threonine (T), and tyrosine (Y) tau sites capable of being phosphorylated. In embodiments, the first binding reagent binds to a brain-associated tau epitope.

In embodiments, the second binding reagent binds to any one of the eighty-five serine (S), threonine (T), and tyrosine (Y) tau sites capable of being phosphorylated. In embodiments, the second binding reagent binds to any one of the other tau sites that are not one of the eighty-five serine (S), threonine (T), and tyrosine (Y) tau sites capable of being phosphorylated. In embodiments, the second binding reagent binds to a brain-associated tau epitope.

In embodiments, the third binding reagent binds to any one of the eighty-five serine (S), threonine (T), and tyrosine (Y) tau sites capable of being phosphorylated. In embodiments, the third binding reagent binds to any one of the other tau sites that are not one of the eighty-five serine (S), threonine (T), and tyrosine (Y) tau sites capable of being phosphorylated. In embodiments, the third binding reagent binds to a brain-associated tau epitope.

In embodiments, the fourth binding reagent binds to any one of the eighty-five serine (S), threonine (T), and tyrosine (Y) tau sites capable of being phosphorylated. In embodiments, the fourth binding reagent binds to any one of the other tau sites that are not one of the eighty-five serine (S), threonine (T), and tyrosine (Y) tau sites capable of being phosphorylated. In embodiments, the fourth binding reagent binds to a brain-associated tau epitope.

In embodiments, the first binding reagent is a capture reagent. In embodiments, the second binding reagent is a first detection reagent. In embodiments, the third binding reagent is a second detection reagent. In embodiments, the fourth binding reagent is a third detection reagent. In embodiments, the fourth binding reagent is a second capture reagent.

In embodiments, at least one of the binding reagents binds to total tau. In embodiments, at least one of the binding reagents binds to a brain-associated epitope. In embodiments, the brain-associated epitope is a part of the Tau441 isoform of SEQ ID NO: 1. In embodiments, the brain-associated epitope comprises SEQ ID NO: 2 or 3.

In embodiments, at least one of the binding reagents is immobilized to a surface. In embodiments, the one or more binding reagent immobilized to the surface is a capture reagent. In embodiments, each binding reagent comprises an oligonucleotide sequence.

### Capture Reagent

The methods of the present disclosure utilize a capture reagent that binds to the analyte of interest, e.g., tau, p-tau (including pTau217) or non-phosphorylated tau. In embodiments, the capture reagent of the present disclosure binds to the p-tau or non-phosphorylated tau. In embodiments, the capture reagent is capable of binding single picogram levels of tau in a sample.

In embodiments, the capture reagent is an antibody, antigen, ligand, receptor, oligonucleotide, hapten, epitope, mimotope, or an aptamer. In embodiments, the capture reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the capture reagent comprises at least one heavy or light chain complementarity determining region (CDR) of an antibody. In embodiments, the capture reagent comprises at least two CDRs from one or more antibodies. In embodiments, the capture reagent is an antibody or antigen-binding fragment thereof. In embodiments, the capture reagent binds tau. In embodiments, the capture reagent binds total tau. In embodiments, the capture reagent binds tau. In embodiments, the capture reagent is capable of binding to non-phosphorylated tau. In embodiments, the capture reagent is capable of binding to p-tau. In embodiments, the capture reagent is capable of binding both non-phosphorylated tau and p-tau. In embodiments, the capture reagent is capable of binding p-tau at any amino acid position, site or residue of any one of SEQ ID NOs: 1-9. In embodiments, the capture reagent is capable of binding p-tau at any phosphorylated amino acid position, site or residue of any one of SEQ ID NOs: 1-9. In embodiments, the capture reagent is capable of binding p-tau that is phosphorylated at T175, T181, T205, T212, S214, T217, cis T231, trans T231, S293, S396, or a combination thereof of SEQ ID NO: 1. In embodiments, the capture reagent is capable of binding p-tau or tau at L243. In embodiments, the capture reagent is capable of binding to p-tau that is phosphorylated at T217 and does not bind non-phosphorylated T217. In embodiments, the capture reagent binds pTau181, pTau205, pTau212, pTau217 or pTau231, and does not bind non-phosphorylated tau.

In embodiments, the capture reagent is immobilized on a surface prior to being contacted with the sample. In embodiments, the capture reagent is immobilized on the surface simultaneously or substantially simultaneously as being contacted with the sample. In embodiments, the capture reagent is contacted with the sample, then immobilized on the surface. In embodiments, the capture reagent is immobilized on the surface.

In embodiments, the capture reagent includes a capture reagent that is directly attached to the support surface. In embodiments, the capture reagent includes a targeting reagent that is capable of binding to a targeting reagent complement that is attached to the support surface. In embodiments, the capture reagent is immobilized on the support surface through a linker. In embodiments, the capture reagent is attached to the support surface through a pair of complementary oligonucleotides, in which one oligonucleotide is attached to the support surface and the other is attached to the capture reagent. In one aspect, the complementary oligonucleotides include a restriction site. In one aspect, the restriction site in the complementary oligonucleotides is cleaved by a restriction endonuclease to release the capture reagent from the support surface.

In embodiments, the methods of the present disclosure utilize two capture reagents, i.e., a first capture reagent and a second capture reagent, that bind to two epitopes of the analyte of interest, e.g., pTau (including pTau181 and pTau217) or non-phosphorylated tau, referred to herein as a “dual-capture” assay. In embodiments, the two capture reagents of a dual-capture assay bind synergistically to the pTau or non-phosphorylated tau. As used herein, “synergistic binding” refers to a higher binding affinity when using two binding reagents compared with the sum of each binding reagent's individual binding affinity. In embodiments, the capture reagents of a dual-capture assay bind the analyte of interest, e.g., pTau (including pTau181 and pTau217) or non-phosphorylated tau, with higher affinity compared with a single capture reagent. In embodiments, the capture reagents of a dual-capture assay bind the analyte of interest, e.g., pTau (including pTau181 and pTau217) or non-phosphorylated tau, with greater specificity compared with a single capture reagent. In embodiments, a dual-capture assay has improved sensitivity and/or specificity relative to an assay utilizing a single capture reagent. In embodiments, a dual-capture assay is capable of detecting single picogram levels of pTau181, pTau217, and/or total tau in a sample. In embodiments, a dual-capture assay has a lower limit of quantitation (LLOQ) of less than 20 μg/mL, less than 18 μg/mL, less than 15 μg/mL, less than 12 μg/mL, less than 10 μg/mL, less than 5 μg/mL, or less than 1 μg/mL of pTau181, pTau217, and/or total tau. In embodiments, at least one of the capture reagents of a dual-capture assay binds to total tau. In embodiments, the first capture reagent and the second capture reagent bind to total tau. In embodiments, at least one of the capture reagents of a dual-capture assay binds to a brain-associated tau epitope. In embodiments, the first capture reagent and the second capture reagent bind to a brain-associated tau epitope. In embodiments, the first capture reagent and the second capture reagent bind to two different brain-associated tau epitopes shared among brain-associated tau splice variants. In embodiments, the first capture reagent and/or the second capture reagent bind to any epitope or site in any one of SEQ ID NO: 1-9. In embodiments, the first capture reagent and/or the second capture reagent bind to any phosphorylated epitope or site in any one of SEQ ID NO: 1-9.In embodiments, the first capture reagent and/or the second capture reagent bind to any epitope or site in SEQ ID NO: 2 or 3. In embodiments, the first capture reagent and/or the second capture reagent bind to any phosphorylated epitope or site in SEQ ID NO: 2 or 3.

In embodiments, the first capture reagent is an antibody, antigen, ligand, receptor, oligonucleotide, hapten, epitope, mimotope, or an aptamer. In embodiments, the first capture reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the first capture reagent comprises at least one heavy or light chain complementarity determining region (CDR) of an antibody. In embodiments, the first capture reagent comprises at least two CDRs from one or more antibodies. In embodiments, the first capture reagent is an antibody or antigen-binding fragment thereof. In embodiments, the first capture reagent binds tau. In embodiments, the first capture reagent is capable of binding to non-phosphorylated tau. In embodiments, the first capture reagent is capable of binding to pTau. In embodiments, the first capture reagent is capable of binding to both non-phosphorylated tau and pTau. In embodiments, the first capture reagent is capable of binding to pTau that is phosphorylated at T175, T181, T205, T212, 5214, T217, cis T231, trans T231, S293, S396, or a combination thereof. In embodiments, the method detects pTau181, and the first capture reagent is capable of binding to pTau that is phosphorylated at T181. In embodiments, the method detects pTau217, and the first capture reagent is capable of specifically binding to pTau217.

In embodiments, the second capture reagent is an antibody, antigen, ligand, receptor, oligonucleotide, hapten, epitope, mimotope, or an aptamer. In embodiments, the second capture reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the second capture reagent comprises at least one heavy or light chain complementarity determining region (CDR) of an antibody. In embodiments, the second capture reagent comprises at least two CDRs from one or more antibodies. In embodiments, the second capture reagent is an antibody or antigen-binding fragment thereof. In embodiments, the second capture reagent binds tau. In embodiments, the second capture reagent is capable of binding to non-phosphorylated tau. In embodiments, the second capture reagent is capable of binding to pTau. In embodiments, the second capture reagent is capable of binding to both non-phosphorylated tau and pTau. In embodiments, the second capture reagent is capable of binding to pTau that is phosphorylated at T175, T181, T205, T212, 5214, T217, cis T231, trans T231, S293, S396, or any combination thereof of SEQ ID NO: 1, or a combination thereof. In embodiments, the method detects pTau181, and the second capture reagent is capable of binding to pTau that is phosphorylated at T181. In embodiments, the method detects pTau217, and the second capture reagent is capable of specifically binding to tau.

In embodiments, each of the first and second capture reagents is an antibody or antigen-binding fragment thereof. In embodiments, each of the first and second capture reagent binds tau. In embodiments, the first and second capture reagents are capable of binding non-phosphorylated tau. In embodiments, the first and second capture reagents are capable of binding to pTau. In embodiments, the first and second capture reagents are capable of binding non-phosphorylated tau and pTau. In embodiments, the method detects pTau181, and the first and second capture reagents are capable of binding to pTau phosphorylated at T175, T181, T205, T212, 5214, T217, cis T231, trans T231, 5293, 5396, or any combination thereof of SEQ ID NO: 1, or a combination thereof.

In embodiments, the first and/or second capture reagents are immobilized on a surface prior to being contacted with the sample. In embodiments, one or both of the first and second capture reagents are immobilized on the surface simultaneously or substantially simultaneously as being contacted with the sample. In embodiments, the first and second capture reagents are contacted with the sample, then one or both of the first and second capture reagents are immobilized on the surface. In embodiments, the first and second capture reagents are immobilized on the surface. Immobilization of capture reagents onto the surface are further described herein.

In embodiments, the method comprises a first capture reagent, a first detection reagent, and a second detection reagent, which bind to first, second, and third sites as described herein. In embodiments, the method further comprises a second capture reagent. In embodiments, the first capture reagent binds to a phosphorylated state of the first site or binds to a non-phosphorylated state of the first site. In embodiments, the first detection reagent binds to a phosphorylated state of the second site or binds to a non-phosphorylated state of the second site. In embodiments, the second detection reagent binds to a phosphorylated state of the third site or binds to a non-phosphorylated state of the third site. In embodiments, the method optionally further comprises a third detection reagent that binds to a non-phosphorylated state or a phosphorylated state of a fourth site. In embodiments, the second capture reagent binds to a phosphorylated state of a fourth or fifth site or binds to a non-phosphorylated state of a fourth or fifth site. In embodiments, the first capture reagent binds to T217. In embodiments, the first capture reagent binds to the phosphorylated state of T217 and the second capture reagent binds to any site of tau. In embodiments, the first capture reagent binds to the phosphorylated state of T217 and the second capture reagent binds to any one of the eighty-five serine (S), threonine (T), and tyrosine (Y) tau sites capable of being phosphorylated. In embodiments, the first or second capture reagent binds to any tau site. In embodiments, the first or second capture reagent binds to a tau site rarely or never phosphorylated. In embodiments, the first or second capture reagent binds to a tau site commonly phosphorylated.

### Detection Reagent

In embodiments, the method comprises contacting the sample with a detection reagent, thereby forming a complex comprising the capture reagent, the tau or p-tau and the detection reagent. In embodiments, the method comprises detecting and/or quantifying the complex, thereby detecting and/or quantifying tau or p-tau. In embodiments, the method further comprises binding the complex to a surface prior to the detecting and/or quantification. In embodiments, the complex is bound to the surface via immobilization of the capture reagent to the surface. In embodiments, the complex is formed on the surface. In embodiments, the complex is formed in solution, then bound to the surface. Formation of the complex and surfaces are further described herein.

In embodiments, the detection reagent is an antibody, antigen, ligand, receptor, oligonucleotide, hapten, epitope, mimotope, or an aptamer. In embodiments, the detection reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the detection reagent comprises at least one heavy or light chain complementarity determining region (CDR) of an antibody. In embodiments, the detection reagent comprises at least two CDRs from one or more antibodies. In embodiments, the detection reagent is an antibody or antigen-binding fragment thereof.

In embodiments, the detection reagent is capable of binding p-tau at any amino acid position or residue of SEQ ID NOs: 1-9. In embodiments, the detection reagent is capable of binding p-tau at any phosphorylated amino acid position, site or residue of SEQ ID NOs: 1-9. In embodiments where the method detects and/or quantifies tau or p-tau, the detection reagent specifically binds pTau231. In embodiments where the method detects and/or quantifies tau or p-tau, the detection reagent binds pTau231 and does not bind non-phosphorylated tau. In embodiments where the method detects and/or quantifies tau or p-tau, the detection reagent binds pTau231 and does not bind p-tau that is not phosphorylated at amino acid position T231.

In embodiments where the method detects and/or quantifies tau or p-tau, the detection reagent is capable of binding non-phosphorylated tau and/or p-tau, for example, p-tau that is phosphorylated at any one of the amino acid positions or sites: T175, T181, T205, T212, T205, S214, T217, cis T231, trans T231, S293, S396, or a combination thereof with reference to SEQ ID NO: 1. In embodiments, the detection reagent binds pTau181, pTau205, pTau212, pTau217, or pTau231, and does not bind non-phosphorylated tau. In embodiments, the detection reagent is capable of binding p-tau at L243.

In embodiments, the detection reagent comprises a detectable label. In embodiments, the detecting step of the method comprises measuring the amount of the detectable label. In embodiments where the complex is bound to a surface, the method comprises measuring the amount of detectable label on the surface. In embodiments, the detectable label is capable of being measured by light scattering, optical absorbance, fluorescence, chemiluminescence, electrochemiluminescence (ECL), bioluminescence, phosphorescence, radioactivity, magnetic field, or combinations thereof. In embodiments, the detectable label comprises one or more ECL labels, and the detecting comprises measuring an ECL signal. Exemplary ECL labels and methods of measuring ECL signals are described, e.g., in U.S. Pat. Nos. 5,714,089; 6,136,268; 6,316,607; 6,468,741; 6,479,233; 6,808,939; and 9,499,573. In embodiments, the amount of measured detectable label, e.g., the amount of measured ECL signal, is used to determine the amount of tau, p-tau, or total tau, present in the sample.

In embodiments, the detection reagent comprises a nucleic acid probe. In embodiments, the detecting step of the method comprises: (i) extending the nucleic acid probe to form an extended sequence; and (ii) measuring the amount of the extended sequence. In embodiments, the complex is bound to a surface prior to the detecting, and the surface further comprises an anchoring reagent. In embodiments, the detecting step of the method comprises: (i) extending the nucleic acid probe to form an extended sequence comprising an anchoring region that binds to the anchoring reagent; (ii) binding the extended sequence to the anchoring reagent; and (iii) measuring the amount of extended sequence bound to the surface. In embodiments, the amount of measured extended sequence, e.g., that is bound to the surface, is used to determine the amount of tau, p-tau, or total tau, present in the sample.

In embodiments, the extending step comprises binding the nucleic acid probe to a template oligonucleotide and extending the nucleic acid probe by polymerase chain reaction (PCR), ligase chain reaction (LCR), strand displacement amplification (SDA), self-sustained synthetic reaction (3SR), isothermal amplification (such as, e.g., helicase-dependent amplification or rolling circle amplification (RCA)), or combination thereof. In embodiments, the nucleic acid probe is extended by PCR. In embodiments, the extending step comprises binding the nucleic acid probe of the detection reagent to a template oligonucleotide, ligating the template oligonucleotide to form a circular template oligonucleotide (e.g., by ligation of a linear template oligonucleotide to form a circular oligonucleotide), and extending the nucleic acid probe by RCA.

In embodiments, the sample is contacted with the detection reagent and the capture reagent simultaneously or substantially simultaneously. In embodiments, the sample is contacted with: first, the capture reagent; and second, the detection reagent. In embodiments, the sample is contacted with: first, the detection reagent, and second, the capture reagent. In embodiments, the capture reagent is immobilized on the surface prior to formation of the complex. In embodiments, the complex is formed, then the capture reagent is immobilized to the surface.

In embodiments, the method comprises: contacting the sample with the detection reagent in solution to form a first complex; contacting the first complex with the capture reagent to form a second complex that comprises the capture reagent, tau (e.g., p-tau, including pTau217 and pTau231, or non-phosphorylated tau), and the detection reagent; then, extending the nucleic acid probe as described herein. In embodiments, the method comprises: contacting the sample with the detection reagent in solution to form a first complex comprising tau (e.g., p-tau, including pTau217 and pTau231, or non-phosphorylated tau) and the detection reagent; extending the nucleic acid probe and forming an extended sequence as described herein; then, binding the extended sequence to the surface via the anchoring reagent on the surface and/or binding the first complex to the surface via the capture reagent.

In embodiments, the method comprises contacting the sample with a first detection reagent and a second detection reagent, thereby forming a complex comprising the capture reagent, the tau (e.g., p-tau or t-tau), the first detection reagent, and the second detection reagent. In embodiments, the method comprises detecting the complex, thereby detecting p-tau and/or t-tau. In embodiments, the method further comprises binding the complex to a surface prior to the detecting. In embodiments, the complex is bound to the surface via immobilization of the capture reagent to the surface. In embodiments, the complex is formed on the surface. In embodiments, the complex is formed in solution, then bound to the surface. Formation of the complex and surfaces are further described herein.

In embodiments, the first detection reagent is an antibody, antigen, ligand, receptor, oligonucleotide, hapten, epitope, mimotope, or an aptamer. In embodiments, the first detection reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the first detection reagent comprises at least one heavy or light chain complementarity determining region (CDR) of an antibody. In embodiments, the first detection reagent comprises at least two CDRs from one or more antibodies. In embodiments, the first detection reagent is an antibody or antigen-binding fragment thereof. In embodiments, the first detection reagent binds tau. In embodiments, the first detection reagent is capable of binding to non-phosphorylated tau. In embodiments, the first detection reagent is capable of binding to p-tau. In embodiments, the first detection reagent is capable of binding to both non-phosphorylated tau and p-tau. In embodiments, the first detection reagent is capable of binding p-tau at any amino acid position, site or residue of any one of SEQ ID NOs: 1-9. In embodiments, the first detection reagent is capable of binding p-tau at any phosphorylated amino acid position, site or residue of any one of SEQ ID NO: 1-9. In embodiments, the first detection reagent is capable of binding p-tau that is phosphorylated at T175, T205, T181, T212, S214, T217, cis T231, trans T231, S293, S396, or a combination thereof of SEQ ID NO: 1. In embodiments, the first detection reagent is capable of binding L243. In embodiments, the first detection reagent is capable of binding p-tau that is phosphorylated at T231.

In embodiments, the second detection reagent is an antibody, antigen, ligand, receptor, oligonucleotide, hapten, epitope, mimotope, or an aptamer. In embodiments, the second detection reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the second detection reagent comprises at least one heavy or light chain complementarity determining region (CDR) of an antibody. In embodiments, the second detection reagent comprises at least two CDRs from one or more antibodies. In embodiments, the second detection reagent is an antibody or antigen-binding fragment thereof.

In embodiments where the method detects p-tau, the second detection reagent specifically binds pTau181. In embodiments, the second detection reagent binds pTau181 and does not bind non-phosphorylated tau. In embodiments, the second detection reagent binds pTau181 and does not bind p-tau that is not phosphorylated at amino acid position T181. In embodiments, the method utilizing a second detection reagent that binds p-tau, e.g., pTau181, and that does not bind non-phosphorylated tau has improved specificity as compared to a method that utilizes a second detection reagent that is capable of binding non-phosphorylated tau.

In embodiments where the method detects tau or p-tau, the second detection reagent is capable of binding non-phosphorylated tau and/or p-tau, for example, p-tau that is phosphorylated at any one of amino acid positions or sites: T175, T181, T205, T212, S214, T217, cis T231, trans T231, S293, S396, or a combination thereof of SEQ ID NO: 1. In embodiments, the second detection reagent is capable of binding p-tau at any one of amino acid position, site or residue of SEQ ID NOs: 1-9. In embodiments, the second detection reagent is capable of binding p-tau at any phosphorylated amino acid position, site or residue of SEQ ID NOs: 1-9. In embodiments, the second detection reagent is capable of binding L243.

In embodiments, either the first or the second detection reagent binds pTau181, pTau205, pTau212, pTau217, or pTau231, and does not bind non-phosphorylated tau. In embodiments, the capture reagent binds pTau217, the first detection reagent binds pTau231, and the second detection reagent binds a third phosphorylated tau site, a non-phosphorylated tau site, or the brain-associated tau polypeptide. In embodiments, the capture reagent binds pTau231, the first detection reagent binds pTau217, and the second detection reagent binds a third phosphorylated tau site, a non-phosphorylated tau site, or the brain-associated tau polypeptide. In embodiments, the second detection reagent binds the brain-associated tau polypeptide. In embodiments, the capture reagent binds a phosphorylated tau site, a non-phosphorylated tau site, or the brain-associated tau polypeptide, the first detection reagent binds pTau231, and the second detection reagent binds pTau217. In embodiments, the capture reagent binds a phosphorylated tau site, a non-phosphorylated tau site, or the brain-associated tau polypeptide, the first detection reagent binds pTau217, and the second detection reagent binds pTau231. In embodiments, the capture reagent binds a surface, thereby forming the complex on the surface comprising the capture reagent, the p-tau, the first detection reagent and the second detection reagent, wherein the first detection reagent or second detection reagent comprises a detectable label.

In embodiments, the first detection reagent and/or the second detection reagent comprises a detectable label. In embodiments, the detecting step of the method comprises measuring the amount of the detectable label as described herein. In embodiments where the complex is bound to a surface, the method comprises measuring the amount of detectable label on the surface. In embodiments, the detectable label is capable of being measured by light scattering, optical absorbance, fluorescence, chemiluminescence, electrochemiluminescence (ECL), bioluminescence, phosphorescence, radioactivity, magnetic field, or combinations thereof. In embodiments, the detectable label comprises one or more ECL labels, and the detecting comprises measuring an ECL signal. In embodiments, the amount of measured detectable label, e.g., the amount of measured ECL signal, is used to determine the amount of tau, e.g., p-tau or total tau, is present in the sample. In embodiments, the detectable label comprises a fluorescent label. In embodiments, the detectable label comprises an electrochemiluminescence (ECL) label.

In embodiments, the first detection reagent comprises a first nucleic acid probe, and the second detection reagent comprises a second nucleic acid probe. In embodiments, the detecting step of the method comprises: (i) extending the first and/or second nucleic acid probe to form an extended sequence; and (ii) measuring the amount of the extended sequence. In embodiments, the complex is bound to a surface prior to the detecting, and the surface further comprises an anchoring reagent. In embodiments, the detecting step of the method comprises: (i) extending the first and/or second nucleic acid probe to form an extended sequence comprising an anchoring region that binds to the anchoring reagent; (ii) binding the extended sequence to the anchoring reagent; and (iii) measuring the amount of extended sequence bound to the surface. In embodiments, the amount of measured extended sequence, e.g., that is bound to the surface, is used to determine the amount of tau, e.g., p-tau or total tau, is present in the sample. Methods of extending the first and/or second nucleic acid probe are further described herein. In embodiments, the extending comprises binding the first and/or second nucleic acid probe to a template oligonucleotide and extending the first and/or second nucleic acid probe, e.g., by PCR, LCR, SDA, 3SR, an isothermal amplification method such as helicase-dependent amplification or RCA, or a combination thereof. In embodiments, the extending comprises binding the first and/or second nucleic acid probe to a template oligonucleotide, ligating the template oligonucleotide to form a circular template oligonucleotide (e.g., by ligation of a linear template oligonucleotide to form a circular oligonucleotide), and extending the first and/or second nucleic acid probe, e.g., by RCA. In embodiments, the extending comprises binding the first and second nucleic acid probes to first and second connector oligonucleotides, wherein the first connector oligonucleotide comprises a first connector sequence complementary to a first region of the first nucleic acid probe and a first region of the second nucleic acid probe, and the second connector oligonucleotide comprises a second connector sequence complementary to a second region of the first nucleic acid probe and a second region of the second nucleic acid probe, wherein the first and second regions on each of the first and second nucleic acid probes are non-overlapping; ligating the first and second connector oligonucleotides to form a circular template oligonucleotide; and extending the first and/or second nucleic acid probe, e.g., by RCA.

In embodiments, the sample is contacted with the first and second detection reagents and the capture reagent simultaneously or substantially simultaneously. In embodiments, the sample is contacted with: first, the capture reagent; and second, the first and second detection reagents. In embodiments, the sample is contacted with: first, the first and second detection reagents, and second, the capture reagent. In embodiments, the capture reagent is immobilized on the surface prior to formation of the complex. In embodiments, the complex is formed, then the capture reagent is immobilized to the surface.

In embodiments, the method comprises: contacting the sample with the first and second detection reagents in solution to form a first complex; contacting the first complex with the capture reagent to form a second complex that comprises the capture reagent, tau (e.g., p-tau, including pTau217 and pTau231, or non-phosphorylated tau), and the first and second detection reagents; then, extending the first and/or second nucleic acid probe as described herein. In embodiments, the method comprises: contacting the sample with the first and second detection reagents in solution to form a first complex comprising tau (e.g., pTau, including pTau181, or non-phosphorylated tau) and the first and second detection reagents; extending the first and/or second nucleic acid probe and forming an extended sequence as described herein; then, binding the extended sequence to the surface, e.g., via an anchoring reagent on the surface, and/or binding the first complex to the surface via the capture reagent.

### Anchoring Reagent

The present disclosure includes improved methods that comprise (i) anchoring the detection complex formed between the target analyte and one or more analyte binding reagents used in the assay; and/or (ii) amplifying the signal from labeled detection complexes. Anchoring may be used to stabilize complexes involving low binding affinity interactions and/or high molecular weight label(s) or labeling site(s) (WO2014165061). Signal amplification can be achieved by attaching an extended oligonucleotide to the binding complex that contains multiple labels or detection labeling sites, thereby amplifying the detectable signal for each individual detection complex. In embodiments, the method includes attaching an extended oligonucleotide that includes multiple labels or detection labeling sites to the detection complex, and anchoring the complex to the surface to ensure that the complex is retained on the surface. This assay method can be used to detect extremely low numbers of binding events, even individual analyte-binding reagent complexes. In embodiments, the method is an immunoassay. One of ordinary skill in the art would readily recognize that the advantageous anchoring step described herein is not limited to immunoassays and may be used in other binding assays that utilize other classes of binding reagents.

In embodiments, the extended sequence, formed by extending the nucleic acid probe of the detection reagent or by extending the first and/or second nucleic acid probe of the second detection reagent as described herein, binds to an anchoring reagent on the surface. In embodiments, the anchoring reagent comprises an oligonucleotide, aptamer, aptamer ligand, antibody, antigen, ligand, receptor, hapten, epitope, or a mimotope. In embodiments, the extended sequence comprises an aptamer, and the anchoring reagent comprises a ligand for the aptamer. In embodiments, the extended sequence comprises an oligonucleotide, and the anchoring reagent comprises an oligonucleotide-binding protein that binds the oligonucleotide. In embodiments, the extended sequence comprises a hapten, and the anchoring reagent comprises an antibody specific for the hapten. In embodiments, the extended sequence comprises a receptor, and the anchoring reagent comprises a ligand for the receptor, e.g., a nucleobase conjugated to or modified with the ligand.

In embodiments, the anchoring reagent comprises an anchoring oligonucleotide. In embodiments, the anchoring oligonucleotide is a single stranded oligonucleotide. In embodiments, the extended sequence comprises an anchoring oligonucleotide complement that is complementary to the anchoring oligonucleotide. In embodiments, binding the extended sequence to the anchoring reagent comprises hybridizing the anchoring oligonucleotide complement to the anchoring oligonucleotide.

In embodiments, the anchoring oligonucleotide is a double stranded oligonucleotide. In embodiments, binding the extended sequence to the anchoring reagent comprises forming a triple helix between the anchoring oligonucleotide and the extended sequence. In embodiments, binding the extended sequence to the anchoring reagent comprises denaturing the anchoring oligonucleotide to provide a single stranded oligonucleotide region that binds the extended sequence. In embodiments, binding the extended sequence to the anchoring reagent comprises subjecting the anchoring oligonucleotide to a helicase and/or nuclease to provide an oligonucleotide region that binds the extended sequence.

In embodiments, the amount of extended sequence bound to the surface is measured. In embodiments, the amount of extended sequence is measured without binding the extended sequence to the surface, e.g., via the anchoring reagent as described herein. In embodiments, the measuring comprises contacting the extended sequence with a labeled probe that binds to the extended sequence and that comprises a detectable label. In embodiments, the labeled probe comprises a detection oligonucleotide and a detectable label, and the extended sequence comprises a detection oligonucleotide complement that is complementary to the detection oligonucleotide. In embodiments, the labeled probe comprises one or more detectable labels. Detectable labels are further described herein. In embodiments, the detectable label is capable of being measured by a measurement of light scattering, optical absorbance, fluorescence, chemiluminescence, electrochemiluminescence (ECL), bioluminescence, phosphorescence, radioactivity, magnetic field, or combinations thereof. In embodiments, the detectable label comprises one or more ECL labels, and the measuring comprises measuring an ECL signal. In embodiments, the amount of measured ECL signal is used to determine the amount of tau, e.g., p-tau or total tau, in the sample. In embodiments, the detectable label comprises a fluorescent label. In embodiments, the detectable label comprises an electrochemiluminescence (ECL) label.

In embodiments, the extended sequence comprises a labeled base, wherein the labeled base comprises a detectable label, and the measuring comprises measuring the detectable label. In embodiments, the extended sequence comprises a modified base, and the measuring comprises contacting the extended sequence with a detectable moiety that binds to the modified base and that comprises a detectable label. In embodiments, the modified base comprises an aptamer, aptamer ligand, antibody, antigen, ligand, receptor, hapten, epitope, or a mimotope, and the detectable moiety comprises a binding partner of the modified base. In embodiments, the modified base comprises avidin, streptavidin, and/or an antibody specific for biotin, and the detectable moiety comprises biotin and a detectable label. In embodiments, the modified base comprises biotin, and the detectable moiety comprises avidin, streptavidin, and/or an antibody specific for biotin and a detectable label. In embodiments, the detectable moiety comprises one or more detectable labels. Detectable labels are further described herein. In embodiments, the detectable label comprises one or more ECL labels, and the measuring comprises measuring an ECL signal. In embodiments, the amount of measured ECL signal is used to determine the amount of tau, e.g., p-tau or tau, in the sample.

### Measuring Step

In embodiments, the measuring step is performed by methods known in the art, for example, immune-based methods such as Enzyme-Linked ImmunoSorbent Assay (ELISA), western blotting, immunoprecipitation (IP), flow cytometry, immunohistochemistry (IHC), immunocytochemistry (ICC), or Enzyme-linked Immunospot (ELISPOT).

In embodiments in which an extended sequence is formed, the measuring step of the method comprises measuring the amount of extended sequence. In embodiments, the measuring comprises contacting the extended sequence with a labeled probe that binds to the extended sequence. In embodiments, the extended sequence comprises a detection sequence complement that is complementary to a detection oligonucleotide, and measuring the amount of extended sequence comprises contacting the extended sequence with a labeled probe comprising the detection oligonucleotide and a detectable label. In embodiments, the extended sequence comprises a modified base, and measuring the amount of extended sequence comprises contacting the extended sequence with a detectable moiety capable of binding to the modified base. In embodiments, the modified base comprises an aptamer, aptamer ligand, antibody, antigen, ligand, receptor, hapten, epitope, or a mimotope, and the detectable moiety comprises a binding partner of the modified base and a detectable label. In embodiments, the modified base comprises streptavidin, and the detectable moiety comprises biotin and a detectable label. In embodiments, the modified base comprises avidin, and the detectable moiety comprises biotin and a detectable label. In embodiments, the modified base comprise biotin, and the detectable moiety comprises avidin and a detectable label.

In embodiments comprising a labeled probe or a detectable moiety that comprises a detectable label, the detectable label is measured by a measurement of light scattering, optical absorbance, fluorescence, chemiluminescence, electrochemiluminescence, bioluminescence, phosphorescence, radioactivity, magnetic field, or combinations thereof. In embodiments, the detectable label is an electrochemiluminescent (ECL) label, and measuring the extended sequence comprises measuring an ECL signal. In embodiments, the labeled probe or detectable moiety comprises multiple detectable labels, e.g., multiple ECL labels. In embodiments, the detectable label comprises ruthenium. In embodiments, the amount of measured ECL signal is used to determine the amount of p-tau in the biological sample. In embodiments, the amount of measured ECL signal is used to determine the amount of t-tau in the biological sample. In embodiments, the detectable label comprises a fluorescent label. In embodiments, the detectable label comprises an electrochemiluminescence (ECL) label.

In embodiments where the detection reagent comprises a detectable label, the measuring step of the method comprises measuring the presence and/or amount of the detectable label. In embodiments, the detectable label is measured by light scattering, optical absorbance, fluorescence, chemiluminescence, electrochemiluminescence (ECL), bioluminescence, phosphorescence, radioactivity, magnetic field, or combinations thereof. In embodiments, the detectable label comprises an ECL label, and measuring the amount of detectable label comprises measuring an ECL signal. In embodiments, the detectable label comprises multiple ECL labels. In embodiments, the detectable label comprises ruthenium. In embodiments, the amount of measured ECL signal is used to determine the amount of p-tau in the biological sample. In embodiments, the amount of measured ECL signal is used to determine the amount of t-tau in the biological sample. In embodiments, the detectable label comprises a fluorescent label. In embodiments, the detectable label comprises an electrochemiluminescence (ECL) label.

### Additional Methods

In some embodiments, the method of detecting, quantifying, or both, p-tau in a biological sample as described herein comprises: contacting the biological sample with: (i) a capture reagent that binds to a phosphorylated tau site, or a non-phosphorylated tau site; (ii) a first detection reagent that binds to a phosphorylated tau site, or a non-phosphorylated tau site; and (iii) a second detection reagent that binds to a phosphorylated tau site, or a non-phosphorylated tau site. In some embodiments, the first detection reagent is conjugated to a first nucleic acid probe (NAP1) and the second detection reagent is conjugated to a second nucleic acid probe (NAP2). In embodiments, the method comprises forming a complex comprising the capture reagent, the p-tau, the first detection reagent and the second detection reagent. In embodiments, the method comprises producing an extended oligonucleotide by extending the NAP1 and/or the NAP2.

In embodiments, the extended oligonucleotide is produced by a method comprising: (i) contacting a first connector oligonucleotide (CON1) and a second connector oligonucleotide (CON2) with the NAP1 and the NAP2, wherein the NAP1 binds to a 5′ end of the CON1 and to a 3′ end of the CON2 and the NP2 binds to a 3′ end of the CON1 and to a 5′ end of the CON2; (ii) ligating the 5′ end of the CON1 with the 3′ end of CON2, and ligating the 3′ end of the CON1 with the 5′ end of the CON2, to form a circular template oligonucleotide, and (iii) extending the NAP1 and/or the NAP2 to produce the extended oligonucleotide.

In some embodiments, the extended oligonucleotide is produced by a method comprising: (i) contacting a single connector oligonucleotide with the NAP1 and the NAP2, wherein the NAP1 binds to 5′ and 3′ ends of the single connector oligonucleotide and the NAP2 binds to the single connector oligonucleotide at a different site than the NP1; (ii) ligating the 5′ and 3′ ends of the single connector oligonucleotide to form a circular template oligonucleotide; and (iii) extending the NAP1 and/or the NAP2 to produce the extended oligonucleotide.

In some embodiments, the circular template oligonucleotide comprises about 40% to about 80% thymine (T). In some embodiments, the circular template oligonucleotide is about 30 to about 80 bases in length. In some embodiments, the circular template oligonucleotide comprises a detection sequence, and one or both of the NAP1 and the NAP2 binds to at least 80% of the detection sequence, the extended oligonucleotide comprises a detection sequence complement, and the method further comprises binding to the extended oligonucleotide to a detection oligonucleotide that binds to the detection sequence complement. In some embodiments, the circular template oligonucleotide comprises at least three detection sequences, the extended oligonucleotide comprises at least three detection sequence complements, and the method further comprises binding to the extended oligonucleotide to one or more detection oligonucleotides, wherein each detection oligonucleotide binds to one of the at least three detection sequence complements. In some embodiments, the CON1 and the CON2 are each about 15 to about 40 bases in length and do not differ by more than 5, 6, or 7 bases in length. In some embodiments, the CON1 comprises an anchoring sequence and the CON2 comprises a detection sequence, and the method further comprises binding to the extended oligonucleotide to (I) a detection oligonucleotide that binds to a detection sequence complement; and (II) an anchoring reagent that binds to an anchoring sequence complement, wherein the anchoring reagent is on a surface or capable of being bound to a surface. In embodiments, the method comprises detecting the extended oligonucleotide, thereby detecting the p-tau; or measuring an amount of the extended oligonucleotide, thereby quantifying an amount of the p-tau.

In some embodiments, the method of detecting, quantifying, or both, p-tau in a biological sample as described herein comprises: contacting the biological sample with: (i) a capture reagent that binds to a phosphorylated tau site, or a non-phosphorylated tau site; and (ii) a detection reagent that binds to a phosphorylated tau site, or a non-phosphorylated tau site. In some embodiments, the detection reagent is conjugated to a first nucleic acid probe (NAP1). In embodiments, the method comprises forming a complex comprising the capture reagent, the p-tau, and the detection reagent. In embodiments, the method comprises producing an extended oligonucleotide by extending the NAP1.

In embodiments, the extended oligonucleotide is produced by a method comprising: (i) (i) contacting a single connector oligonucleotide with the NAP1, wherein the NAP1 binds to 5′ and 3′ ends of the single connector oligonucleotide; (ii) ligating the 5′ and 3′ ends of the single connector oligonucleotide to form a circular template oligonucleotide; and (iii) extending the NAP1 to produce the extended oligonucleotide

In embodiments, the detection reagent is further conjugated to a second nucleic acid probe (NAP2), and the extended oligonucleotide is produced by a method comprising: (i) contacting a first connector oligonucleotide (CON1) and a second connector oligonucleotide (CON2) with the NAP1 and the NAP2, wherein the NAP1 binds to a 5′ end of the CON1 and to a 3′ end of the CON2 and the NP2 binds to a 3′ end of the CON1 and to a 5′ end of the CON2; (ii) ligating the 5′ end of the CON1 with the 3′ end of CON2, and ligating the 3′ end of the CON1 with the 5′ end of the CON2, to form a circular template oligonucleotide, and (iii) extending the NAP1 and/or the NAP2 to produce the extended oligonucleotide.

In some embodiments, the circular template oligonucleotide about 40% to about 80% thymine (T). In some embodiments, the circular template oligonucleotide is about 30 to about 80 bases in length and comprises about 30% to about 80% T. In some embodiments, the circular template oligonucleotide comprises a detection sequence, the NP1 binds to at least 80% of the detection sequence, the extended oligonucleotide comprises a detection sequence complement, and the method further comprises binding the extended oligonucleotide to a detection oligonucleotide that binds to the detection sequence complement. In some embodiments, the circular template oligonucleotide comprises at least three detection sequences, the extended oligonucleotide comprises at least three detection sequence complements, and the method further comprises binding the extended oligonucleotide to one or more detection oligonucleotides, wherein each detection oligonucleotide binds to a one of the at least three detection sequence complements. In embodiments, the method comprises detecting the extended oligonucleotide, thereby detecting the p-tau; or measuring an amount of the extended oligonucleotide, thereby quantifying an amount of the p-tau.

In some embodiments, the circular template oligonucleotide comprises (a) about 60% to about 73% T, about 14% to about 24% C, about 15% to about 22% G, and about 0% to about 4% A; (b) about 62% to about 69% T, about 15% to about 18% C, about 16% to about 20% G, and about 1% to about 3% A; or (c) about 63.3% T, about 16.7% C, about 18.3% G, and about 1.7% A. In some aspects, the circular template oligonucleotide is (a) about 40 to about 60 bases in length, (b) 48 to about 60 bases in length, or (c) 60 bases in length. In embodiments, the circular template oligonucleotide comprises repeated GTT motifs.

In embodiments, the method further comprises a second capture reagent. In embodiments, the method further comprises a third detection reagent. In embodiments, at least one of the capture reagent, the first detection reagent, and the second detection reagent binds to an amino acid position or site of any one of SEQ ID NO: 1-9. In embodiments, at least one of the capture reagent, the first detection reagent, and the second detection reagent binds to an amino acid position or site of SEQ ID NO: 1. In embodiments, at least one of the capture reagent, the first detection reagent, and the second detection reagent binds an amino acid position or site of SEQ ID NO: 2. In embodiments, at least one of the capture reagent, the first detection reagent, and the second detection reagent binds an amino acid position or site of SEQ ID NO: 3. In embodiments, at least one of the capture reagent, the first detection reagent, and the second detection reagent binds to an amino acid position or site of SEQ ID NO: 4. In embodiments, at least one of the capture reagent, the first detection reagent, and the second detection reagent binds to an amino acid position or site of SEQ ID NO:5. In embodiments, at least one of the capture reagent, the first detection reagent, and the second detection reagent binds to an amino acid position or site of SEQ ID NO: 6. In embodiments, at least one of the capture reagent, the first detection reagent, and the second detection reagent binds to an amino acid position or site of SEQ ID NO: 7. In embodiments, at least one of the capture reagent, the first detection reagent, and the second detection reagent binds to an amino acid position or site of SEQ ID NO: 8. In embodiments, at least one of the capture reagent, the first detection reagent, and the second detection reagent binds to an amino acid position or site of SEQ ID NO: 9. In embodiments, the first capture reagent, the second capture reagent, the first detection reagent, the second detection reagent, and/or the third detection reagent bind to a phosphorylated or non-phosphorylated tau site according to any one of the combinations shown in Table 2.

### ImmunoSequencing

In embodiments where a complex comprising a plurality of binding reagents and tau is formed, each binding reagent is linked to an oligonucleotide. In embodiments, the method further comprises ligating and/or extending the oligonucleotide of each of the binding reagents to generate an output oligonucleotide. In embodiments, generating the output oligonucleotide comprises polymerase chain reaction (PCR), ligase chain reaction (LCR), strand displacement amplification (SDA), self-sustained synthetic reaction (3SR), isothermal amplification, or combination thereof. In embodiments, the detecting and/or measuring the complex comprises contacting the output oligonucleotide with a detectable label and measuring the amount of detectable label bound to the surface.

In embodiments, the present disclosure provides methods of determining and measuring target epitopes of tau, comprising: contacting the tau with a plurality of binding reagents, wherein each binding reagent comprises a detection sequence comprising a barcode oligonucleotide sequence, wherein if at least three binding reagents bind to three tau epitopes, an output oligonucleotide is generated that comprises the barcode oligonucleotide sequences of each of the three or more binding reagents; and sequencing the output oligonucleotide to identify the barcode oligonucleotide sequences, thereby determining the at least three tau epitopes, as described, e.g., in WO2019/222708; WO2020/086751; WO2022/051481; WO2023/212315; U.S. Pat. No. 9,499,858; US2021/0389304; US2021/0382043; US2023/0349920; US2025/0035615; and US2024/0409983.

In embodiments, each binding reagent comprises an oligonucleotide comprising a unique barcode sequence. In embodiments, at least two binding reagents comprise an oligonucleotide. In embodiments, the first binding reagent and the second binding reagent comprise an oligonucleotide. In embodiments, the first binding reagent, the second binding reagent, and the third binding reagent comprise an oligonucleotide. In embodiments, the first binding reagent, the second binding reagent, the third binding reagent, and the fourth binding comprise an oligonucleotide. In embodiments, the method comprises an anchoring reagent, wherein the anchoring reagent comprises an oligonucleotide. In embodiments, the detecting and/or measuring the complex of the method disclosed herein comprises amplifying the output oligonucleotide and adding one or more sequencing primer sites to the output oligonucleotide. In embodiments, the method further comprises sequencing the amplified output oligonucleotide to identify the barcode sequences of the binding reagents.

In embodiments the method comprises a first capture reagent, a first detection reagent, and second detection reagent. In embodiments, at least two of the first capture reagent, the first detection reagent, and the second detection reagent comprise an oligonucleotide. In embodiments, the first capture reagent, the first detection reagent, and the second detection reagent each comprises an oligonucleotide. In embodiments, the method further comprises a third detection reagent and/or second capture reagent. In embodiments, the third detection reagent comprises an oligonucleotide. In embodiments, the second capture reagent comprises an oligonucleotide.

In embodiments, the plurality of binding reagents comprises: a first binding reagent comprising a first detection sequence that comprises a first hybridization sequence, and a first amplification primer site; a second binding reagent comprising a second detection sequence that comprises a second hybridization sequence, and a third hybridization sequence; and a third binding reagent comprising a third detection sequence that comprises a fourth hybridization sequence, and a second amplification primer site, wherein the first hybridization sequence and the second hybridization sequence are complementary; wherein the third hybridization sequence and the fourth hybridization sequence complementary; and wherein generating the single output oligonucleotide comprises ligating, extending, or both, the hybridized first detection sequence, second detection sequence, and third detection sequence. In embodiments, the single output oligonucleotide is amplified by quantitative polymerase chain reaction (qPCR) and sequenced by next-generation sequencing (NGS).

### Immobilized and Solution Formats

In embodiments, the capture reagent is immobilized to the surface. In embodiments, the capture reagent is directly immobilized on the surface. In embodiments, the capture reagent is indirectly immobilized on the surface via secondary binding reagents, e.g., a targeting agent. In embodiments, the capture reagent is linked to a targeting agent complement that binds to a targeting agent immobilized on the surface. In embodiments, the targeting agent complement directly binds to the targeting agent. In embodiments, the targeting agent and targeting agent complement comprise complementary oligonucleotides, a receptor-ligand pair, an antigen-antibody pair, a hapten-antibody pair, an epitope-antibody pair, a mimotope-antibody pair, an aptamer-target molecule pair, hybridization partners, or an intercalator-target molecule pair. In embodiments, the targeting agent and targeting agent complement are cross-reactive moieties, e.g., thiol and maleimide or iodoacetamide; aldehyde and hydrazide; or azide and alkyne or cycloalkyne. In embodiments, the targeting agent is biotin, and the targeting agent complement is avidin or streptavidin. In embodiments, the targeting agent complement binds to the targeting agent via a targeting bridge agent, which is a binding partner of both the targeting agent and the targeting agent complement. In embodiments, the targeting bridge agent comprises multiple binding sites. In embodiments, the targeting bridge agent is streptavidin or avidin, and the targeting agent and targeting agent complement are each biotin.

In embodiments comprising first and second detection reagents, the method comprises binding the analyte, e.g., tau or p-tau, to the first and second detection reagents in solution to form a first complex, contacting the first complex to the capture reagent on the surface to form a second complex comprising the capture reagent, the analyte, e.g., tau or p-tau, and the first and second detection reagents, then extending the first and/or second nucleic acid probe as described herein. In embodiments, the method comprises binding the analyte, e.g., tau or p-tau to the first and second detection reagents in solution to form a first complex, extending the first and/or second nucleic acid probe as described herein to form an extended sequence, then binding the extended sequence to the surface via the capture reagent and/or the anchoring reagent.

In embodiments comprising a detection reagent that comprises a nucleic acid probe, the method comprises binding the analyte, e.g., tau or p-tau to the detection reagent in solution to form a first complex, contacting the first complex to the reagent on the surface to form a second complex comprising the capture reagent, the analyte, e.g., tau or p-tau, and the detection reagent, then extending the nucleic acid probe as described herein. In embodiments, the method comprises binding the analyte, e.g., tau or p-tau to the detection reagent in solution to form a first complex, extending the nucleic acid probe as described herein to form an extended sequence, then binding the extended sequence to the surface via the capture reagent and/or the anchoring reagent.

In embodiments comprising a detection reagent that comprises a detectable label, the method comprises binding the analyte, e.g., tau or p-tau to the detection reagent in solution to form a first complex, contacting the first complex to the capture reagent on the surface to form a second complex comprising the capture reagent, the analyte, e.g., tau or p-tau, and the detection reagent, then measuring the amount of detectable label as described herein.

### Surface

In embodiments, the surface comprises a particle. In embodiments, the surface comprises a well of multi-well plate. In embodiments, the surface comprises a plurality of distinct binding domains and the one or more binding reagent immobilized to the surface are located on the same binding domain on the surface. In embodiments, the surface comprises a plurality of distinct binding domains and the one or more binding reagent immobilized to the surface are located at distinct binding domains on the surface. In embodiments, the surface comprises a plurality of distinct binding domains, and the capture reagent is located on a distinct binding domain on the surface. In embodiments, the surface comprises a plurality of distinct binding domains. In embodiments where the surface further comprises an anchoring reagent, the capture reagent and anchoring reagent are located on the same binding domain on the surface. In embodiments where the surface further comprises an anchoring reagent, the capture reagent and anchoring reagent are located on two or more distinct binding domains on the surface.

In embodiments, the capture reagent is immobilized on the surface, e.g., prior to or during the contacting step, or prior to or during the detecting step of the method as described herein. In embodiments, the capture reagent is directly immobilized on the surface, e.g., via a covalent linkage between the capture reagent and the surface. In embodiments, the capture reagent is indirectly immobilized on the surface via a secondary binding reagents, e.g., a targeting agent. In embodiments, the surface comprises a targeting agent, and the capture reagent is linked to a targeting agent complement that is capable of binding to a targeting agent. In embodiments, the targeting agent complement directly binds to the targeting agent. In embodiments, the targeting agent and targeting agent complement comprise complementary oligonucleotides. In embodiments, the targeting agent and targeting agent complement comprise a binding pair selected from a receptor-ligand pair, an antigen-antibody pair, a hapten-antibody pair, an epitope-antibody pair, a mimotope-antibody pair, an aptamer-target molecule pair, hybridization partners, or an intercalator-target molecule pair. In embodiments, the targeting agent and targeting agent complement are cross-reactive moieties, e.g., thiol and maleimide or iodoacetamide; aldehyde and hydrazide; or azide and alkyne or cycloalkyne. In embodiments, the targeting agent is biotin, and the targeting agent complement is avidin, streptavidin, or an antibody specific for biotin.

In embodiments, the detection reagent comprises a nucleic acid probe or the first and/or second detection reagent comprises a first and/or second nucleic acid probe, and the surface comprises (i) the capture reagent as described herein, and (ii) an anchoring reagent. In embodiments, the anchoring reagent is directly immobilized on the surface, e.g., via a covalent linkage between the anchoring reagent and the surface. In embodiments, the anchoring reagent indirectly immobilized on the surface via a secondary binding reagents, e.g., a targeting agent as described herein. In embodiments, the targeting agent and targeting agent complement for the anchoring reagent is selected such that the targeting agent and targeting agent complement associated with the anchoring reagent are substantially non-cross-reactive with the targeting agent and targeting agent complement associated with the capture reagent. In embodiments, the targeting agent and targeting complement associated with the anchoring reagent are cross-reactive with the targeting agent and targeting agent complement associated with the capture reagent.

In embodiments, the targeting agent complement binds to the targeting agent via a targeting bridge agent, which is a binding partner of both the targeting agent and the targeting agent complement. In embodiments, the targeting bridge agent comprises multiple binding sites. In embodiments, the targeting bridge agent is streptavidin or avidin, and the targeting agent and targeting agent complement are each biotin.

The methods of the invention may be applied to singleplex formats or multiplex formats, wherein multiple assay measurements are performed on a single sample. Multiplex measurements that can be used with the invention include, but are not limited to, multiplex measurements i) that involve the use of multiple sensors; ii) that use discrete assay domains on a surface (e.g., an array) that are distinguishable based on location on the surface; iii) that involve the use of reagents coated on particles that are distinguishable based on a particle property such as size, shape, color, etc.; iv) that produce assay signals that are distinguishable based on optical properties (e.g., absorbance or emission spectrum); or v) that are based on temporal properties of assay signal (e.g., time, frequency or phase of a signal).

In a specific embodiment, the methods of the present disclosure are used in a multiplexed format by binding a plurality of different analytes to a plurality of capture reagents for those analytes, the captured analytes being immobilized on coded bead, such that the coding identifies the capture reagent (and analyte target) for a specific bead. The method may further comprise counting the number of beads that have a captured analyte (e.g., using the detection approaches described herein). In embodiments, the multiplexed measurement of analytes on a surface comprising a plurality of binding domains using electrochemiluminescence is used in the Meso Scale Discovery (MSD), MULTI-ARRAY®, MULTI-SPOT®, QuickPlex®, and SECTOR® lines of products (see, e.g., U.S. Pat. Nos. 7,842,246 and 6,977,722) and the MSD website.

### Sample

In embodiments, the sample is a biological sample. In embodiments, the sample comprises a mammalian fluid, secretion, or excretion. In embodiments, the biological sample is a purified mammalian fluid, secretion, or excretion. In embodiments, the mammalian fluid, secretion, or excretion is whole blood, plasma, serum, sputum, lachrymal fluid, lymphatic fluid, synovial fluid, pleural effusion, urine, sweat, cerebrospinal fluid, ascites, milk, stool, bronchial lavage, saliva, amniotic fluid, nasal secretions, vaginal secretions, a surface biopsy, sperm, semen/seminal fluid, wound secretions and excretions, or an extraction or purification therefrom, or dilution thereof. Further exemplary biological samples include but are not limited to physiological samples, samples containing suspensions of cells such as mucosal swabs, tissue aspirates, tissue homogenates, cell cultures, and cell culture supernatants. In embodiments, the biological sample is whole blood, serum, plasma, cerebrospinal fluid, urine, saliva, or an extraction or purification therefrom, or dilution thereof. In embodiments, the biological sample is serum or plasma. In embodiments, the plasma is in EDTA, heparin, or citrate. In embodiments, the biological sample is a mammalian fluid, secretion, or excretion that is known to contain a high level of tau or p-tau. In embodiments, the biological sample is a mammalian fluid, secretion, or excretion that is known to contain a low level of tau or p-tau. In embodiments, the biological sample containing the high or low levels of tau or p-tau is a control for the methods described herein.

In embodiments, the sample is obtained from a subject, e.g., a human subject. In embodiments, the sample comprises plasma (e.g., in EDTA, heparin, citrate, or combination thereof) from a human subject. In embodiments, the sample comprises serum from a human subject.

In embodiments, the sample is obtained from a subject suspected of having, or diagnosed with a tauopathy, e.g., a disease associated with tau disfunction, such as abnormal aggregation of tau protein. In embodiments, the tauopathy is AD, Progressive Supranuclear Palsy (PSP), Corticobasal Degeneration (CBD), Pick's Disease (FTLD-tau subtype), Globular Glial Tauopathy (GGT), Argyrophilic Grain Disease (AGD), Primary Age-Related Tauopathy (PART), Aging-related tau astrogliopathy (ARTAG), Neurofibrillary Tangle-Predominant Dementia (NFTPD).

In embodiments, the sample is obtained from a cognitively normal subject. As described herein, a “cognitively normal subject” is a subject who has not been diagnosed with cognitive impairment, who does not exhibit any cognitive impairment symptoms, who does not have any subjective cognitive complaints (SCC) as described herein, and/or who does not have any diagnoses that puts the individual at risk of cognitive decline in the future.

In embodiments, the sample is obtained from a subject who has been diagnosed with mild or severe cognitive impairment, e.g., as a result of AD. In embodiments, the sample is obtained from a subject who is at increased risk for AD, e.g., due to factors such as brain injury, family history, genetics, and the like.

As described herein, an individual with Alzheimer's Disease (AD) refers to a person who has been diagnosed with AD using a test known to one of skill in the art, for example, clinical presentation, a biomarker test, and/or a brain scan.

As described herein, an individual with “mild cognitive impairment” or “MCI” means that the individual has a slight but noticeable decline in mental abilities, e.g., memory and thinking skills, as compared with others of the same age. The minor decline in abilities is noticeable by the individual experiencing them or by others who interact with the individual, but the changes are not severe enough to interfere with normal daily life and activities.

As described herein, “severe cognitive impairment” (used interchangeably herein with “dementia”) refers to a decline in mental function that is severe enough to interfere with daily living.

As described herein, “subjective cognitive complaints” (SCC) refer to memory and related cognitive concerns expressed by individuals with or without objective evidence of cognitive impairment. An individual with SCC may be diagnosed with MCI, severe cognitive impairment, and/or AD. See, e.g., Mitchell, Age Ageing 37(5):497-499 (2008).

Methods of identifying individuals as cognitively normal or cognitively impaired, e.g., with MCI or dementia, are known to one of ordinary skill in the art, e.g., using neurological examinations, mental status testing, neuropsychological testing, computerized testing, genetic testing, brain imaging, or combination thereof. See, e.g., Neugroschl et al., Mt Sinai J Med. 78(4): 596-612 (2011). For example, the CDR® Dementia Staging Instrument, developed at the Charles F. and Joanne Knight Alzheimer Disease Research Center (Knight ADRC), is a 5-point scale used to characterize six domains of cognitive and functional performance applicable to Alzheimer disease and related dementias: Memory, Orientation, Judgment & Problem Solving, Community Affairs, Home & Hobbies, and Personal Care (knightadre.wustl.edu/cdr/cdr.htm). Other exemplary assessments for dementia include, e.g., the Mini-Mental State Exam (MMSE), the Mini-Cog test, and the Montreal Cognitive Assessment (MoCA) (mocatest.org). Classification of cognitively normal and cognitively impaired individuals is further described, e.g., in Dixon et al., J Clin Exp Neuropsychol 36(4):418-430 (2014). MCI, dementia, and AD are further described, e.g., in Peterson et al., Arch Neurol 56:303-308 (1999).

In embodiments, cognitively normal individuals have plasma p-tau levels of about 0.7 to about 1.9 pg/mL, or about 0.75 to about 1.8 pg/mL, or about 0.8 to about 1.7 pg/mL, or about 0.85 to about 16 μg/mL. In embodiments, cognitively normal individuals have a mean plasma p-tau level of about 1.3±0.6 pg/mL. In embodiments, cognitively normal individuals have plasma total tau levels of about 6 to about 13 μg/mL, or about 7.5 to about 12 μg/mL, or about 7 to about 11.5 pg/mL. In embodiments, cognitively normal individuals have a mean plasma total tau level of about 9±3 μg/mL. In embodiments, individuals with MCI who remain stable have plasma p-tau levels of about 0.9 to about 2.2 pg/mL, or about 0.95 to about 2.1 pg/mL, or about 1 to about 2 μg/mL. In embodiments, individuals with MCI who remain stable have plasma total tau levels of about 6 to about 14 μg/mL, or about 7 to about 13.5 pg/mL, or about 8 to about 13 μg/mL. In embodiments, individuals with MCI who decline have plasma p-tau levels of about 1.8 to about 4 μg/mL, or about 1.85 to about 3.9 pg/mL, or about 1.9 to about 3.8 pg/mL. In embodiments, individuals with MCI who remain stable have plasma total tau levels of about 8 to about 16 μg/mL, or about 8.5 to about 15.5 pg/mL, or about 9 to about 15 μg/mL. In embodiments, individuals with AD have plasma p-tau levels of about 1.5 to about 3.9 pg/mL, or about 1.7 to about 3.7 pg/mL, or about 1.9 to about 3.5 pg/mL. In embodiments, individuals with AD have a mean plasma p-tau level of about 2.7±1.2 pg/mL. In embodiments, individuals with AD have plasma total tau levels of about 8 to about 20 μg/mL, or about 8.5 to about 19 μg/mL, or about 9 to about 18 μg/mL. In embodiments, individuals with AD have a mean plasma total tau level of about 14±6 μg/mL. In embodiments, the p-tau comprises pTau217 and pTau231.

In embodiments, the sample is obtained from the subject within about 10 minutes to about 50 years, about 20 minutes to about 30 years, about 30 minutes to about 10 years, about 40 minutes to about 5 years, about 50 minutes to about 1 year, about 1 hour to about 6 months, about 6 hours to about 1 month, about 12 hours to about 2 weeks, about 1 day to about 7 days, about 2 days to about 6 days, or about 3 days to about 4 days after being diagnosed with MCI, dementia, or AD. In embodiments, the biological sample is obtained from the subject about 1 minute, about 10 minutes, about 30 minutes, about 1 hour, about 3 hours, about 6 hours, about 12 hours, about 24 hours, about 2 days, about 3 days, about 4 days, about 5 days, about 6 days, about 1 week, about 2 weeks, about 1 month, about 3 months, about 6 months, about 1 year, about 2 years, about 3 years, about 5 years, about 10 years, about 20 years, about 30 years, or more than 30 years after being diagnosed with MCI, dementia, or AD. In embodiments, the sample is obtained from the subject once a week, 2 weeks, 1 month, 2 months, 3 months, 6 months, 9 months, 1 year, 2 years, 3 years, 5 years, or more, over the course of 1 year, 2 years, 3 years, 4 years, 5 years, 6 years, 7 years, 8 years, 9 years, 10 years, or more than 10 years.

Samples may be obtained from a single source described herein, or may contain a mixture from two or more sources, e.g., pooled from one or more subjects who may have been, for example, diagnosed with, suspected to have, or at risk of developing dementia or AD. In embodiments, the present disclosure provides a method of determining the relative amount or percentage of tau phosphorylated or non-phosphorylated at a specific site within one population (i.e., % phosphorylated site/total detected site or % phosphorylated site/non-phosphorylated site). In embodiments, the present disclosure provides a method of determining the relative amount or percentage of tau phosphorylated or non-phosphorylated at a specific site between a first and second population of tau in a biological sample. In embodiments, the biological sample comprises: a first population of tau comprising a first epitope comprising the phosphorylated first site; and a second population comprising the first epitope comprising the non-phosphorylated first site. In embodiments, the method comprises, e.g., determining a risk of developing disease, determining disease condition, and/or determining disease progression, based on the ratio of phosphorylated to non-phosphorylated first site in the biological sample or other ways of partitioning individuals, e.g., for research purposes.

Alzheimer's Disease, Cognitive Impairment and/or Decline

As discussed herein, diagnosis of early stage Alzheimer's disease (AD) is valuable for early and more effective treatment and for enrolling patients into clinical trials. Early stage diagnosis is also important for identifying individuals for whom treatment would likely provide the greatest benefit. In embodiments, the present disclosure provides a method of measuring the levels of p-tau. In embodiments, the present disclosure provides methods of measuring the levels of total tau, i.e., non-phosphorylated tau and p-tau, wherein the p-tau is phosphorylated at any of its serine (Ser) or threonine (Thr) residues. In embodiments, the p-tau comprises pTau217 and pTau231.

In embodiments, the p-tau comprises SEQ ID NO: 1-9 or a fragment or variant thereof, phosphorylated at one or more sites. In embodiments, the p-tau comprises any one of the eighty-five serine (S), threonine (T), and tyrosine (Y) phosphorylated tau sites. In embodiments, the p-tau comprises any phosphorylated site selected from: T175, T181, T205, T212, S214, T217, cis or trans T231, S293, or S396, of SEQ ID NO: 1. In embodiments, the p-tau comprises t-tau. In embodiments, the p-tau comprises a commonly phosphorylated site. In embodiments, the p-tau comprises a rarely phosphorylated site. In embodiments, the p-tau comprises a never phosphorylated site. In embodiments, the p-tau comprises any one of the other tau sites that are not one of the eighty-five serine (S), threonine (T), and tyrosine (Y) tau phosphorylated sites.

The present disclosure provides a method of detecting a brain injury, brain injury severity, or a neurodegenerative disease in a subject, comprising obtaining measured levels of p-tau in a sample from the subject as described herein. In embodiments, the sample is whole blood, cerebrospinal fluid (CSF), serum, plasma, or a combination thereof.

In embodiments, the present disclosure provides a method of detecting brain injury or brain injury severity. In embodiments, the brain injury is concussive injury, subconcussive injury, acute concussive injury, impact head injury, acceleration or deceleration head trauma, closed-skull neurotrauma, traumatic brain injury, stroke, seizure, status epilepticus, chronic traumatic encephalopathy (CTE), or a combination thereof.

In embodiments, the present disclosure provides a method of detecting a neurodegenerative disease. In embodiments, the neurodegenerative disease is Alzheimer's disease, amyotrophic lateral sclerosis, Friedreich ataxia, Huntington's disease, Lewy body disease, Parkinson's disease, spinal muscular atrophy, Creutzfeldt-Jakob disease, Neuronal Synuclein Disease (NSD), motor neuron disease, or any combination thereof. In embodiments, the neurodegenerative disease is Alzheimer's disease.

In embodiments, the present disclosure provides a method of determining eligibility of a subject to participate in a clinical trial of a therapeutic drug for preventing or delaying Alzheimer's disease, the method comprising: (a) obtaining a measurement of phosphorylated tau levels of the subject as described herein; and (b) determining the eligibility of the subject for the clinical trial based on the measurement of phosphorylated tau levels; wherein the subject has been diagnosed with dementia or mild cognitive impairment, or wherein the subject has subjective cognitive complaints, or wherein the subject does not have any cognitive impairment.

In embodiments, the present disclosure provides a method of conducting a clinical trial of a therapeutic drug or intervention for Alzheimer's disease, the method comprising: (a) obtaining a measurement of phosphorylated tau levels of a subject as described herein; (b) determining eligibility of the subject for the clinical trial based on the measurement of phosphorylated tau levels; and (c) administering the therapeutic drug to the subject.

In embodiments, the present disclosure provides a method of distinguishing a subject afflicted with Alzheimer's disease from an individual afflicted with non-Alzheimer's dementia, the method comprising: (a) obtaining a measurement of phosphorylated tau levels of the subject as described herein; and (b) identifying, based on the measurement of phosphorylated tau levels, the subject as (i) afflicted with Alzheimer's disease or (ii) afflicted with non-Alzheimer's dementia.

In embodiments, the present disclosure provides a method of treating Alzheimer's disease in a subject in need thereof, the method comprising: (a) obtaining a measurement of phosphorylated tau levels of the subject as described herein, wherein the measurement is obtained prior to administration of a treatment for Alzheimer's disease, (b) determining, based on the measurement of phosphorylated tau levels, that the subject is afflicted with Alzheimer's disease, and (c) administering a treatment regimen for Alzheimer's disease to the subject.

In embodiments, the present disclosure provides a method of monitoring response to treatment for Alzheimer's disease in a subject, the method comprising: (a) obtaining a first measurement of phosphorylated tau levels of the subject as described herein, wherein the first measurement is obtained prior to administration of a treatment regimen for Alzheimer's disease, (b) obtaining a second measurement of phosphorylated tau levels of the subject at one or more time points after administration of the treatment regimen for Alzheimer's disease has been initiated, (c) determining, based on the first and second measurements of phosphorylated tau levels, that the subject is responding positively to the Alzheimer's treatment regimen, and (d) continuing to administer the treatment regimen for Alzheimer's disease to the subject.

The methods described herein provide an accurate method of determining cognitive impairment progression in an individual by utilizing a simple, convenient, sensitive, and specific method with a wide dynamic range for detecting the amount of p-tau, total tau, or brain-associated tau. Unexpectedly, the methods provided herein are capable of accurately distinguishing between (i) cognitively normal individuals who do not progress to any cognitive impairment during their lifetime (i.e., individuals whose diagnoses or symptoms remain “stable”), and (ii) cognitively normal individuals who later progress to mild cognitive impairment (MCI) during their lifetime (i.e., individuals whose diagnoses or symptoms “decline”). Advantageously, the methods provided herein are capable of determining an individual as being likely to experience cognitive decline even before the cognitive decline is detected by neurocognitive testing and/or PET imaging. Further, the methods provided herein are capable of accurately distinguishing between (i) individuals with mild MCI who do not progress to any further cognitive impairment during their lifetime (stable), and (ii) individuals with mild MCI who later progress to dementia during their lifetime (decline). In embodiments, the methods provided herein are capable of accurately distinguishing between (i) individuals with SCC who do not progress to any further cognitive impairment during their lifetime (stable), and (ii) individuals with SCC who later progress to dementia during their lifetime (decline). In embodiments, the methods provided herein are capable of accurately distinguishing between (i) cognitively normal individuals and (ii) individuals who have AD. In embodiments, the methods provided herein are capable of accurately distinguishing between (i) individuals who have AD and (ii) individuals who have a non-AD neurodegenerative disease. In embodiments, the non-AD neurodegenerative disease is frontotemporal dementia, progressive supranuclear palsy, Lewy body dementia, Pick's disease, cerebrovascular disease, amyotrophic lateral sclerosis, corticobasal degeneration, Creutzfeldt-Jakob disease, cerebral amyloid angiopathy, multiple sclerosis, thalamic degeneration, or dementia lacking distinctive histology. In contrast with prior studies referenced herein that only assess a patient population's likelihood of cognitive impairment, the methods herein are capable of providing both a cognitive impairment likelihood assessment of an individual and of a patient population. In embodiments, the methods herein monitor or track an individual subject's tau levels, e.g., p-tau or tau, e.g., once every 1 month, 3 months, 6 months, 9 months, 1 year, 2 years, 3 years, 5 years, or more, over the course of 1 year, 2 years, 3 years, 4 years, 5 years, 6 years, 7 years, 8 years, 9 years, 10 years, or more than 10 years, or over the course of the subject's lifetime. For example, an individual subject can be tested every 1, 2, 3, 4, 5, 6, 7, 8, 9, 10, or more years using the methods described herein to screen for an increased risk of developing MCI and/or AD and/or increased risk of cognitive decline. In embodiments, the methods track the individual's tau levels, e.g., p-tau or tau, thereby allowing for early detection, intervention, and treatment of AD. In embodiments, the p-tau comprises pTau217 and pTau231.

The present methods are advantageously capable of distinguishing between patient population groups with high accuracy. As used herein, a “normalized” concentration of a particular biomarker, e.g., p-tau, tau or brain-associated tau, is the average concentration of the biomarker as measured in a population. In embodiments, when comparing biomarker levels of a subject to a normalized concentration, the normalized concentration is determined in a cohort to which the subject belongs. In embodiments, the cohort is determined based on the subject's gender, age group, preexisting health condition(s), family history, levels of other biomarkers, or combinations thereof. In embodiments, the normalized p-tau concentration is normalized for age and sex of the cohort. In embodiments, the p-tau comprises pTau217 and pTau231.

In embodiments, the disclosure provides a method of detecting and/or quantifying p-tau in a sample, comprising: (a) contacting the sample with: (i) a capture reagent that binds a phosphorylated tau site or a non-phosphorylated tau site; (ii) a first detection reagent that binds a phosphorylated tau site or a non-phosphorylated tau site; and (iii) a second detection reagent that binds a phosphorylated tau site or a non-phosphorylated tau site; (b) thereby forming a complex comprising the capture reagent, p-tau, and the first and second detection reagents; and (c) detecting and/or quantifying the complex, thereby detecting and/or quantifying p-tau, wherein at least two of the capture reagent, first detection reagent and second detection reagent bind to a phosphorylated tau site of SEQ ID NO: 1, 2 or 3. In embodiments, at least one of the capture reagent, first detection reagent and second detection reagent binds a brain-associated tau polypeptide site of SEQ ID NO: 2 or 3. In embodiments, the sample is from a subject diagnosed with Alzheimer's disease (AD) or at risk of developing AD. In embodiments, the p-tau comprises pTau217 and pTau231.

The methods herein can be used to assess with high clinical confidence the likelihood of developing Alzheimer's Disease (AD) and/or cognitive decline in cognitively normal individuals or individuals, which allows for early intervention and/or treatment. In embodiments, the cognitive decline referred to herein is more than that predicted from the individual's age. The methods herein further accurately distinguish between individuals with AD and individuals with non-AD dementia. As discussed herein, early and accurate detection and diagnosis of AD can be highly important for proper treatment and/or selection for participation in clinical trials.

In embodiments, the disclosure provides a method of determining if a subject is likely to experience cognitive decline during the subject's lifetime. In embodiments, the disclosure provides a method of determining if a subject is likely to experience cognitive decline within 1 to 50 years, 1 to 40 years, 1 to 30 years, 2 to 25 years, 3 to 20 years, 4 to 15 years, or 5 to 10 years. In embodiments, the disclosure provides a method of determining if a subject is likely to experience cognitive decline within about 1, 2, 3, 4, 5, 6, 7, 8, 9, 10, 11, 12, 13, 14, 15, 16, 17, 18, 19, 20, or more than 20 years, or over the course of the subject's lifetime.

In embodiments, the disclosure provides a method of determining if a cognitively normal subject is likely to experience cognitive decline within about 1 to about 20 years, or about 1 to about 10 years, or about 1 to about 5 years, or about 1, 2, 3, 4, 5, 6, 7, 8, 9, or 10 years, comprising: (a) obtaining a measurement of p-tau and/or total tau levels of the subject; and (b) determining if the subject is likely to experience cognitive decline based on the measurement of p-tau and/or total tau levels. In embodiments, the disclosure provides a method of preventing, reducing, or delaying cognitive decline in a cognitively normal subject, comprising: (a) obtaining a measurement of p-tau and/or total tau levels of the subject; (b) identifying the subject as being likely to experience future cognitive decline, e.g., within about 1 to about 20 years, or about 1 to about 10 years, or about 1 to about 5 years, or about 1, 2, 3, 4, 5, 6, 7, 8, 9, or 10 years, based on the measurement of p-tau levels; and (c) administering a regimen to the subject to prevent, reduce, or delay cognitive decline. In embodiments, step (a) comprises obtaining a measurement of the subject's p-tau levels. In embodiments, step (a) comprises obtaining a measurement of the subject's total tau levels. In embodiments, step (a) comprises obtaining a measurement of the levels of both p-tau and total tau. In embodiments, the measurement of p-tau and/or total tau levels is measured by a method described herein. In embodiments, the method comprises determining that the cognitively normal subject is likely to experience further cognitive decline within about 1 to about 5 years, or about 1 to about 4 years, or about 1 to about 3 years, or about 1 to about 2 years, when the subject's p-tau levels are at least 1.5-fold, at least 1.6-fold, at least 1.7-fold, at least 1.8-fold, at least 1.9-fold, at least 2-fold, at least 3-fold, at least 4-fold, or at least 5-fold higher than a normalized p-tau concentration; and/or when the subject's total tau levels are at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 3-fold, at least 4-fold, or at least 5-fold higher than a normalized total tau concentration. In embodiments, the p-tau comprises pTau217 and pTau231.

In embodiments, the disclosure provides a method of determining if a subject is likely to experience cognitive decline within about 1 to about 20 years, or about 1 to about 10 years, or about 1 to about 5 years, or about 1, 2, 3, 4, 5, 6, 7, 8, 9, or 10 years, comprising: (a) obtaining a measurement of p-tau and/or total tau levels of the subject; and (b) determining if the subject is likely to experience cognitive decline based on the measurement of p-tau and/or total tau levels. In embodiments, the disclosure provides a method of preventing, reducing, or delaying cognitive decline in a subject with subjective cognitive complaints, comprising: (a) obtaining a measurement of p-tau and/or total tau levels of the subject; (b) identifying the subject as being likely to experience future cognitive decline, e.g., within about 1 to about 20 years, or about 1 to about 10 years, or about 1 to about 5 years, or about 1, 2, 3, 4, 5, 6, 7, 8, 9, or 10 years, based on the measurement of p-tau and/or total tau levels; and (c) administering a regimen to the subject to prevent, reduce, or delay cognitive decline. In embodiments, step (a) comprises obtaining a measurement of the subject's p-tau levels. In embodiments, step (a) comprises obtaining a measurement of the subject's total tau levels. In embodiments, step (a) comprises obtaining a measurement of the levels of both p-tau and total tau. In embodiments, the measurement of p-tau and/or total tau levels is measured by a method described herein. In embodiments, the method comprises determining that the subject is likely to experience cognitive decline within about 1 to about 5 years, or about 1 to about 4 years, or about 1 to about 3 years, or about 1 to about 2 years, when the subject's p-tau and/or total tau levels are at least 1.5-fold, at least 1.6-fold, at least 1.7-fold, at least 1.8-fold, at least 1.9-fold, at least 2-fold, at least 3-fold, at least 4-fold, or at least 5-fold higher than a normalized p-tau concentration. In embodiments, the p-tau comprises pTau217 and pTau231.

In embodiments, the disclosure provides a method of distinguishing a subject afflicted with AD from an individual afflicted with non-Alzheimer's dementia, the method comprising: (a) obtaining a measurement of p-tau and/or total tau levels of the subject; and (b) identifying, based on the measurement of p-tau and/or total tau levels, the subject as (i) afflicted with AD or (ii) afflicted with non-Alzheimer's dementia. In embodiments, the disclosure provides a method of treating AD in a subject in need thereof, comprising: (a) obtaining a measurement of p-tau and/or total tau levels of the subject, wherein the measurement is obtained prior to administration of a treatment for AD; (b) determining, based on the measurement of p-tau and/or total tau levels, that the subject is afflicted with Alzheimer's disease; and (c) administering a treatment regimen for AD to the subject. In embodiments, step (a) comprises obtaining a measurement of the subject's p-tau levels. In embodiments, step (a) comprises obtaining a measurement of the subject's total tau levels. In embodiments, step (a) comprises obtaining a measurement of the levels of both p-tau and total tau. In embodiments, the measurement of p-tau and/or tau levels is measured by a method described herein. In embodiments, the method comprises determining that the subject is afflicted with AD when the subject's p-tau levels are at least 2-fold, at least 2.5-fold, at least 3-fold, at least 4-fold, or at least 5-fold higher than a normalized p-tau concentration; and/or when the subject's total tau levels are at least 1.5-fold, at least 1.6-fold, at least 1.7-fold, at least 1.8-fold, at least 1.9-fold, or at least 2-fold higher than a normalized total tau concentration. In embodiments, the method comprises determining that the subject is afflicted with non-Alzheimer's dementia when the subject's p-tau levels are about 1.1-fold to about 1.6-fold, or about 1.2-fold to about 1.5-fold, or about 1.3-fold to about 1.4-fold higher than a normalized p-tau concentration; and/or when the subject's total tau levels are about 1.1-fold to about 1.4-fold, or about 1.1-fold to about 1.3-fold, or about 1.1-fold to about 1.2-fold higher than a normalized total tau concentration. In embodiments, a subject afflicted with AD has at least 1.5-fold, at least 1.6-fold, at least 1.7-fold, at least 1.8-fold, at least 1.9-fold, or at least 2-fold higher p-tau levels and/or at least 1.2-fold, at least 1.3-fold, at least 1.4-fold, or at least 1.5-fold higher total tau levels as compared to an individual afflicted with non-Alzheimer's dementia. In embodiments, the method is capable of distinguishing a subject afflicted with AD from an individual afflicted with non-Alzheimer's dementia, based on p-tau levels of the subject, with a clinical sensitivity of at least 75%, at least 78%, at least 80%, at least 85%, at least 88%, or at least 90%, and a clinical specificity of at least 75%, at least 78%, at least 80%, at least 85%, at least 88%, or at least 90%. In embodiments, the method is capable of distinguishing a subject afflicted with AD from an individual afflicted with non-Alzheimer's dementia, based on total tau levels of the subject, with a clinical sensitivity of at least 60%, at least 65%, at least 70%, at least 75%, at least 80%, at least 85%, or at least 90%, and a clinical specificity of at least 60%, at least 65%, at least 70%, at least 75%, at least 80%, at least 85%, or at least 90%. In embodiments, the p-tau comprises pTau217 and pTau231.

In embodiments, the disclosure provides a method of monitoring response to treatment for AD in a subject, comprising: (a) obtaining a first measurement of p-tau and/or total tau levels of the subject, wherein the first measurement is obtained prior to administration of a treatment regimen for AD; (b) obtaining a second measurement of p-tau and/or total tau levels of the subject at one or more time points after administration of the treatment regimen for AD has been initiated; (c) determining, based on the first and second measurements of p-tau and/or total tau levels, that the subject is responding positively to the treatment regimen; and (d) continuing to administer the treatment regimen for AD to the subject. In embodiments, the one or more time points comprises about 1 hour to about 20 years, about 1 hour to about 10 years, about 12 hours to about 5 years, about 1 day to about 4 years, about 3 days to about 3 years, about 5 days to about 2 years, about 7 days to about 18 months, about 10 days to about 12 months, about 2 weeks to about 11 months, about 3 weeks to about 10 months, about 1 month to about 9 months, about 2 months to about 8 months, about 3 months to about 7 months, or about 4 months to about 6 months. In embodiments, the one or more time points comprises 1, 2, 3, 4, 5, 6, 7, 8, 9, 10, or more than 10 separate time points within about 1 week, within about 1 month, within about 3 months, within about 6 months, within about 1 year, within about 5 years, or within about 10 years. Methods of assessing whether a subject is responding positively to a treatment regimen include, e.g., assessing the change in the subject's biomarker levels (e.g., p-tau and/or total tau) before and after the treatment using a method described herein. In embodiments, the methods provided herein for measuring biomarker levels (e.g., p-tau and/or total tau) provides a simpler and more cost-effective method for assessing a subject's response to treatment as compared to conventional methods, such as imaging studies or long-term evaluation of clinical symptoms. In embodiments, the p-tau comprises pTau217 and pTau231.

In embodiments, the disclosure provides a method of determining eligibility of a subject to participate in a clinical trial of a therapeutic drug for preventing or delaying AD, the method comprising: (a) obtaining a measurement of p-tau and/or total tau levels of the subject; (b) determining the eligibility of the subject for the clinical trial based on the measurement of p-tau and/or total tau levels. In embodiments, the subject has been diagnosed with dementia or MCI. In embodiments, the subject has SCC. In embodiments, the subject does not have any cognitive impairment. In embodiments, the disclosure provides a method of conducting a clinical trial of a therapeutic drug or intervention for Alzheimer's disease, the method comprising: (a) obtaining a measurement of p-tau and/or total tau levels of a subject; (b) determining eligibility of the subject for the clinical trial based on the measurement of p-tau and/or total tau levels; and (c) administering the therapeutic drug to the subject. In embodiments, step (a) comprises obtaining a measurement of the subject's p-tau levels. In embodiments, step (a) comprises obtaining a measurement of the subject's total tau levels. In embodiments, step (a) comprises obtaining a measurement of the levels of both p-tau and total tau. In embodiments, the measurement of p-tau and/or total tau levels is measured by a method described herein. A subject's eligibility in a clinical trial may be determined based on the subject's measured levels of p-tau and/or total tau. In embodiments, a subject is identified as eligible for a clinical trial when the subject's p-tau is greater than 120%, greater than 150%, greater than 170%, or greater than 200% of a normalized p-tau concentration. In embodiments, a subject is identified as ineligible for a clinical trial when the subject's p-tau is less than 100%, less than 90%, less than 85%, less than 80%, or less than 75% of a normalized p-tau concentration. In embodiments, an individual determined to be at low risk for cognitive impairment (e.g., when the subject's p-tau is less than 100%, less than 90%, less than 85%, less than 80%, or less than 75% of a normalized p-tau concentration) is included in a clinical trial as part of the clinical trial's safety evaluation (e.g., an FDA Phase I clinical trial); as a healthy control subject (e.g., an FDA Phase II and/or III clinical trial); and/or as a subject in a long-term study clinical trial (e.g., an FDA Phase IV clinical trial). Normalized concentrations are further described herein. In embodiments, the p-tau comprises pTau217 and pTau231.

In embodiments, the disclosure provides a method of diagnosing AD in a subject comprising, when levels of p-tau in the subject are higher than 120% relative to a normalized concentration of p-tau as measured by a method described herein, diagnosing the subject with AD. In embodiments, when levels of p-tau are less than 50% relative a normalized concentration of p-tau as measured by a method described herein, the method comprises diagnosing the subject as not likely to develop AD. In embodiments, the disclosure provides a method of assessing risk for developing AD in a subject comprising: when levels of p-tau in the subject are higher than 120% relative to a normalized concentration of p-tau as measured by a method described herein, diagnosing the subject as having increased risk of developing AD; and when levels of p-tau in the subject are lower than 50% relative to a normalized concentration of p-tau as measured by a method described herein, diagnosing the subject as having decreased risk of developing AD. In embodiments, the disclosure provides a method of preventing, reducing, or delaying AD in a subject comprising, when levels of p-tau in the subject are higher than 120% relative to a normalized concentration of p-tau as measured by a method described herein, providing a regimen to the subject to prevent, reduce, or delay AD. In embodiments, p-tau levels of higher than 120% relative a normalized concentration of p-tau corresponds to a positive likelihood ratio of about 5 for a method described herein. In embodiments, p-tau levels of lower than 50% relative a normalized concentration of p-tau corresponds to a negative likelihood ratio of about 0.1 for a method described herein. Positive and negative likelihood ratios are further described herein. In embodiments, the p-tau comprises pTau217 and pTau231.

In embodiments, the disclosure provides a method of diagnosing AD in a subject having increased likelihood of developing AD comprising, when levels of p-tau in the subject correspond to a positive likelihood ratio of greater than 5 when determined by a method described herein, diagnosing the subject with AD. In embodiments where a subject has an increased likelihood of developing AD, e.g., due to the subject's age, gender, preexisting health conditions, family history, and/or other biomarker levels, a lower-fold increase of p-tau levels is required for providing an AD diagnosis, as compared to a subject who does not an increased likelihood of developing AD. In embodiments, the disclosure provides a method of assessing risk of developing AD in a subject having increased likelihood of developing AD comprising: when levels of p-tau in the subject provide a positive likelihood ratio of greater than 5 when determined by a method described herein, determining the subject as having increased risk of developing AD; and when levels of p-tau in the subject provide a negative likelihood ratio of 0.1 when determined by a method described herein, determining the subject as having decreased risk of developing AD. In embodiments, when a subject has increased likelihood of developing AD, p-tau levels in the subject of higher about 105%, higher than about 110%, higher than about 115%, or higher than about 120% relative to a normalized concentration of p-tau correspond to a positive likelihood ratio of about 5 for a method described herein. In embodiments, the disclosure provides a method of preventing, reducing, or delaying AD in a subject having increased likelihood of developing AD comprising, when levels of p-tau in the subject correspond to a positive likelihood ratio of greater than 5 when determined by a method described herein, providing a regimen to the subject to prevent, reduce, or delay AD. In embodiments, the subject having increased likelihood of developing AD is 1.5×, 2×, 3×, 4×, 5×, 6×, 7×, 8×, 9×, or 10× as likely to develop AD within about 1 to about 50 years, or about 1 to about 20 years, or about 1 to about 10 years, or about 1, 2, 3, 4, 5, 6, 7, 8, 9, or 10 years as compared to a cognitively normal subject. In embodiments, the cognitively normal subject is from a same cohort as the subject having increased likelihood of developing AD. In embodiments, a subject is identified as having increased likelihood of AD due to their gender, age group, preexisting health condition(s), family history, prior diagnostic testing (e.g., levels of other biomarkers), or combinations thereof. In embodiments, the p-tau comprises pTau217 and pTau231.

Treatment regimens for AD are known to one of ordinary skill in the art. In embodiments, the treatment regimen for AD comprises one or more drugs that slow disease progression and/or mitigate one or more symptoms of AD, including cognitive and non-cognitive symptoms. An exemplary drug that slows AD progression is aducanumab, also known by its tradename ADUHELM™. Non-limiting examples of drugs for mitigation of AD cognitive symptoms include cholinesterase inhibitors, e.g., donepezil (ARICEPT®), rivastigmine (EXELON®), and galantamine (RAZADYNE®); glutamate regulators, e.g., memantine (NAMENDA®); or combination therapies such as NAMZARIC®, a combination of donepezil and memantine. Non-limiting examples of drugs for mitigation of AD behavioral and psychological symptoms include orexin receptor antagonists, e.g., suvorexant (BELSOMRA®). Further drugs that may be comprised in the treatment regimen for AD include, for example, sleep aids, anti-anxiety drugs, anti-convulsants, and/or antipsychotics.

In embodiments, the treatment regimen for AD comprises a non-drug therapeutic, e.g., light therapy using a photobiomodulation (PBM) device as described in Dougal et al., Photomodulation, Photomedicine, and Laser Surgery 39(10):654-660 (2021). In embodiments, the treatment regimen for AD comprises treatments of sleep disorders, e.g., bright light and melatonin therapy, anti-depressant hypnotic therapy, continuous positive airway pressure, acoustic stimulation, and transcranial alternating current stimulation as described in Kent et al., Progress in Neurobiology 197:101902 (2021). In embodiments, the treatment regimen for AD comprises active and passive immunotherapy treatments, e.g., anti-Ap immunotherapies as described in Spencer and Masliah, Frontiers in Aging Neuroscience, 6:114 (2014). In embodiments, the treatment regimen for AD comprises stem cell therapies using embryonic stem cells, induced pluripotent stem cells, mesenchymal stem cells treatments, or combinations thereof as described in Athar et al., Molecular Biology Reports 48(7):5629-5645 (2021). In embodiments, the treatment regimen for AD comprises therapeutics targeting secretases including 8-secretase inhibitors, γ-secretase inhibitors, α-secretase stimulators as described in Athar et al., (2021). In embodiments, the treatment regimen for AD comprises treatments targeting exosomes as described in Zhang et al., Ageing Research Reviews 68:101321 (2021).

### Kits

In embodiments, the present disclosure provides a kit for detecting, quantifying, or both, tau in a biological sample, where the tau comprises at least one epitope comprising a first site, wherein the kit comprises: (a) a first binding reagent that binds to a phosphorylated state of the first site; and (b) a second binding reagent that binds to a non-phosphorylated state of the first site. In embodiments, the first binding reagent and/or the second binding reagent is an antibody or antigen-binding fragment thereof. In embodiments, the first binding reagent is a capture reagent and/or the second binding reagent is a detection reagent. In embodiments, the first site comprises any one of the eighty-five serine (S), threonine (T), and tyrosine (Y) tau sites capable of being phosphorylated. In embodiments, the first site is selected from: T175, T181, T212, T205, 5214, T217, cis or trans T231, 5293, or S396 of SEQ ID NO: 1. In embodiments, the kit further comprises a surface.

In embodiments, the present disclosure provides a kit of detecting, quantifying, or both, tau in a biological sample, wherein the tau comprises a plurality of epitopes, the kit comprising: (a) a first binding reagent that binds to a first epitope; (b) a second binding reagent that binds to a second epitope; and (c) a third binding reagent that binds to a third epitope. In embodiments, the first epitope comprises a first site capable of being phosphorylated. In embodiments, the second epitope comprises a second site capable of being phosphorylated. In embodiments, the third epitope comprises a third site capable of being phosphorylated. In embodiments, the first epitope comprises a first site commonly phosphorylated. In embodiments, the second epitope comprises a second site commonly phosphorylated. In embodiments, the third epitope comprises a third site commonly phosphorylated. In embodiments, the first epitope comprises a first site rarely or never phosphorylated. In embodiments, the second epitope comprises a second site rarely or never phosphorylated. In embodiments, the third epitope comprises a third site rarely or never phosphorylated.

In embodiments, the present disclosure provides a kit for detecting and/or quantifying p-tau in a biological sample, wherein the p-tau is phosphorylated at two or more amino acid positions, comprising, in one or more vials, containers, or compartments, three reagents: (a) a capture reagent that binds a phosphorylated tau site, or a non-phosphorylated tau site; (b) a first detection reagent that binds a phosphorylated tau site, or a non-phosphorylated tau site; and (c) a second detection reagent that binds a phosphorylated tau site, or a non-phosphorylated tau site, wherein at least two of the three reagents bind a phosphorylated tau site.

In embodiments, the present disclosure provides a kit for detecting and/or quantifying p-tau in a biological sample, wherein the p-tau comprises: T175 (pTaul75), T181 (pTau181), T212 (pTau212), T205 (pTau205), S214 (pTau214), T217 (pTau217), cis or trans T231 (pTau231), S293 (pTau293), S396 (pTau396), L243 (Tau243) or any combination thereof, of SEQ ID NO: 1. In embodiments, the p-tau comprises SEQ ID NO: 1, or a fragment of variant thereof. In embodiments, the p-tau comprises SEQ ID NO: 2, or a fragment of variant thereof. In embodiments, the p-tau comprises SEQ ID NO: 3, or a fragment of variant thereof. In embodiments, the p-tau comprises SEQ ID NO: 4, or a fragment of variant thereof. In embodiments, the p-tau comprises SEQ ID NO: 5, or a fragment of variant thereof. In embodiments, the p-tau comprises SEQ ID NO: 6, or a fragment of variant thereof. In embodiments, the p-tau comprises SEQ ID NO: 7, or a fragment of variant thereof. In embodiments, the p-tau comprises SEQ ID NO: 8, or a fragment of variant thereof. In embodiments, the p-tau comprises SEQ ID NO: 9, or a fragment of variant thereof. In embodiments, the capture reagent binds pTau217. In embodiments, the first detection reagent binds pTau231. In embodiments, the second detection reagent binds a third phosphorylated tau site and/or a brain-associated tau polypeptide site comprising SEQ ID NO: 2 or 3.

In embodiments, the present disclosure provides a kit for detecting and/or quantifying brain associated tau polypeptide. In embodiments, the present disclosure provides a kit for detecting and/or quantifying p-tau in a biological sample, wherein the p-tau comprises a brain-associated tau polypeptide sequence. In embodiments, the brain-associated tau polypeptide sequence comprises SEQ ID NO: 2 or 3, or a fragment or variant thereof.

In embodiments, the present disclosure provides a kit, comprising: (a) a capture reagent that binds pTau217; (b) a first detection reagent that binds pTau231; and (c) a second detection reagent that binds pTau181, Tau243, or a brain-associated tau polypeptide comprising the amino acid sequence of SEQ ID NO: 2 (HVTQARMV) or SEQ ID NO: 3 (AGHVTQARMVSK).

In embodiments, the present disclosure provides a kit, comprising: (a) a capture reagent that binds pTau217; (b) a first detection reagent that binds pTau231; and (c) a second detection reagent that binds a brain-associated tau polypeptide comprising the amino acid sequence of SEQ ID NO: 2 (HVTQARMV) or SEQ ID NO: 3 (AGHVTQARMVSK).

In embodiments, the present disclosure provides a kit, comprising: (a) a capture reagent that binds pTau217; (b) a first detection reagent that binds pTau181; and (d) a second detection reagent that binds pTau231.

In embodiments, the present disclosure provides a kit for detecting p-tau in a biological sample, comprising: (a) a single connector oligonucleotide (b) a first nucleic acid probe (NAP1) that binds to 5′ and 3′ ends of the single connector oligonucleotide; (c) a ligase; (d) a capture reagent that binds to phosphorylated or non-phosphorylated tau site; and (e) a detection reagent that binds to phosphorylated or non-phosphorylated tau site, wherein the detection reagent is conjugated to the NAP1, or wherein the kit further comprises a reagent for conjugating the detection reagent to the NAP1; and wherein the single connector oligonucleotide is capable of being ligated to form the circular template oligonucleotide. In embodiments, the kit further comprises a polymerase.

In embodiments, the present disclosure provides a kit for detecting p-tau in a biological sample, comprising: (a) a first connector oligonucleotide (CON1); (b) a second connector oligonucleotide (CON2); (c) a first nucleic acid probe (NAP1) that binds to a 5′ end of the CON1 and to a 3′ end of the CON2; (d) a second nucleic acid probe (NAP2) that binds to a 3′ end of the CON1 and to a 5′ end of the CON2; (e) a ligase; (f) a polymerase; (g) a capture reagent that binds to a phosphorylated or non-phosphorylated first site of the p-tau; (h) a first detection reagent that binds to a phosphorylated or non-phosphorylated second site of the p-tau; and (i) a second detection reagent that binds to a phosphorylated or non-phosphorylated third site of the p-tau. In some embodiments, at least two of the capture reagent, the first detection reagent, and the second detection reagent bind to a phosphorylated site of the p-tau. In embodiments, the CON1 and the CON2 are capable of being ligated to form a circular template oligonucleotide. In embodiments, the first detection reagent is conjugated to the NAP1 and the second detection reagent is conjugated to the NAP2, or the kit further comprises a reagent for conjugating the first detection reagent to the NAP1 and the second detection reagent to the NAP2.

In embodiments, the circular template oligonucleotide comprises about 40% to about 80% thymine (T). In embodiments, the circular template oligonucleotide is about 30 to about 80 bases in length. In embodiments, the circular template oligonucleotide comprises a detection sequence, and one or both of the NAP1 and the NAP2 comprises at least 80% sequence identity to a detection sequence complement. In embodiments, the circular template oligonucleotide comprises at least three detection sequences. In embodiments where the kit comprises the CON1 and the CON2, the CON1 and the CON2 are each about 15 to about 80 bases in length and do not differ by more than 5, 6, or 7 bases in length. In embodiments, the CON1 comprises an anchoring sequence and the CON2 comprises a detection sequence.

In embodiments, the present disclosure provides a kit, wherein the kit further comprises a surface, a detectable label, an anchoring reagent, a labeled probe, an immobilizing reagent, a template oligonucleotide, a polymerase, a ligase, a buffer, a blocking agent, a co-reactant, a diluent, a stabilizing agent, a calibration agent, an assay consumable, an electrode or any combination thereof.

In embodiments, each capture reagent and detection reagent comprises an antibody or antigen-binding fragment thereof.

In embodiments, each capture reagent or detection reagent that binds p-tau does not bind to non-phosphorylated tau.

In embodiments, the capture reagent comprises a targeting reagent that is capable of selectively binding to a corresponding targeting reagent complement immobilized on a binding domain on a surface. In embodiments, the capture reagent comprises a labile linker and is releasably bound to the surface.

In embodiments, the first detection reagent comprises a first nucleic acid probe, and the second detection reagent comprises a second nucleic acid probe. In embodiments, the kit provided herein further comprises a first nucleic acid probe, a second nucleic acid probe, and a reagent for conjugating: the first nucleic acid probe to the first detection reagent, and the second nucleic acid probe to the second detection reagent.

In embodiments, the disclosure provides a kit for detecting p-tau comprising, in one or more vials, containers, or compartments: (a) a capture reagent that binds p-tau; (b) a detection reagent that binds p-tau; and (c) optionally a surface, wherein the capture reagent is provided on the surface or is capable of binding to the surface. In embodiments, the kit comprises the surface. In embodiments, the kit does not comprise a surface.

In embodiments, the disclosure provides a kit for detecting p-tau comprising, in one or more vials, containers, or compartments: (a) a capture reagent that binds p-tau; (b) a first detection reagent that binds p-tau; (c) a second detection reagent that binds p-tau; and (d) optionally a surface, wherein the capture reagent is provided on the surface or is capable of binding to the surface. In embodiments, the kit comprises the surface. In embodiments, the kit does not comprise a surface.

In embodiments, the disclosure provides a kit for detecting total tau comprising, in one or more vials, containers, or compartments: (a) a capture reagent that binds tau; (b) a detection reagent that binds tau; and (c) optionally a surface, wherein the capture reagent is provided on the surface or is capable of binding to the surface. In embodiments, the capture reagent and the detection reagent are capable of binding non-phosphorylated tau and/or p-tau. In embodiments, the kit comprises the surface. In embodiments, the kit does not comprise a surface.

In embodiments, the disclosure provides a kit for detecting p-tau comprising, in one or more vials, containers, or compartments: (a) a capture reagent that binds p-tau; (b) a first detection reagent that binds p-tau; (c) a second detection reagent that binds tau; and (d) optionally a surface, wherein the capture reagent is provided on the surface or is capable of binding to the surface. In embodiments, the capture reagent and the first and second detection reagents are capable of binding non-phosphorylated tau and/or p-tau. In embodiments, the kit comprises the surface. In embodiments, the kit does not comprise a surface.

Capture reagents are described herein. In embodiments, the capture reagent is an antibody, antigen, ligand, receptor, oligonucleotide, hapten, epitope, mimotope, or an aptamer. In embodiments, the capture reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the capture reagent comprises at least one heavy or light chain complementarity determining region (CDR) of an antibody. In embodiments, the capture reagent comprises at least two CDRs from one or more antibodies. In embodiments, the capture reagent is an antibody or antigen-binding fragment thereof.

In embodiments, the capture reagent binds tau. In embodiments, the capture reagent is capable of binding non-phosphorylated tau. In embodiments, the capture reagent is capable of binding p-tau. In embodiments, the capture reagent is capable of binding to both non-phosphorylated tau and p-tau. In embodiments, the capture reagent is capable of binding to p-tau that is phosphorylated at T175, T181, T205, T212, 5214, T217, cis T231, trans T231, S293, S396, of SEQ ID NO: 1, or a combination thereof. In embodiments, the capture reagent is capable of binding to pTau that is phosphorylated at T217. In embodiments, the capture reagent is the antibody MSD clone VEC0983-0981 or MSD clone VEC0728-0727.

In embodiments, the kit comprises a surface, and the capture reagent is immobilized on the surface. In embodiments, the kit comprises a surface, and the capture reagent is capable of being immobilized to the surface. In embodiments, the kit further comprises a reagent for immobilizing the capture reagent to the surface. Immobilization of capture reagents onto surfaces is described herein. In embodiments, the capture reagent is directly immobilized onto the surface. In embodiments, the capture reagent is indirectly immobilized onto the surface, e.g., via secondary binding reagents. Secondary binding reagents, e.g., targeting agents, targeting agent complements, and bridging agents, are further described herein.

In embodiments, the kit comprises a detection reagent. Detection reagents are described herein. In embodiments, the detection reagent is an antibody, antigen, ligand, receptor, oligonucleotide, hapten, epitope, mimotope, or an aptamer. In embodiments, the detection reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the detection reagent comprises at least one heavy or light chain complementarity determining region (CDR) of an antibody. In embodiments, the detection reagent comprises at least two CDRs from one or more antibodies. In embodiments, the detection reagent is an antibody or antigen-binding fragment thereof.

In embodiments where the kit is for detecting pTau231, the detection reagent specifically binds pTau231. In embodiments where the method detects pTau231, the detection reagent binds pTau231 and does not bind non-phosphorylated tau. In embodiments where the method detects pTau231, the detection reagent binds pTau231 and does not bind p-tau that is not phosphorylated at amino acid position T231.

In embodiments where the kit is for detecting pTau181, the detection reagent specifically binds pTau181. In embodiments where the method detects pTau181, the detection reagent binds pTau181 and does not bind non-phosphorylated tau. In embodiments where the method detects pTau181, the detection reagent binds pTau181 and does not bind p-tau that is not phosphorylated at amino acid position T181. Exemplary detection reagents that specifically bind pTau181 include, but are not limited to, the antibodies listed under Invitrogen catalog nos. MN1050 (see, e.g., Meredith Jr. et al., PLoS ONE 8(10): e76523 (2013)) and 701530. In embodiments, the detection reagent is the antibody MSD clone VEC1980-1978.

In embodiments where the kit is for detecting total tau, the detection reagent is capable of binding non-phosphorylated tau and/or p-tau, for example, p-tau that is phosphorylated at any of amino acid positions T175, T181, T212, S214, T217, cis T231, trans T231, S293, S396, or a combination thereof. In embodiments, the detection reagent is the antibody MSD clone VEC1367-1366.

In embodiments, the detection reagent comprises a detectable label. In embodiments, the detection reagent is capable of being conjugated to a detectable label, and the kit further comprises the detectable label and/or a reagent for performing the conjugation. Methods of conjugating detectable labels to detection reagents are known to one of ordinary skill in the art. In embodiments, the detection reagent comprises a first reactive group, and the detectable label comprises a second reactive group that is capable of reacting with the first reactive group. Non-limiting examples of first and second reactive groups include: amine and N-hydroxysuccinimide (NHS) ester; thiol and maleimide; thiol and iodoacetamide; thiol and activated disulfide; alkene or strained alkene and tetrazine; and alkyne or strained alkyne and azide.

Detectable labels are further described herein. In embodiments, the detectable label is capable of being measured by light scattering, optical absorbance, fluorescence, chemiluminescence, electrochemiluminescence (ECL), bioluminescence, phosphorescence, radioactivity, magnetic field, or combinations thereof. In embodiments, the detectable label comprises one or more ECL labels. In embodiments, the detectable label comprises a fluorescent label.

In embodiments, the detection reagent comprises a nucleic acid probe. In embodiments, the detection reagent is capable of being conjugated to a nucleic acid probe, and the kit further comprises the nucleic acid probe and/or a reagent for performing the conjugation. Methods of conjugating nucleic acid probes to detection reagents are known to one of ordinary skill in the art and are described, e.g., in WO 2020/180645. In embodiments, the detection reagent comprises a first reactive group, and the nucleic acid probe comprises a second reactive group that is capable of reacting with the first reactive group. In embodiments, the reagent for performing the conjugation comprises a first reactive group that is capable of reacting with the detection reagent and a second reactive group that is capable of reacting with the nucleic acid probe. Examples of reactive groups and their reaction partners include but are not limited to: amine and N-hydroxysuccinimide (NHS) ester; thiol and maleimide; thiol and iodoacetamide; thiol and activated disulfide; alkene or strained alkene and tetrazine; and alkyne or strained alkyne and azide.

In embodiments, the detection reagent comprises a nucleic acid probe, and the kit further comprises an anchoring reagent. Anchoring reagents are described herein. In embodiments, the kit comprises a surface, and the anchoring reagent is provided on the surface. In embodiments, the kit comprises a surface, and the anchoring reagent is capable of being immobilized to the surface. In embodiments, the kit further comprises a reagent for immobilizing the anchoring reagent on the surface. Immobilization of anchoring reagents to surfaces are described herein. In embodiments, the anchoring reagent is directly immobilized onto the surface. In embodiments, the anchoring reagent is indirectly immobilized onto the surface, e.g., via secondary binding reagents. Secondary binding reagents, e.g., targeting agents and targeting agent complements, are further described herein.

In embodiments, the detection reagent comprises a nucleic acid probe, and the kit further comprises a template oligonucleotide and/or a polymerase. Template oligonucleotides and polymerases, e.g., for performing the PCR, LCR, SDA, 3SR, and/or isothermal amplification (such as, e.g., helicase-dependent amplification or RCA), are described herein. In embodiments, the kit further comprises a ligase, e.g., for ligating the template oligonucleotide. In embodiments, the template oligonucleotide is capable of hybridizing to the nucleic acid probe. In embodiments, the template oligonucleotide comprises a first region comprising a same sequence as the anchoring reagent. In embodiments, the kit further comprises a labeled probe. Labeled probes are further described herein. In embodiments, the labeled probe comprises a detection oligonucleotide, and the template oligonucleotide comprises a second region comprising a same sequence as the detection oligonucleotide. In embodiments, the labeled probe comprises a detectable label. In embodiments, the detectable label is capable of being measured by light scattering, optical absorbance, fluorescence, chemiluminescence, electrochemiluminescence (ECL), bioluminescence, phosphorescence, radioactivity, magnetic field, or combinations thereof. In embodiments, the detectable label comprises one or more ECL labels. Detectable labels are further described herein. In embodiments, the detectable label comprises a fluorescent label.

In embodiments, the kit comprises a first detection reagent and a second detection reagent. First and second detection reagents are described herein. In embodiments, the first detection reagent and the second detection reagent are each independently an antibody, antigen, ligand, receptor, oligonucleotide, hapten, epitope, mimotope, or an aptamer. In embodiments, the first detection reagent and the second detection reagent are each independently an antibody or a variant thereof, including an antigen/epitope-binding portion thereof, an antibody fragment or derivative, an antibody analogue, an engineered antibody, or a substance that binds to antigens in a similar manner to antibodies. In embodiments, the first detection reagent and the second detection reagent each independently comprises at least one heavy or light chain complementarity determining region (CDR) of an antibody. In embodiments, the first detection reagent and the second detection reagent each independently comprises at least two CDRs from one or more antibodies. In embodiments, the first detection reagent and the second detection reagent are each independently an antibody or antigen-binding fragment thereof.

In embodiments, the first detection reagent binds tau. In embodiments, the first detection reagent is capable of binding to non-phosphorylated tau. In embodiments, the first detection reagent is capable of binding to p-tau. In embodiments, the first detection reagent is capable of binding to both non-phosphorylated tau and p-tau. In embodiments, the first detection reagent is capable of binding to p-tau that is phosphorylated at T175, T181, T212, S214, T217, cis T231, trans T231, S293, S396, or a combination thereof. In embodiments, the first detection reagent is capable of binding to p-tau that is phosphorylated at T181. In embodiments, the first detection reagent is capable of binding to p-tau that is phosphorylated at T231.

In embodiments where the kit is for detecting pTau181, the second detection reagent specifically binds pTau181. In embodiments where the kit is for detecting pTau181, the second detection reagent binds pTau181 and does not bind non-phosphorylated tau. In embodiments where the kit is for detecting pTau181, the second detection reagent binds pTau181 and does not bind pTau that is not phosphorylated at amino acid position T181.

In embodiments where the kit is for detecting total tau, the second detection reagent is capable of binding non-phosphorylated tau and/or p-tau, for example, p-tau that is phosphorylated at any of amino acid positions T175, T181, T205, T212, S214, T217, cis T231, trans T231, S293, S396, or a combination thereof.

In embodiments, the first detection reagent and/or the second detection reagent comprises a detectable label. In embodiments, the first detection reagent and/or the second detection reagent is capable of being conjugated to a detectable label, and the kit further comprises the detectable label and/or a reagent for performing the conjugation. Conjugation of detectable labels to detection reagents are described herein. In embodiments, the detectable label is capable of being measured by light scattering, optical absorbance, fluorescence, chemiluminescence, electrochemiluminescence (ECL), bioluminescence, phosphorescence, radioactivity, magnetic field, or combinations thereof. In embodiments, the detectable label comprises one or more ECL labels. In embodiments, the detectable label comprises a fluorescent label.

In embodiments, the first detection reagent comprises a first nucleic acid probe, and the second d detection reagent comprises a second nucleic acid probe. In embodiments, the first detection reagent is capable of being conjugated to a first second nucleic acid probe, the second detection reagent is capable of being conjugated to a second nucleic acid probe, and the kit further comprises the first nucleic acid, the second nucleic acid probe, and/or a reagent for performing the conjugation. Methods of conjugating nucleic acid probes to detection reagents are described herein. In embodiments, the first detection reagent is substantially non-conjugable with the second nucleic acid probe, and the second detection reagent is substantially non-conjugable with the first nucleic acid probe.

In embodiments, the first and second detection reagents comprise first and second nucleic acid probes, and the kit further comprises an anchoring reagent. Anchoring reagents are described herein. In embodiments, the kit comprises a surface, and the anchoring reagent is provided on the surface. In embodiments, the kit comprises a surface, and the anchoring reagent is capable of being immobilized to the surface. Immobilization of anchoring reagents to surfaces are described herein. In embodiments, the anchoring reagent is directly immobilized onto the surface. In embodiments, the anchoring reagent is indirectly immobilized onto the surface, e.g., via secondary binding reagents. Secondary binding reagents, e.g., targeting agents and targeting agent complements, are further described herein.

In embodiments, the first and second detection reagents comprise first and second nucleic acid probes, and the kit further comprises a template oligonucleotide, a first and second connector oligonucleotides, and/or a polymerase. Template oligonucleotides and polymerases, e.g., for performing the PCR, LCR, SDA, 3SR, and/or isothermal amplification (such as, e.g., helicase-dependent amplification or RCA), are described herein. In embodiments, the kit further comprises a ligase, e.g., for ligating the template oligonucleotide. In embodiments, the first and second nucleic acid probes bind to first and second connector oligonucleotides, wherein the first connector oligonucleotide comprises a first connector sequence complementary to a first region of the first nucleic acid probe and a first region of the second nucleic acid probe, and the second connector oligonucleotide comprises a second connector sequence complementary to a second region of the first nucleic acid probe and a second region of the second nucleic acid probe, wherein the first and second regions on each of the first and second nucleic acid probes are non-overlapping; ligating the first and second connector oligonucleotides to form a circular template oligonucleotide; and extending the first and/or second nucleic acid probe, e.g., by RCA. In embodiments, the template oligonucleotide is capable of hybridizing to the first and/or second nucleic acid probes. In embodiments, the template oligonucleotide comprises a first region comprising a same sequence as the anchoring reagent. In embodiments, the kit further comprises a labeled probe. Labeled probes are further described herein. In embodiments, the labeled probe comprises a detection oligonucleotide, and the template oligonucleotide comprises a second region comprising a same sequence as the detection oligonucleotide. In embodiments, the labeled probe comprises a detectable label. In embodiments, the detectable label is capable of being measured by light scattering, optical absorbance, fluorescence, chemiluminescence, electrochemiluminescence (ECL), bioluminescence, phosphorescence, radioactivity, magnetic field, or combinations thereof. In embodiments, the detectable label comprises one or more ECL labels. In embodiments, the detectable label comprises a fluorescent label.

In embodiments, the kits provided herein comprise a surface. In embodiments, a surface is provided separately from the components of the kit. In embodiments, the surface comprises a particle. In embodiments, the surface comprises a well of multi-well plate. In embodiments, the surface comprises a plurality of distinct binding domains. In embodiments, the surface comprises an electrode. In embodiments, the electrode is a carbon ink electrode. Surfaces and methods of immobilizing reagents thereon, e.g., capture and/or anchoring reagents, are further described herein.

In embodiments, one or more components of the kit are lyophilized. In embodiments where the kit comprises a detection reagent, the capture reagent, and/or detection reagent is lyophilized. In embodiments where the kit comprises a detection reagent, the capture reagent, and/or the detection reagent is provided in solution. In embodiments where the kit comprises first and second detection reagents, the capture reagent, the first detection reagent, and/or the second detection reagent is lyophilized. In embodiments where the kit comprises first and second detection reagents, the capture reagent, the first detection reagent, and/or the second detection reagent is provided in solution.

In embodiments, the components of the kit, e.g., the capture and detection reagents and other components, are provided in separate containers, vials, or packages. In embodiments, the components of the kit are provided separately according to each component's optimal shipping and/or storage conditions.

In embodiments, the kit further comprises a polymerase (e.g., a polymerase described herein), a ligase (e.g., a ligase described herein), a calibration reagent, a buffer, a co-reactant, a blocking agent, a diluent, a stabilizing agent, an assay consumable, an electrode, or a combination thereof.

In embodiments, the kit further comprises a calibration reagent. In embodiments, the calibration reagent comprises a known quantity of non-phosphorylated tau, or p-tau. In embodiments, the calibration reagent comprises a recombinant non-phosphorylated tau, or a recombinant p-tau. In embodiments, the recombinant non-phosphorylated tau and/or the recombinant p-tau is expressed in bacteria, e.g., E. coli, or in a mammalian system. In embodiments, the kit comprises multiple calibration reagents comprising a range of concentrations of non-phosphorylated tau or p-tau. In embodiments, the multiple calibration reagents comprise concentrations of non-phosphorylated tau or p-tau, near the upper and lower limits of quantitation for the method. In embodiments, the multiple calibration reagents span the entire dynamic range of the method. In embodiments, the calibration reagent is a positive control reagent. In embodiments, the calibration reagent is a negative control reagent. In embodiments, the positive or negative control reagent is used to provide a basis of comparison for the sample to be tested with the methods of the present disclosure. In embodiments, the calibration reagent is lyophilized. In embodiments, the calibration reagent is provided in solution.

In embodiments, the kit further comprises a buffer, e.g., an assay buffer, a reconstitution buffer, a storage buffer, a read buffer, or a combination thereof. In embodiments, the kit further comprises a co-reactant, e.g., for performing an ECL measurement. Exemplary ECL co-reactants are described, e.g., in WO 2020/142313 and PCT/US21/39835, filed Jun. 30, 2021.

In embodiments, the kit further comprises a blocking agent, e.g., to decrease non-specific binding by components other than tau to the capture and detection reagents described herein. Exemplary blocking agents include, but are not limited to, mBSA, sheared poly(A), polyBSA-I, mIgG, Tween, polyBSA-II, yeast RNA, mBSA+poly(a), and/or polyBSA+poly(A). In embodiments, the kit further comprises a diluent for one or more components of the kit. In embodiments, a kit comprising the components above includes stock concentrations of the components that are 5×, 10×, 20×, 30×, 40×, 50×, 60×, 70×, 80×, 90×, 100×, 125×, 150× or higher fold concentrations of a working concentration for the methods provided herein. In embodiments, the kit further comprises a stabilizing agent, e.g., for storage of one or more components of the kit.

In embodiments, the kit further comprises an assay consumable, e.g., assay modules, vials, tubes, liquid handling and transfer devices such as pipette tips, covers and seals, racks, labels, and the like. In embodiments, the kit further comprises an electrode, e.g., for performing an ECL measurement. In embodiments, the electrode is applied to the surface provided herein. In embodiments, the kit further comprises an assay instrument and/or instructions for carrying out the methods described herein.

In embodiments, the present disclosure provides a system comprising: one or more data processors; and a non-transitory computer readable storage medium containing instructions which, when executed on the one or more data processors, cause the one or more data processors to perform part or all of the methods of detecting and/or quantifying p-tau or tau described herein. In embodiments, the method of detecting, quantifying, or both, p-tau in a biological sample, comprises: (a) contacting the biological sample with a capture reagent, a first detection reagent, and a second detection reagent; (b) forming a complex comprising the capture reagent, the p-tau, the first detection reagent and the second detection reagent; and (c) detecting; or (d) measuring the p-tau, wherein at least two of the capture reagent, first detection reagent, and second detection reagent binds a phosphorylated tau site.

In embodiments, the present disclosure provides a computer-program product tangibly embodied in a non-transitory machine-readable storage medium, including instructions configured to cause one or more data processors to perform part or all of the methods of detecting and/or quantifying p-tau or tau described herein. In embodiments, the method of detecting, quantifying, or both, p-tau in a biological sample, comprises: (a) contacting the biological sample with a capture reagent, a first detection reagent, and a second detection reagent; (b) forming a complex comprising the capture reagent, the p-tau, the first detection reagent and the second detection reagent; and (c) detecting; or (d) measuring the p-tau, wherein at least two of the capture reagent, first detection reagent, and second detection reagent binds a phosphorylated tau site.

In embodiments, the present disclosure provides a non-transitory computer readable medium having stored thereon a computer program which, when executed by a computer system operably connected to an assay system configured to measure, with a three-antibody immunoassay, levels of p-tau in a sample from a human subject, wherein the p-tau is phosphorylated at two or more amino acid positions, causes the computer system to perform a method of determining neurofibrillary tangle counts, a number of brain-associated protein aggregates, or a probability of Alzheimer's Disease (AD) in the human subject, the method comprising: (a) fitting the measured levels of p-tau to a response surface model as a function of the neurofibrillary tangle counts, the number of brain-associated protein aggregates, or the probability of AD; (b) computing a cost function comprising levels of p-tau; and (c) selecting a neurofibrillary tangle count, a number of brain-associated protein aggregates, or AD probability that minimizes the cost function, thereby determining the neurofibrillary tangle counts, the number of brain-associated protein aggregates, or the probability of AD in the human subject. In embodiments, the method of detecting, quantifying, or both, p-tau in a biological sample, comprises: (a) contacting the biological sample with a capture reagent, a first detection reagent, and a second detection reagent; (b) forming a complex comprising the capture reagent, the p-tau, the first detection reagent and the second detection reagent; and (c) detecting; or (d) measuring the p-tau, wherein at least two of the capture reagent, first detection reagent, and second detection reagent binds a phosphorylated tau site.

The assay modules, (e.g., assay plates or cartridges or multi-well assay plates), methods and apparatuses for conducting assay measurements suitable for the present disclosure are described for example, in US 20040022677; US 20050052646; US 20050142033; US 20040189311, US 20140272939, US 20140274775, US 20170168047, US 20190011441, US 20210190778, US 20230407380, US 20170089892, US 20190391140, US 20220357318, US 20140256588, US 20190083976, US 20210402390, US 20160069872, US 20180029034, US 20190291103, and US 20210291168. Assay plates and plate readers are now commercially available (MULTI-SPOT® and MULTI-ARRAY® plates and SECTOR® instruments, MESO SCALE DISCOVERY©).

All references cited herein, including patents, patent applications, papers, textbooks and the like, and the references cited therein, to the extent that they are not already, are hereby incorporated herein by reference in their entirety.

SEQUENCES
amino acid sequence of human CNS tau
protein (2N4R)
SEQ ID NO: 1
MAEPRQEFEVMEDHAGTYGLGDRKDQGGYTMHQDQEGDTDAGLKE
SPLQTPTEDGSEEPGSETSDAKSTPTAEDVTAPLVDEGAPGKQAA
AQPHTEIPEGTTAEEAGIGDTPSLEDEAAGHVTQARMVSKSKDGT
GSDDKKAKGADGKTKIATPRGAAPPGQKGQANATRIPAKTPPAPK
TPPSSGEPPKSGDRSGYSSPGSPGTPGSRSRTPSLPTPPTREPKK
VAVVRTPPKSPSSAKSRLQTAPVPMPDLKNVKSKIGSTENLKHQP
GGGKVQIINKKLDLSNVQSKCGSKDNIKHVPGGGSVQIVYKPVDL
SKVTSKCGSLGNIHHKPGGGQVEVKSEKLDEKDRVQSKIGSLDNI
THVPGGGNKKIETHKLTFRENAKAKTDHGAEIVYKSPVVSGDTSP
RHLSNVSSTGSIDMVDSPQLATLADEVSASLAKQGL;
amino acid sequence of human brain-
associated tau polypeptide
SEQ ID NO: 2
HVTQARMV;
amino acid sequence of human brain-associated
tau polypeptide (for e.g., for immunizations)
SEQ ID NO: 3
AGHVTQARMVSK;
amino acid sequence of human PNS tau
(Big tau) protein:
SEQ ID NO: 4
MAEPRQEFEVMEDHAGTYGLGDRKDQGGYTMHQDQEGDTDAGLKE
SPLQTPTEDGSEEPGSETSDAKSTPTAEDVTAPLVDEGAPGKQAA
AQPHTEIPEGTTAEEAGIGDTPSLEDEAAGHVTQEPESGKVVQEG
FLREPGPPGLSHQLMSGMPGAPLLPEGPREATRQPSGTGPEDTEG
GRHAPELLKHQLLGDLHQEGPPLKGAGGKERPGSKEEVDEDRDVD
ESSPQDSPPSKASPAQDGRPPQTAAREATSIPGFPAEGAIPLPVD
FLSKVSTEIPASEPDGPSVGRAKGQDAPLEFTFHVEITPNVQKEQ
AHSEEHLGRAAFPGAPGEGPEARGPSLGEDTKEADLPEPSEKQPA
AAPRGKPVSRVPQLKARMVSKSKDGTGSDDKKAKTSTRSSAKTLK
NRPCLSPKHPTPGSSDPLIQPSSPAVCPEPPSSPKYVSSVTSRTG
SSGAKEMKLKGADGKTKIATPRGAAPPGQKGQANATRIPAKTPPA
PKTPPSSGEPPKSGDRSGYSSPGSPGTPGSRSRTPSLPTPPTREP
KKVAVVRTPPKSPSSAKSRLQTAPVPMPDLKNVKSKIGSTENLKH
QPGGGKVQIINKKLDLSNVQSKCGSKDNIKHVPGGGSVQIVYKPV
DLSKVTSKCGSLGNIHHKPGGGQVEVKSEKLDFKDRVQSKIGSLD
NITHVPGGGNKKIETHKLTFRENAKAKTDHGAEIVYKSPVVSGDT
SPRHLSNVSSTGSIDMVDSPQLATLADEVSASLAKQGL;
amino acid sequence of human CNS tau
protein (ON3R):
SEQ ID NO: 5
MAEPRQEFEVMEDHAGTYGLGDRKDQGGYTMHQDQEGDTDAGLKA
EEAGIGDTPSLEDEAAGHVTQARMVSKSKDGTGSDDKKAKGADGK
TKIATPRGAAPPGQKGQANATRIPAKTPPAPKTPPSSGEPPKSGD
RSGYSSPGSPGTPGSRSRTPSLPTPPTREPKKVAVVRTPPKSPSS
AKSRLQTAPVPMPDLKNVKSKIGSTENLKHQPGGGKVQIVYKPVD
LSKVTSKCGSLGNIHHKPGGGQVEVKSEKLDFKDRVQSKIGSLDN
ITHVPGGGNKKIETHKLTFRENAKAKTDHGAEIVYKSPVVSGDTS
PRHLSNVSSTGSIDMVDSPQLATLADEVSASLAKQGL;
amino acid sequence of human CNS tau
protein (1N3R):
SEQ ID NO: 6
MAEPRQEFEVMEDHAGTYGLGDRKDQGGYTMHQDQEGDTDAGLKE
SPLQTPTEDGSEEPGSETSDAKSTPTAEAEEAGIGDTPSLEDEAA
GHVTQARMVSKSKDGTGSDDKKAKGADGKTKIATPRGAAPPGQKG
QANATRIPAKTPPAPKTPPSSGEPPKSGDRSGYSSPGSPGTPGSR
SRTPSLPTPPTREPKKVAVVRTPPKSPSSAKSRLQTAPVPMPDLK
NVKSKIGSTENLKHQPGGGKVQIVYKPVDLSKVTSKCGSLGNIHH
KPGGGQVEVKSEKLDFKDRVQSKIGSLDNITHVPGGGNKKIETHK
LTFRENAKAKTDHGAEIVYKSPVVSGDTSPRHLSNVSSTGSIDMV
DSPQLATLADEVSASLAKQGL;
amino acid sequence of human CNS tau
protein (2N3R):
SEQ ID NO: 7
MAEPRQEFEVMEDHAGTYGLGDRKDQGGYTMHQDQEGDTDAGLKE
SPLQTPTEDGSEEPGSETSDAKSTPTAEDVTAPLVDEGAPGKQAA
AQPHTEIPEGTTAEEAGIGDTPSLEDEAAGHVTQARMVSKSKDGT
GSDDKKAKGADGKTKIATPRGAAPPGQKGQANATRIPAKTPPAPK
TPPSSGEPPKSGDRSGYSSPGSPGTPGSRSRTPSLPTPPTREPKK
VAVVRTPPKSPSSAKSRLQTAPVPMPDLKNVKSKIGSTENLKHQP
GGGKVQIVYKPVDLSKVTSKCGSLGNIHHKPGGGQVEVKSEKLDF
KDRVQSKIGSLDNITHVPGGGNKKIETHKLTFRENAKAKTDHGAE
IVYKSPVVSGDTSPRHLSNVSSTGSIDMVDSPQLATLADEVSASL
AKQGL;
amino acid sequence of human CNS tau
protein (ON4R):
SEQ ID NO: 8
MAEPRQEFEVMEDHAGTYGLGDRKDQGGYTMHQDQEGDTDAGLKA
EEAGIGDTPSLEDEAAGHVTQARMVSKSKDGTGSDDKKAKGADGK
TKIATPRGAAPPGQKGQANATRIPAKTPPAPKTPPSSGEPPKSGD
RSGYSSPGSPGTPGSRSRTPSLPTPPTREPKKVAVVRTPPKSPSS
AKSRLQTAPVPMPDLKNVKSKIGSTENLKHQPGGGKVQIINKKLD
LSNVQSKCGSKDNIKHVPGGGSVQIVYKPVDLSKVTSKCGSLGNI
HHKPGGGQVEVKSEKLDFKDRVQSKIGSLDNITHVPGGGNKKIET
HKLTFRENAKAKTDHGAEIVYKSPVVSGDTSPRHLSNVSSTGSID
MVDSPQLATLADEVSASLAKQGL;
amino acid sequence of human CNS tau
protein (1N4R):
SEQ ID NO: 9
MAEPRQEFEVMEDHAGTYGLGDRKDQGGYTMHQDQEGDTDAGLKE
SPLQTPTEDGSEEPGSETSDAKSTPTAEAEEAGIGDTPSLEDEAA
GHVTQARMVSKSKDGTGSDDKKAKGADGKTKIATPRGAAPPGQKG
QANATRIPAKTPPAPKTPPSSGEPPKSGDRSGYSSPGSPGTPGSR
SRTPSLPTPPTREPKKVAVVRTPPKSPSSAKSRLQTAPVPMPDLK
NVKSKIGSTENLKHQPGGGKVQIINKKLDLSNVQSKCGSKDNIKH
VPGGGSVQIVYKPVDLSKVTSKCGSLGNIHHKPGGGQVEVKSEKL
DFKDRVQSKIGSLDNITHVPGGGNKKIETHKLTFRENAKAKTDHG
AEIVYKSPVVSGDTSPRHLSNVSSTGSIDMVDSPQLATLADEVSA
SLAKQGL;

### EXAMPLES

Reference is made to specific examples illustrating the constructs and methods above. It is to be understood that the examples are provided to illustrate rather than limit the scope of various embodiments of the disclosure.

### Example 1. Dual-Antibody Tau Immunoassay

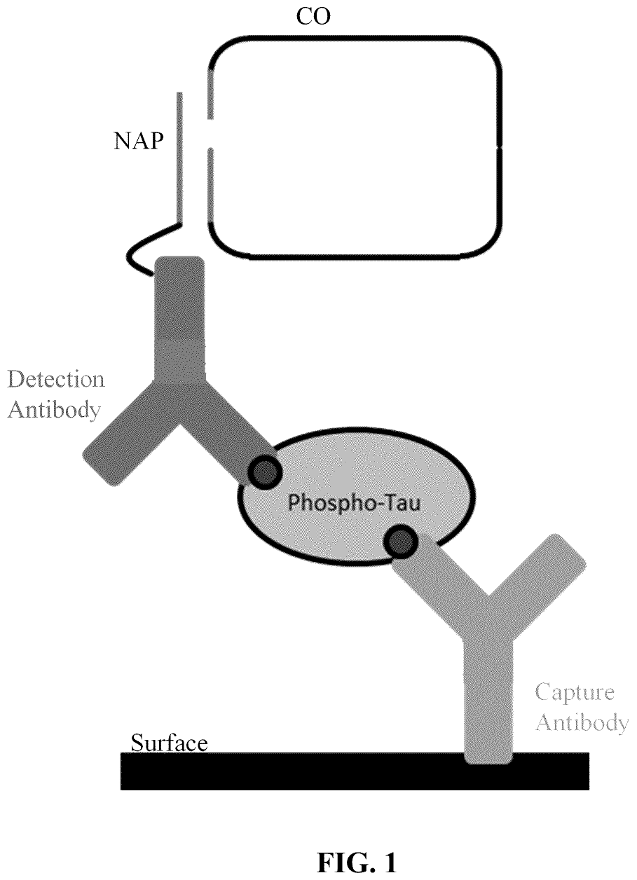

An ultrasensitive immunoassay to measure p-tau, double phosphorylated at amino acid residues 217 and 231, in cerebrospinal fluid (CSF) and plasma utilizes a synergistic combination of a capture antibody, a connector oligonucleotide (CO), and a detection antibody (see FIG. 1). The dual-antibody immunoassay utilizes the following samples: plasma from healthy individuals, plasma from severe traumatic brain injury (sTBI) patients, CSF from healthy individuals, and CSF from sTBI patients. In a validation cohort, double phosphorylation at 231 and 217 in plasma (but not in CSF) compares with phosphorylation at 231 or 217 alone.

The immunoassay comprises a capture antibody that binds pTau217, and a detection antibody that binds pTau231 of p-tau, thereby forming a complex. The detection antibody comprises a nucleic acid probe (NAP) that binds the CO at the 3′ and 5′ terminal regions and ligates the CO to form a circular template oligonucleotide. NAP1 is a primer for an amplification process to form an extended sequences that hybridizes to an anchoring oligonucleotide sequence on the surface. In one embodiment, the capture antibody is immobilized on the surface. In one embodiment, the capture antibody is immobilized on the surface via a linker. The surface can be a bead particle or a multi-well plate. A person of ordinary skill in the art can apply the immunoassay to bind other phosphorylated and non-phosphorylated tau sites using different combinations as presented in Table 1.

### Example 2. Three-Antibody Tau Immunoassay (One Vs. Two Connector Oligonucleotides)

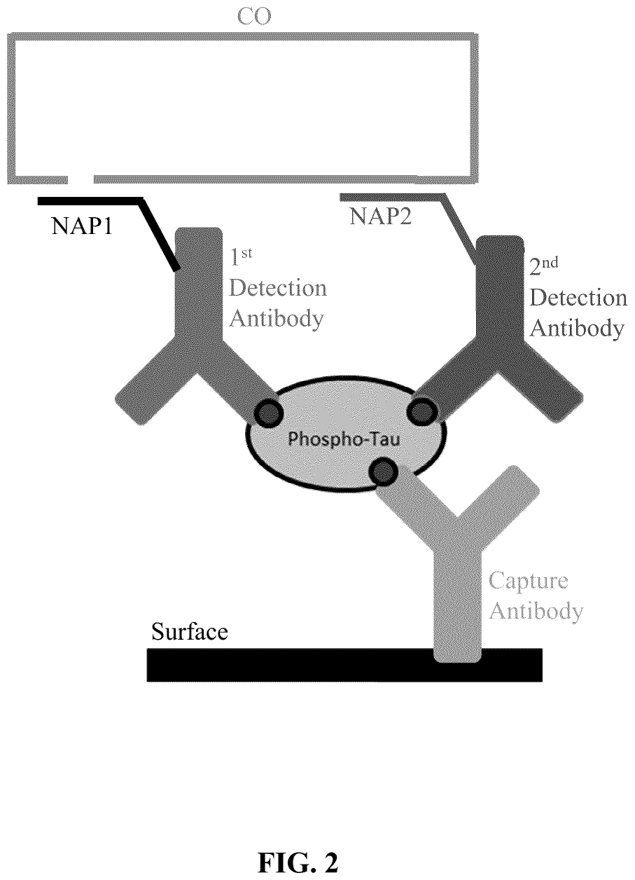

An ultrasensitive immunoassay to measure p-tau, double phosphorylated at amino acid residues 217 and 231, in cerebrospinal fluid (CSF) and plasma utilizes a synergistic combination of one capture and two detection antibodies, hereinafter referred to as a “three-antibody” immunoassay (see FIG. 2). The three-antibody immunoassay utilizes the following samples: plasma from healthy individuals, plasma from severe traumatic brain injury (sTBI) patients, CSF from healthy individuals, and CSF from sTBI patients. In a validation cohort, double phosphorylation at 231 and 217 in plasma (but not in CSF) compares with phosphorylation at 231 or 217 alone.

Each binding domain on the plate comprises a capture antibody and an anchoring moiety (immobilizes as a BSA-oligonucleotide or thiol-oligonucleotide conjugate, the oligonucleotide selected to be specific for a rolling circle amplicon). The immobilized capture antibody binds the p-tau and two detection antibodies bind the same p-tau through two different tau sites. The two detection antibodies are labeled with two different nucleic acid probes (NAPs). A pair of detection antibodies targeting p-tau comprise nucleic acid probes 1 and 2 (NAP1 and NAP2) close in proximity when bound to the p-tau. A ligation mix comprising one or two connector oligonucleotides (CO) comprise nucleotide sequences complimentary to NAP1 and NAP2.

The assay in FIG. 2 uses a single linear CO with one open ligation site to form a circular template oligonucleotide. In this case, the ligation is dependent on the presence of NAP1 and priming is dependent on the presence of NAP2. NAP1 binds and ligates the CO to form a template oligonucleotide, and NAP2 is a primer for an amplification process to form an extended sequences that hybridizes to an anchoring oligonucleotide sequence on the surface.

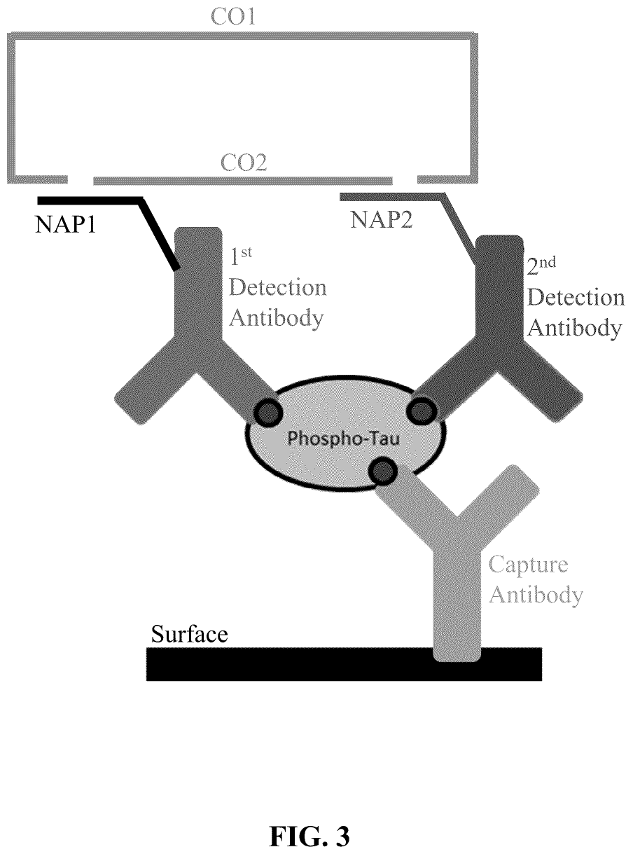

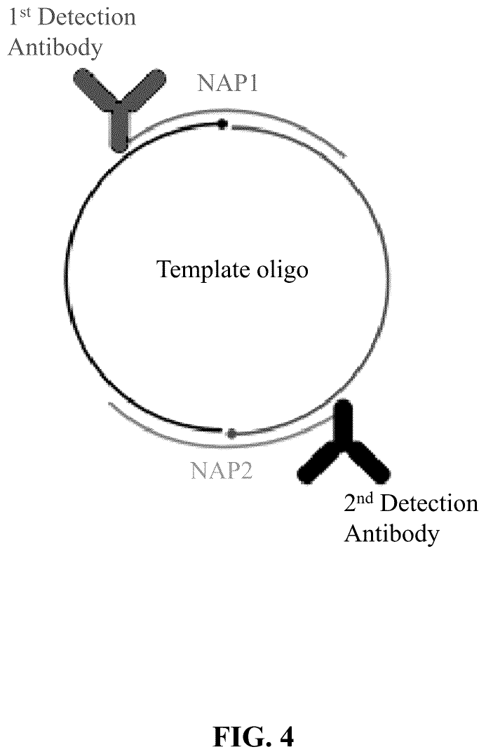

The assay in FIG. 3 uses a three-antibody assay configuration in which two ligation sites are needed to form a circular template oligonucleotide. Upon formation of the complex, NAP1 and NAP2 are in proximity to one another, and each of NAP1 and NAP2 separately binds to both CO1 and CO2 to allow ligation of CO1 and CO2 to form a circular template oligonucleotide. NAP1 and/or NAP2 are primers for an amplification process to form an extended sequence that hybridizes to an anchoring oligonucleotide sequence on the surface. One or both of NAP1 and NAP2 are capable of being extended, thereby forming first and/or second extended sequences as described herein. In one three antibody assay configuration, one of the antibody-conjugated NAPs functions as a primer while the other NAP is chemically blocked and simply functions as a ligation site for the circular DNA. In another configuration, the assay is modified to utilize two priming NAPs (FIG. 4).

The immunoassays using a MESO SCALE DISCOVERY® ECL assay platform performs as follows:
1. Wash plate 3× with PBS-TWEEN.
2. Add coating solution to the well. (Coating solution contains capture antibody.)
3. Incubate for one hour with shaking at 705 rpm.
4. Wash plate 3× with PBS-TWEEN.
5. Add assay diluent.
6. Add calibrators.
7. Incubate for one hour with shaking at 705 rpm.
8. Wash plate 3× with PBS-TWEEN.
9. Add each detection antibody solution.
10. Incubate for one hour with shaking at 705 rpm.
11. Wash plate 3× with PBS-TWEEN.
12. Add read buffer to each well.
13. Read plate on imager.

### Example 3. Ultrasensitive Assays for the Detection of Brain-Derived Multi-Phosphorylated Tau (PTau181, pTau217 and pTau231) in Human Serum and Plasma

This Example demonstrates an ultrasensitive assay format with improved limits of detection and the capacity to interrogate multiple epitopes simultaneously. Assays were designed for human tau with specificity towards combinations of 1-3 phosphorylation sites: threonine 181 [pTau181], threonine 217 [pTau217], threonine 231 [pTau231], and/or a brain-specific pTau441 epitope. Limits of detection for brain-specific, or brain-associated, tau single-phosphorylation site assays were 13 fg/mL for pTau181, 26 fg/mL for pTau217, and 21 fg/mL for pTau231; these values represent a 5- to 150-fold improvement on currently available ultrasensitive kits for these assays. Novel assays with specificity towards a combination of two phosphorylation sites yielded limits of detection of 17 fg/mL for pTau181+pTau217, 37 fg/mL for pTau181+pTau231, and 43 fg/mL for pTau217+pTau231, while the assay with specificity towards all three phosphorylation sites yielded a limit of detection of 185 fg/mL.

These assays were used to determine tau concentrations in commercially-sourced normal (n=38) and AD-positive (n=38) serum and EDTA plasma samples. The pTau181, pTau217, and pTau181+pTau217 assays found detectable levels of tau in all samples, with significantly elevated levels found in AD-positive samples (P<0.001). Assays with selectivity towards pTau231 detected tau in up to 40% of samples. The subset of samples that contained detectable levels of pTau231 is the same subset of samples that contained the highest measured levels of pTau217 and pTau181. Additionally, we found a high correlation between samples with the pTau231 assay and the pTau217+pTau231 and pTau181+pTau231 assays, indicating that most pTau231 found in these samples is on multi-phosphorylated tau.

This new set of seven assays offers a capacity to monitor multi-phosphorylation of tau. When paired with the improvements in sensitivity, these novel assays provide a route to use non-invasive serum and plasma samples in AD research.

### Example 4. 2-Ab Immunoassay for Tau

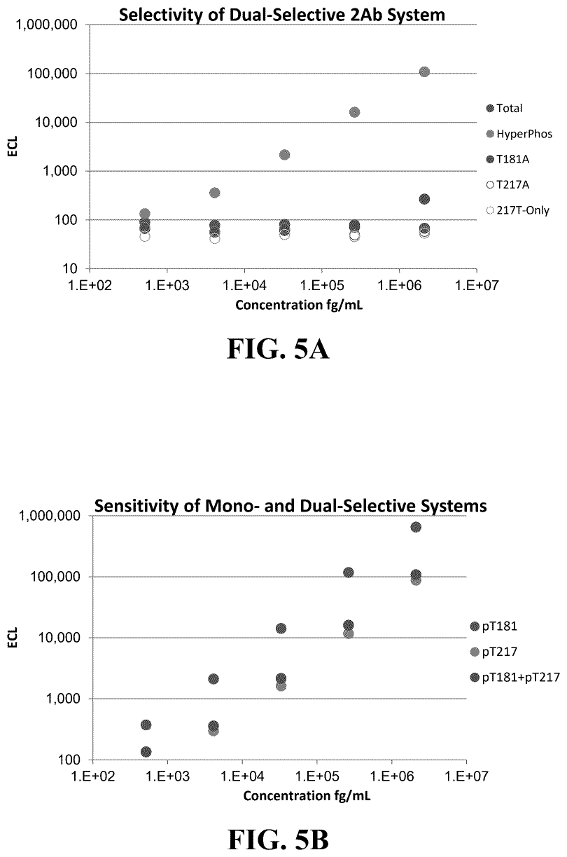

A 2-Ab immunoassay was developed to measure phosphorylated Tau (pTau) that is phosphorylated at both threonine181 (pTau181) and threonine 217 (pTau217) using a pTau181-specific antibody as capture reagent and a pTau217-specific antibody as detection reagent. This assay was compared to 2-Ab immunoassays specific for the individual phosphorylation site that paired capture reagents against pTau181 or pTau217 with a detection reagent that binds a non-phosphorylation dependent tau epitope. The dual-phosphorylation-specific assay showed high signal with a multi-phosphorylated Tau441 calibrator with high phosphorylation levels at both sites (labeled as “HyperPhos” in the graph shown in FIG. 5A). Excellent selectivity was demonstrated against tau forms that were not phosphorylated at both sites including an unphosphorylated calibrator (labeled as Total), a tau calibrator with all threonines replaced with alanines except at position 217 (labeled as 217T-Only), and two tau calibrators with the phosphorylation site at threonine 181 or 217 replaced with alanine (labeled as T181A and T217A) (FIG. 5A).

Signals for the dual-phosphorylation specific assay with the multi-phosphorylated calibrator (pT181+pT217) were indistinguishable from the signals for the assay for the pTau217 modification, but both provided less signals than the assay for the pTau218 modification (FIG. 5B), suggesting that the pTau217 modification may be present at lower levels in this material than the pTau181 modification.

### Example 5. Comparison of Sensitivity of 2-Ab and 3-Ab Immunoassays

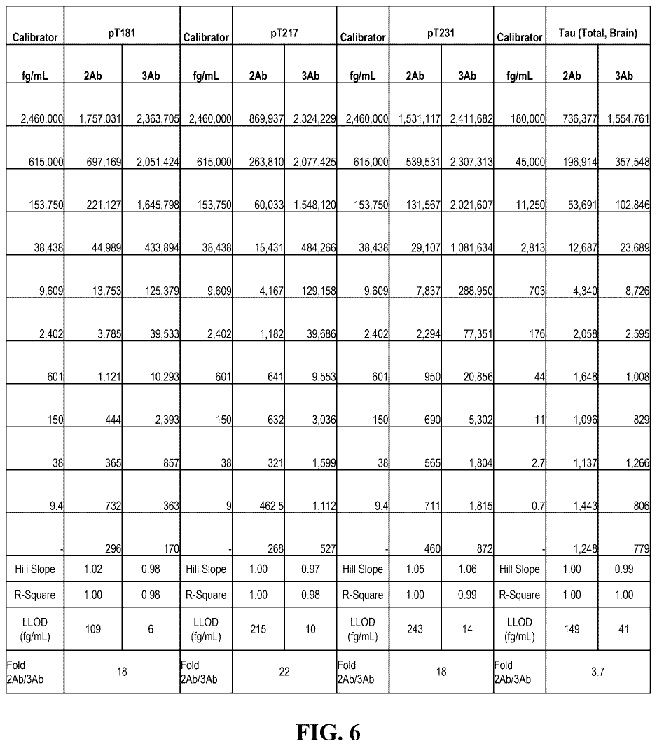

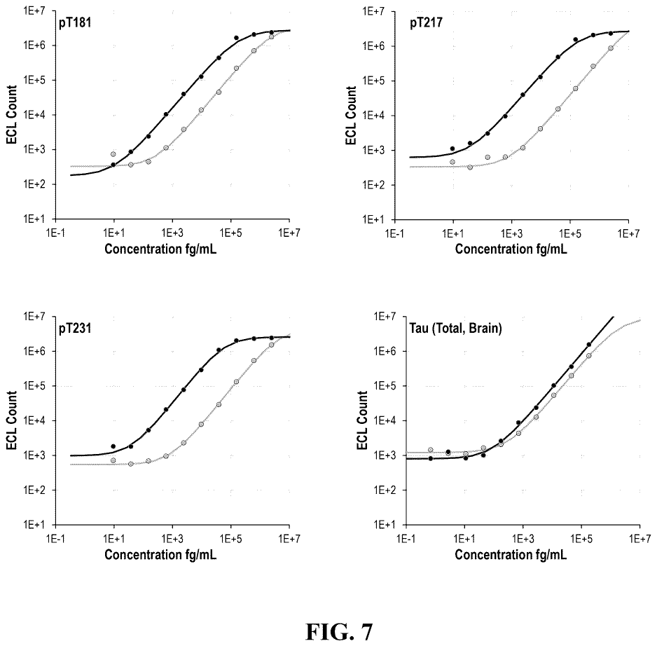

A summary of the improvement in assay sensitivity for the 3-Ab immunoassay over the 2-Ab immunoassay is provided in FIG. 6 and FIG. 7. FIG. 6 summarizes LLOD values for 3-Ab vs. 2-Ab immunoassays for pT181, pT217, pT231 and Tau (Total, Brain). FIG. 7 provides graphs of the results of 3-Ab (black line) vs. 2-Ab (grey line) immunoassays for pT181, pT217, pT231 and Tau (Total, Brain). Overall, a significant increase in sensitivity was observed in the 3-Ab immunoassay format over the 2-Ab immunoassay.

### Example 6. Detection of Phosphorylated Tau Using 3-Ab Immunoassays

The 3-Ab immunoassay can target three epitopes at once on a target analyte. Phosphorylation of Tau is indicative of neurological disease, with increased phosphorylated Tau levels indicating advancement in some diseases. Assays targeting 1 to 3 phosphorylated sites (pTau181, pTau217, pTau231, and two- or three-site combinations thereof) were developed and tested with 28 normal and 28 Alzheimer's Disease (AD)-positive serum samples, or 10 normal and 10 AD-positive plasma samples.

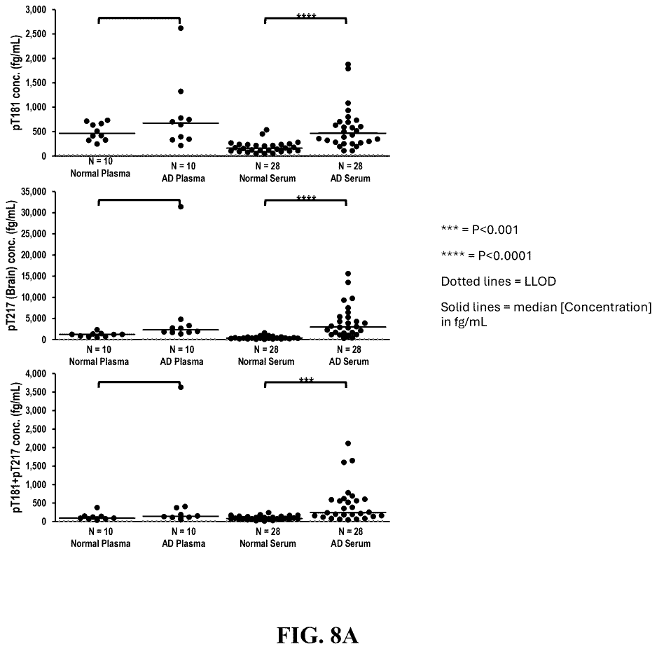

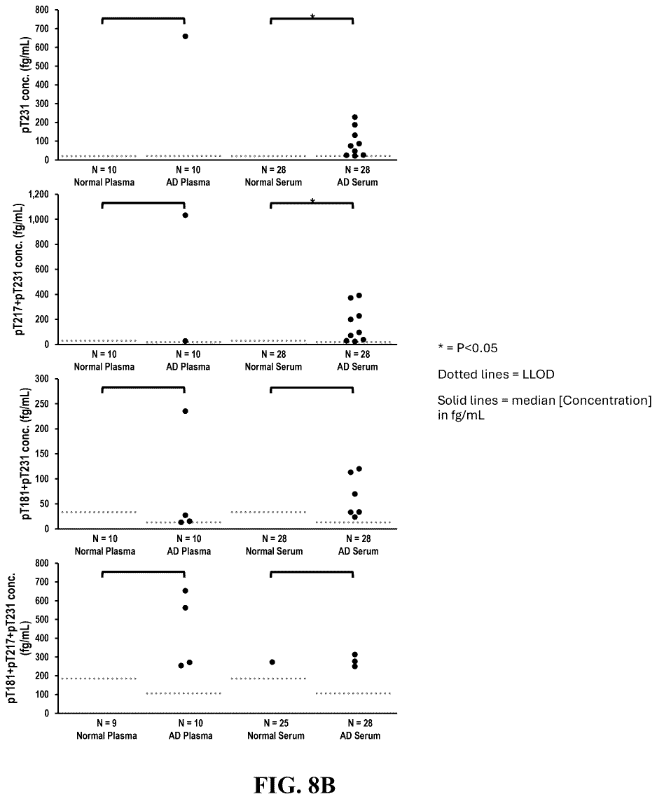

FIG. 8A shows comparisons for the pT181, pT217, and pT181+pT217 assays performed on normal and AD-positive plasma and serum samples. FIG. 8B shows comparisons for pT231-containing assays (pT231, pT181+pT231, pT217+pT231, and pT181+pT217+pT231) performed on normal and AD-positive plasma and serum samples. The serum assays showed significant differentiation for all assays except pT181+pT231 and pT181+pT217+pT231.

The results indicate low detection rates for pT231 despite a low LLOD (21 fg/mL). Phosphorylation at T231 appears to be predominantly paired with either pT181 or pT217. Further, pT181 is mostly paired with pT231, and p217 is mostly free (unpaired with other phosphorylation sites). Detectable levels of pT231 are only found if pT217 levels are also high.

The results further demonstrate that pT181 or pT217 alone is sufficient to differentiate normal from AD-positive samples in serum with P<0.0001 for each site. The pT217+pT231 and pT181+pT231 assays identified the samples with the most multi-phosphorylation, which may be useful in monitoring the progression of AD.

1. A method of detecting, quantifying, or both, phosphorylated tau (p-tau) in a biological sample, wherein the p-tau is phosphorylated at two or more sites, comprising:
(a) contacting the biological sample with: (i) a capture reagent that binds a phosphorylated tau site, or a non-phosphorylated tau site; (ii) a first detection reagent that binds a phosphorylated tau site, or a non-phosphorylated tau site; and
(iii) a second detection reagent that binds a phosphorylated tau site, or a non-phosphorylated tau site;
(b) forming a complex comprising the capture reagent, the p-tau, the first detection reagent and the second detection reagent; and
(c) detecting the complex, thereby detecting the p-tau; or
(d) measuring an amount of the complex, thereby quantifying an amount of the p-tau,
wherein at least two of the capture reagent, first detection reagent, and second detection reagent binds a phosphorylated tau site.

2. The method of claim 1, wherein at least one of the capture reagent, first detection reagent, or second detection reagent binds a brain-associated tau polypeptide.

3-8. (canceled)

9. The method of claim 1, wherein the capture reagent, the first detection reagent, and the second detection reagent comprise an antibody or antigen-binding fragment thereof, antigen, ligand, receptor, oligonucleotide, hapten, epitope, mimotope, or an aptamer.

10-12. (canceled)

13. The method of claim 1, wherein (i) the capture reagent binds pTau181, pTau205, pTau212, pTau217 or pTau231, and does not bind non-phosphorylated tau; or
(ii) either the first or the second detection reagent binds pTau181, pTau205, pTau212, pTau217, or pTau231, and does not bind non-phosphorylated tau; or
(iii) any combination of (i) and (ii).

14-22. (canceled)

23. The method of claim 1, wherein the capture reagent binds a surface,
thereby forming the complex on the surface comprising the capture reagent, the p-tau, the first detection reagent and the second detection reagent,
wherein the first detection reagent or second detection reagent comprises a detectable label.

24-29. (canceled)

30. The method of claim 1, wherein the biological sample comprises whole blood, blood serum plasma, cerebrospinal fluid (CSF), urine, saliva, or an extraction or purification therefrom, or dilution thereof.

31. A method of detecting a brain injury, brain injury severity, or a neurodegenerative disease in a subject, the method comprising obtaining measured levels of phosphorylated tau (p-tau) in a sample from the subject according to claim 1, wherein the sample is whole blood, cerebrospinal fluid (CSF), serum, plasma, or a combination thereof.

32. The method of claim 31, wherein the method detects
(i) brain injury or brain injury severity and the brain injury is concussive injury, subconcussive injury, acute concussive injury, impact head injury, acceleration or deceleration head trauma, closed-skull neurotrauma, traumatic brain injury, stroke, seizure, status epilepticus, chronic traumatic encephalopathy (CTE), or a combination thereof; or
(ii) a neurodegenerative disease and the neurodegenerative disease is Alzheimer's disease, amyotrophic lateral sclerosis, Friedreich ataxia, Huntington's disease, Lewy body disease, Parkinson's disease, spinal muscular atrophy, Creutzfeldt-Jakob disease, Neuronal Synuclein Disease (NSD), motor neuron disease, or any combination thereof.

33. (canceled)

34. (canceled)

35. A method of determining eligibility of a subject to participate in a clinical trial of a therapeutic drug for preventing or delaying Alzheimer's disease, the method comprising:
(a) obtaining a measurement of phosphorylated tau levels of a sample from the subject according to claim 1; and
(b) determining the eligibility of the subject for the clinical trial based on the measurement of phosphorylated tau levels;
wherein the subject has been diagnosed with dementia or mild cognitive impairment, or wherein the subject has subjective cognitive complaints, or wherein the subject does not have any cognitive impairment; or
a method of conducting a clinical trial of a therapeutic drug or intervention for Alzheimer's disease, the method comprising:
(a) obtaining a measurement of phosphorylated tau levels of a sample from a subject according to claim 1:
(b) determining eligibility of the subject for the clinical trial based on the measurement of phosphorylated tau levels; and
(c) administering the therapeutic drug to the subject: or
a method of distinguishing a subject afflicted with Alzheimer's disease from an individual afflicted with non-Alzheimer's dementia, the method comprising:
(a) obtaining a measurement of phosphorylated tau levels of a sample from the subject according to claim 1; and
(b) identifying, based on the measurement of phosphorylated tau levels, the subject as (i) afflicted with Alzheimer's disease or (ii) afflicted with non-Alzheimer's dementia; or
a method of treating Alzheimer's disease in a subject in need thereof, the method comprising:
(a) obtaining a measurement of phosphorylated tau levels of a sample from the subject according to claim 1, wherein the measurement is obtained prior to administration of a treatment for Alzheimer's disease,
(b) determining, based on the measurement of phosphorylated tau levels, that the subject is afflicted with Alzheimer's disease, and
(c) administering a treatment regimen for Alzheimer's disease to the subject; or
a method of monitoring response to treatment for Alzheimer's disease in a subject, the method comprising:
(a) obtaining a first measurement of phosphorylated tau levels of a sample from the subject according to claim 1, wherein the first measurement is obtained prior to administration of a treatment regimen for Alzheimer's disease,
(b) obtaining a second measurement of phosphorylated tau levels of a sample from the subject at one or more time points after administration of the treatment regimen for Alzheimer's disease has been initiated,
(c) determining, based on the first and second measurements of phosphorylated tau levels, that the subject is responding positively to the Alzheimer's treatment regimen, and
(d) continuing to administer the treatment regimen for Alzheimer's disease to the subject.

36-39. (canceled)

40. A kit for detecting phosphorylated tau (p-tau) in a biological sample, wherein the p-tau is phosphorylated at two or more sites, comprising, in one or more vials, containers, or compartments, three reagents:
(a) a capture reagent that binds a phosphorylated tau site, or a non-phosphorylated tau site;
(b) a first detection reagent that binds a phosphorylated tau site, or a non-phosphorylated tau site; and
(c) a second detection reagent that binds a phosphorylated tau site, or a non-phosphorylated tau site, wherein at least two of the three reagents bind a phosphorylated tau site.

41. The kit of claim 40, wherein the p-tau comprises: T175 (pTau175), T181 (pTau181), T212 (pTau212), T205 (pTau205), S214 (pTau214), T217 (pTau217), cis or trans T231 (pTau231), S293 (pTau293), 5396 (pTau396), L243 (Tau243) or any combination thereof, with reference to SEQ ID NO: 1.

42. The kit of claim 41, comprising:
(a) a capture reagent that binds pTau217;
(b) a first detection reagent that binds pTau231; and
(c) a second detection reagent that binds pTau181, Tau243, or a brain-associated tau polypeptide comprising the amino acid sequence of SEQ ID NO: 2 (HVTQARMV) or SEQ ID NO: 3 (AGHVTQARMVSK); or
(a) a capture reagent that binds pTau217;
(b) a first detection reagent that binds pTau231; and
(c) a second detection reagent that binds a brain-associated tau polypeptide comprising the amino acid sequence of SEQ ID NO: 2 (HVTQARMV) or SEQ ID NO: 3 (AGHVTQARMVSK), or
(a) a capture reagent that binds pTau217;
(b) a first detection reagent that binds pTau181; and
(d) a second detection reagent that binds pTau231.

43. (canceled)

44. (canceled)

45. The kit of claim 40, further comprising a surface, a detectable label, an anchoring reagent, a labeled probe, an immobilizing reagent, a template oligonucleotide, a polymerase, a ligase, a buffer, a blocking agent, a co-reactant, a diluent, a stabilizing agent, a calibration agent, an assay consumable, an electrode or any combination thereof.

46. The kit of claim 40, wherein each capture reagent and detection reagent comprises an antibody or antigen-binding fragment thereof.

47-51. (canceled)

52. A system comprising: one or more data processors; and a non-transitory computer readable storage medium containing instructions which, when executed on the one or more data processors, cause the one or more data processors to perform part or all of the method of claim 1.

53. A computer-program product tangibly embodied in a non-transitory machine-readable storage medium, including instructions configured to cause one or more data processors to perform part or all of the method of claim 1.

54. A method of detecting, quantifying, or both, tau in a biological sample, wherein the tau comprises a plurality of detectable epitopes, the method comprising:
(a) contacting the biological sample with a plurality of binding reagents comprising a first binding reagent, a second binding reagent, and a third binding reagent, wherein the first binding reagent binds to a first epitope, the second binding reagent binds to a second epitope, and the third binding reagent binds to a third epitope;
(b) forming a complex comprising the plurality of binding reagents and the tau; and
(c) detecting the complex, thereby detecting the tau; or
(d) measuring an amount of the complex, thereby quantifying an amount of the tau.

55. The method of claim 54, wherein (i) the first epitope comprises a first site capable of being phosphorylated: (ii) the second epitope comprises a second site capable of being phosphorylated; (iii) the third epitope comprises a third site capable of being phosphorylated: or (iv) any combination of (i) to (iii).

56. (canceled)

57. (canceled)

58. The method of claim 55, wherein (i) the first binding reagent binds to the phosphorylated state of the first site or binds to the non-phosphorylated state of the first site: (ii) the second binding reagent binds to the phosphorylated state of the second site or binds to the non-phosphorylated state of the second site: (iii) the third binding reagent binds to the phosphorylated state of the third site or binds to the non-phosphorylated state of the third site; or (iv) any combination of (i) to (iii).

59-66. (canceled)

67. The method of claim 55, further comprising a fourth binding reagent.

68. The method of claim 67, wherein the first binding reagent binds to the phosphorylated state of the first site and/or the fourth binding reagent binds to the non-phosphorylated state of the first site.

69-83. (canceled)

84. The method of claim 54, wherein the first binding reagent is a first capture reagent.

85. The method of claim 84, further comprising a second capture reagent and/or an anchoring reagent.

86-98. (canceled)

99. The method of claim 55, wherein the biological sample comprises:
a first population of tau comprising the first epitope comprising the phosphorylated state of the first site; and
a second population comprising the first epitope comprising the non-phosphorylated state of the first site.

100. (canceled)

101. A kit for detecting, quantifying, or both, tau in a biological sample, where the tau comprises at least one detectable epitope comprising a first site, wherein the kit comprises:
(a) a first binding reagent that binds to the phosphorylated state of the first site; and
(b) a second binding reagent that binds to the non-phosphorylated state of the first site.

102. The kit of claim 101, wherein the first binding reagent and/or the second binding reagent is an antibody or antigen-binding fragment thereof.

103-106. (canceled)


SEQUENCE LISTING (9 sequences)

SEQ ID NO: 1 - protein, 441 aa, Homo sapiens
  REGION 1..441: note = amino acid sequence of human CNS tau protein (2N4R)
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 8 aa, Homo sapiens
  REGION 1..8: note = amino acid sequence of human brain-associated tau polypeptide
HVTQARMV

SEQ ID NO: 3 - protein, 12 aa, Homo sapiens
  REGION 1..12: note = amino acid sequence of human brain-associated tau polypeptide (for e.g., for immunizations)
AGHVTQARMVSK

SEQ ID NO: 4 - protein, 758 aa, Homo sapiens
  REGION 1..758: note = amino acid sequence of human PNS tau (Big tau) protein
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 5 - protein, 352 aa, Homo sapiens
  REGION 1..352: note = amino acid sequence of human CNS tau protein (0N3R)
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 6 - protein, 381 aa, Homo sapiens
  REGION 1..381: note = amino acid sequence of human CNS tau protein (1N3R)
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 7 - protein, 410 aa, Homo sapiens
  REGION 1..410: note = amino acid sequence of human CNS tau protein (2N3R)
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 8 - protein, 383 aa, Homo sapiens
  REGION 1..383: note = amino acid sequence of human CNS tau protein (0N4R)
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 9 - protein, 412 aa, Homo sapiens
  REGION 1..412: note = amino acid sequence of human CNS tau protein (1N4R)
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL